(19) United States
(12) Patent Application Publication
(10) Pub. No.: US 2024/0409570 A1
(43) Pub. Date: Dec. 12, 2024

(54) SYNTHETIC COMPOUND, KIT COMPRISING THE SAME, AND USES THEREOF

(71) Applicants: Academia Sinica, Taipei City (TW); Chang Gung Memorial Hospital, Linkou, Taoyuan City (TW); Taipei Veterans General Hospital, Taipei City (TW)

(72) Inventors: Yun-Ru CHEN, Taipei City (TW); Chung-Yi WU, Taipei City (TW); Hwai-I YANG, Taipei City (TW); Chiung-Mei CHEN, Taoyuan City (TW); Pei-Ning WANG, Taipei City (TW)

(21) Appl. No.: 18/690,712

(22) PCT Filed: Sep. 8, 2022
(86) PCT No.: PCT/US2022/042938

§ 371 Date: Mar. 8, 2024

Related U.S. Application Data
(60) Provisional application No. 63/243,099, filed on Sep. 11, 2021.

(51) Int. Cl. C07H 15/04; A61K 31/7028; G01N 33/68

(52) U.S. Cl. CPC C07H 15/04; A61K 31/7028; G01N 33/6896; G01N 2400/00; G01N 2800/2821; G01N 2800/2835

(57) ABSTRACT

Disclosed herein is a compound and its use for the prognosis or diagnosis of neurodegenerative diseases. The compound has the structure of formula (I),

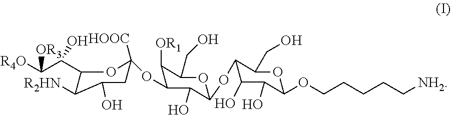

According to embodiments of the present disclosure, the neurodegenerative disease may be an Alzheimer's disease (AD), Parkinson disease (PD), Huntington's disease (HD), frontotemporal dementia (FTD), Friedreich's ataxia, age-related macular degeneration, or Creutzfeldt-Jakob disease.

### CROSS-REFERENCE TO RELATED APPLICATIONS

This application relates to and claims the benefit of U.S. Provisional Application No. 63/243,099, filed Sep. 11, 2021; the content of the application is incorporated herein by reference in its entirety.

### BACKGROUND OF THE INVENTION

### 1. Field of the Invention

The present disclosure in general relates to the prognosis and/or diagnosis of diseases. More specifically, the present disclosure relates to the prognosis and/or diagnosis of neurodegenerative disease by use of a synthetic ganglioside.

### 2. Description of Related Art

Neurodegeneration is attributed from progressive loss of structure and function of neurons including synaptic dysfunction and neuronal apoptosis. Neurodegenerative process in different brain regions results in different neurodegenerative diseases, such as the most prevalent dementia in the elderly, Alzheimer's disease (AD), that affected several tens of millions people, and the motor neuron degeneration, Parkinson's (PD) and Huntington's diseases (HD). Dementia describes significant loss of certain mental functions such as memory, attention, and abstract thinking. The most prevalent dementia is AD. In the case of AD, there are five most widely studied biomarkers of AD pathology from cerebrospinal fluid (CSF) and brain imaging. They are decreased Aβ42 in CSF, increased phosphorylated tau and total tau in CSF, decreased fluorodeoxyglucose uptake on PET, PET amyloid imaging, and structural MRI measures of cerebral atrophy. However, no blood-based biomarker including Aβ level so far can be identified for helping diagnosis of AD. Nonetheless, analysis of plasma instead of CSF is highly desirable because of its accessibility and less invasive sampling procedure.

HD is characterized by cognitive decline, movement disorder, and behavioral abnormalities. HD is autosomal-dominant inherited and marked by an abnormal CAG tri-nucleotide repeat expansion in the gene encoding Huntingtin, a ubiquitously expressed protein. In HD patients, CAG repeat number is more than 35, whereas, the normal CAG repeats is under 35. Studies showed that the longer the CAG repeats, the earlier the disease onset. However, genetic testing for HD can only detect the individuals at risk, but cannot predict the disease onset. Early HD clinical biomarker is important especially for the pre-manifest stages of HD (pre-HD), because identifying pre-HD and early stage HD allows therapeutic intervention for individuals before development of disease symptoms.

Gangliosides are sialic acid-containing glycosphingolipids (GSLs) that are expressed in the outer leaflet of plasma membrane of all vertebrate cells and are most enriched in the nervous system. Gangliosides are involved in a variety of functions, including serving as antigens, receptors for bacterial toxins, mediators of cell adhesion, and mediators and modulators of signal transduction. The expression in the nervous system is cell specific and developmentally regulated, and their quantities and species undergo dramatic changes during the differentiation of the cell. Gangliosides are known to play an important role in neuronal development and regeneration, whereas anti-ganglioside antibodies have been shown to impair these processes. Autoimmunity occurs when the immune tolerance mechanisms fail and self-antigens are being recognized by autoantibodies or cellular components. Alteration of brain ganglioside patterns has been observed in the pathology of many neurodegenerative diseases, including AD, PD and HD. Anti-ganglioside antibodies have been described in plasma of patients with peripheral neuropathy and a number of immune-mediated neurological diseases.

As aforementioned, early diagnosis of neurodegenerative diseases is critical for possible pharmaceutical treatments and improving patients' healthier lives. Discovery of sensitive and specific biomarkers for neurodegenerative diseases will be tremendously useful to facilitate the clinical diagnosis. An ideal biomarker should indicate specific features of disease-related pathological changes and be non-invasive, cost-effective, and sensitive to the detection method.

In view of the above, there exists in this art a need of a novel biomarker for making a prognosis and/or early diagnosis of neurodegenerative diseases so as to improve the life quality and life span of patients.

### SUMMARY

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

In general, the present disclosure relates to the five synthetic ganglio-oligosaccharides, each of which may serve as a biomarker for the prognosis and/or diagnosis of neurodegenerative diseases. Accordingly, the present disclosure also provides a method of making a prognosis and/or diagnosis of neurodegenerative diseases by use of the synthetic ganglio-oligosaccharides.

The first aspect of the present disclosure aims at providing a compound having the structure of formula (I),

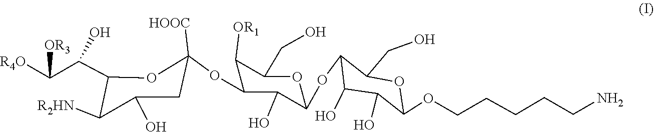

wherein,
R₁ is H, or optionally substituted

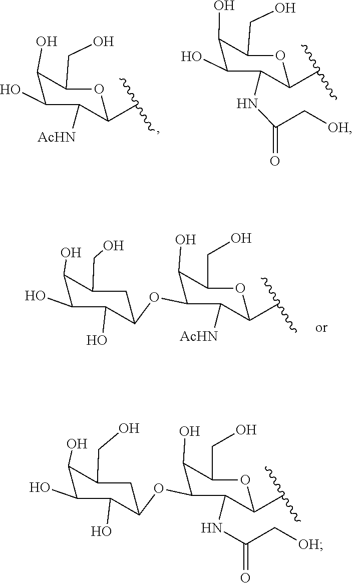 [SMILES: *C1OC(CO)C(O)C(O)C1NC(C)=O ; *C1OC(CO)C(O)C(O)C1NC(=O)CO ; *C1OC(CO)C(O)C(OC2CC(CO)C(O)C(O)C2O)C1NC(C)=O ; *C1OC(CO)C(O)C(OC2CC(CO)C(O)C(O)C2O)C1NC(=O)CO]

R₂ is optionally substituted acetyl or

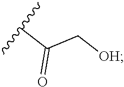 [SMILES: *C(=O)CO]

and
R₃ and R₄ are independently H, or optionally substituted

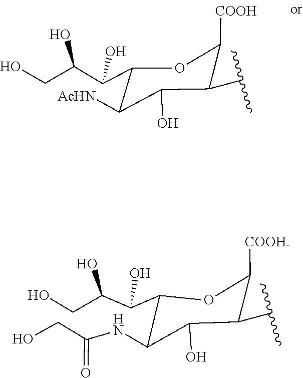 [SMILES: *C1C(C(=O)O)OC([C@H](O)[C@H](O)CO)C(NC(C)=O)C1O ; *C1C(C(=O)O)OC([C@H](O)[C@H](O)CO)C(NC(=O)CO)C1O]

According to some working embodiments, the compound of formula (I) is any of the followings,

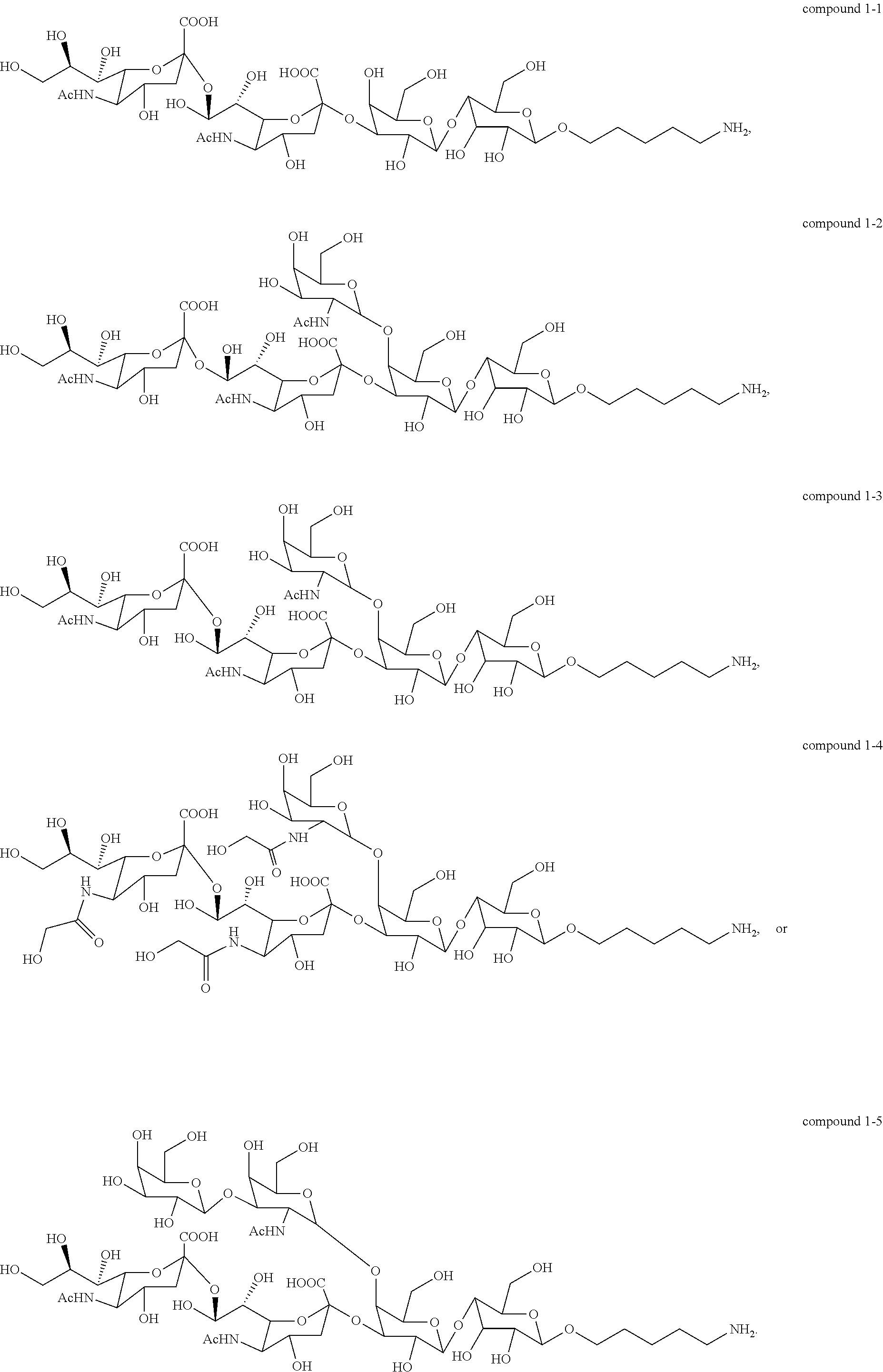

The second aspect of the present disclosure pertains to a pharmaceutical kit for making a prognosis and/or diagnosis of neurodegenerative diseases. The present pharmaceutical kit comprises two compounds, one of which has the structure of formula (I), and the other of which is selected from the group consisting of,

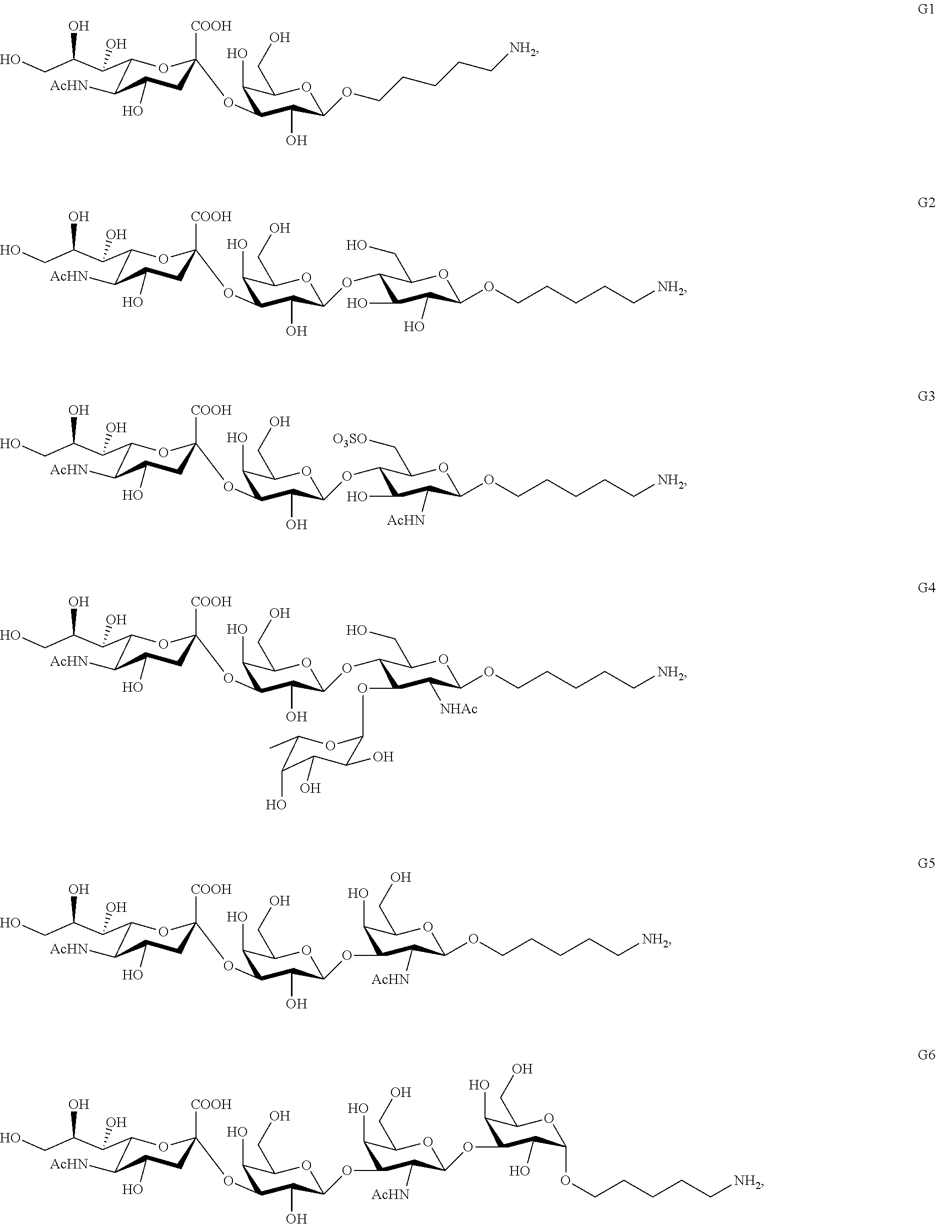

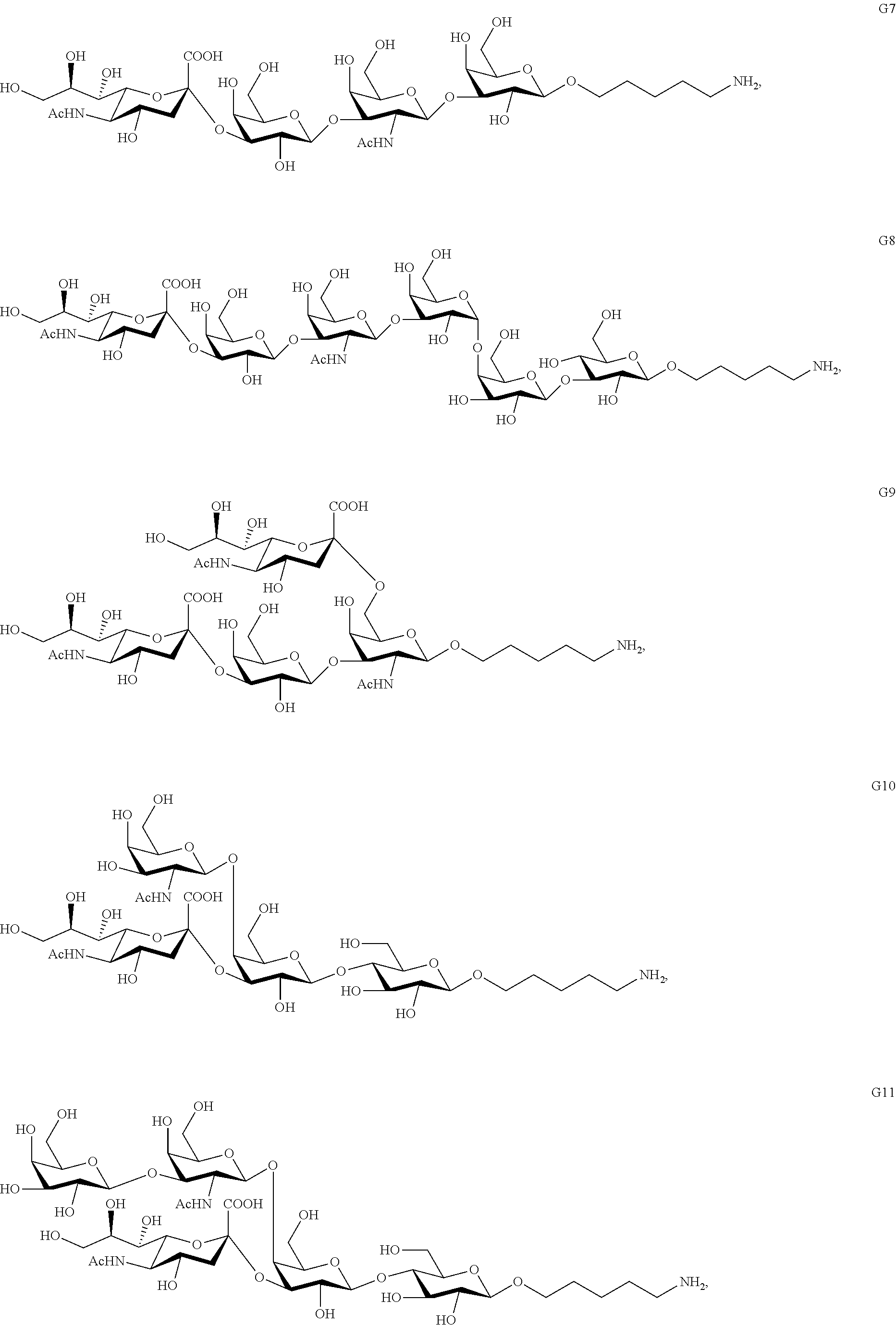

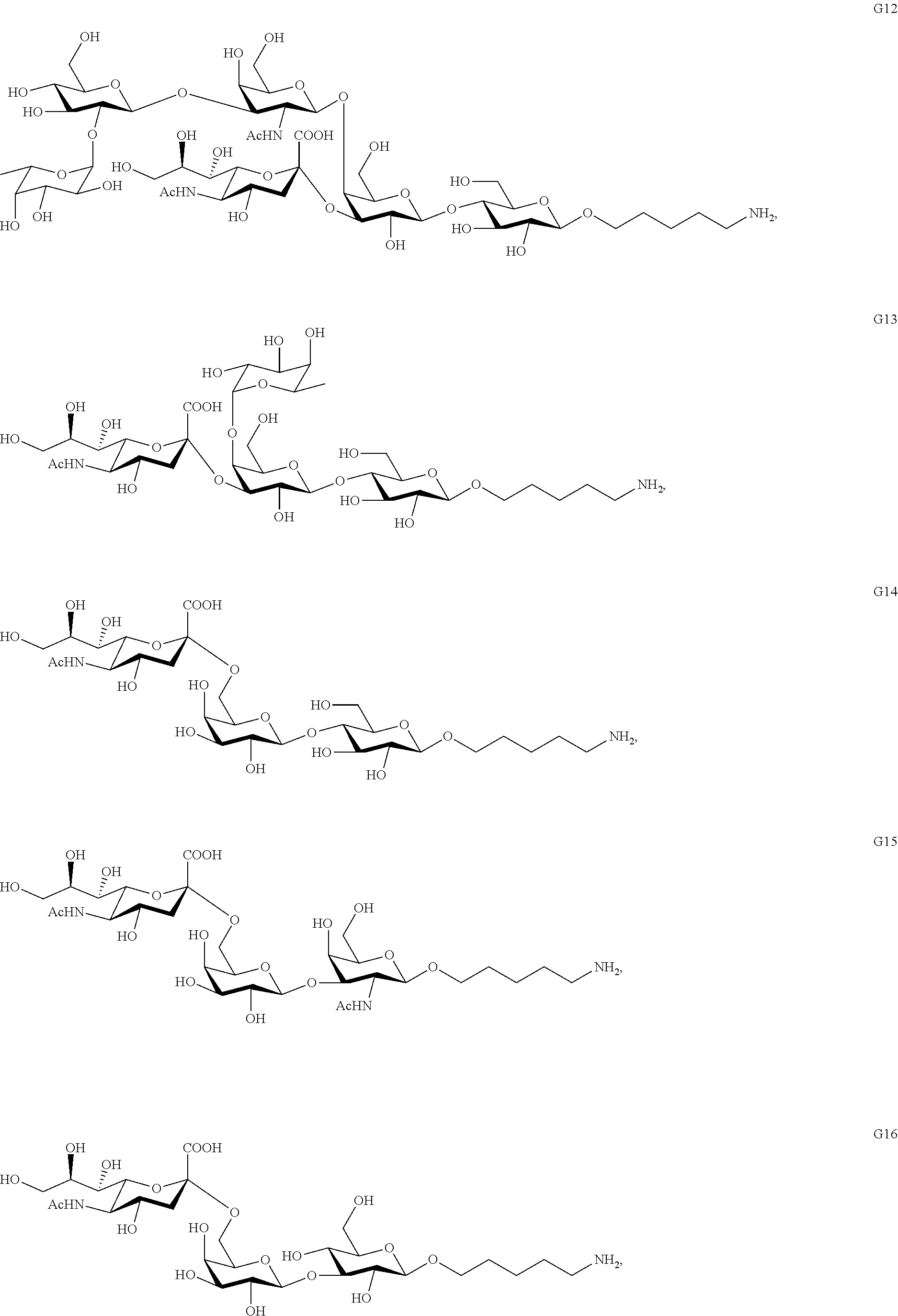

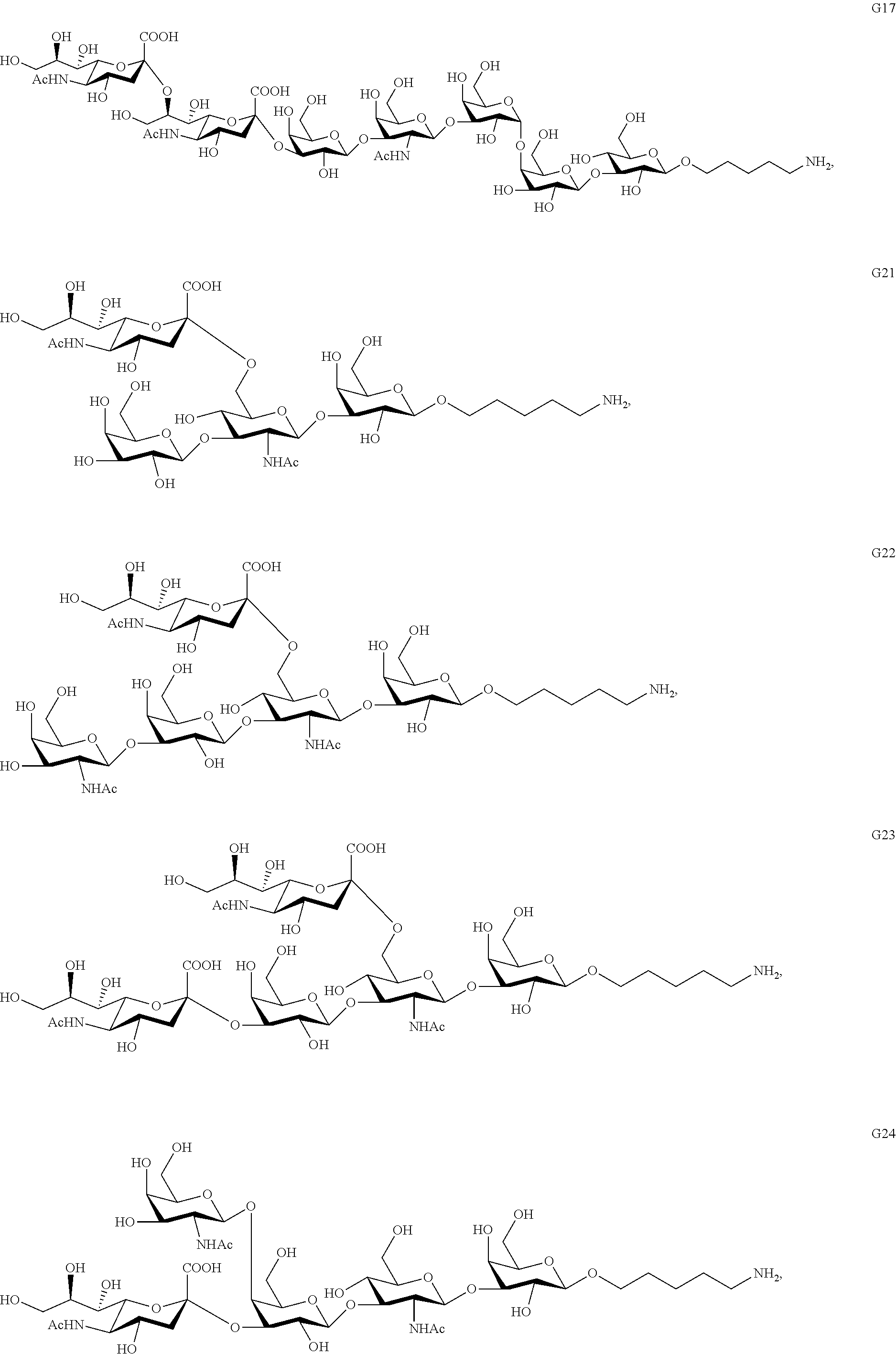

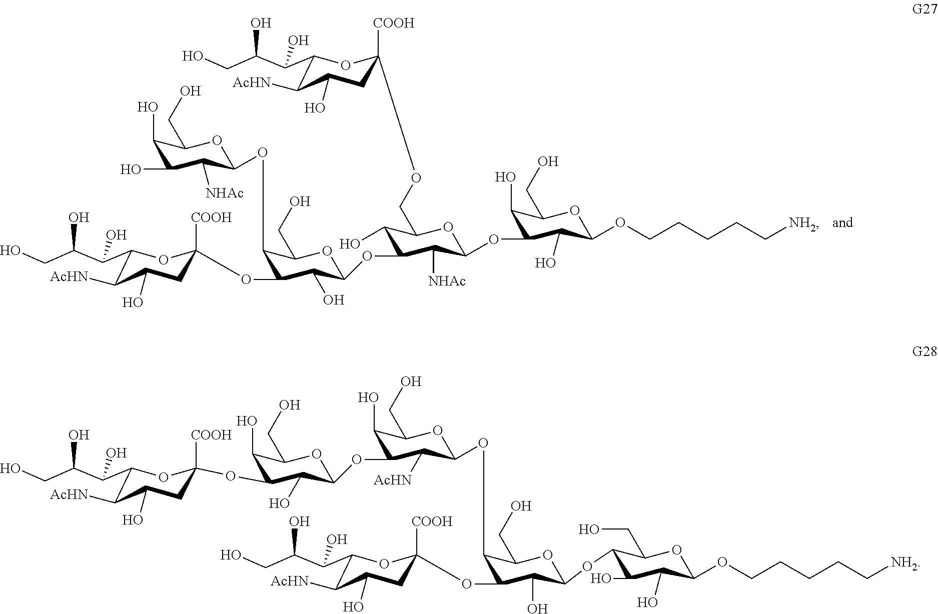

Also disclosed herein is a method of making a prognosis or diagnosis of a neurodegenerative disease via a biological sample obtained from a subject. The method comprises,
(a) mixing the biological sample and the compound of formula (I) to form a first immunocomplex;
(b) reacting an anti-IgM antibody with the first immunocomplex of step (a) to give a second immunocomplex, wherein the anti-IgM antibody is conjugated with a reporter molecule;
(c) determining the signal level of the reporter of the step (b); and
(d) making the prognosis or diagnosis of the neurodegenerative disease based on the determination made in the step (c), wherein when the signal level is higher than that of a reference sample, then then subject has or is at risk of having the neurodegenerative disease.

Another aspect of the present disclosure is directed to a method of treating a neurodegenerative disease in a subject, comprising,
(a) obtaining a biological sample from the subject;
(b) mixing the biological sample of step (a) and the compound of formula (I) to form a first immunocomplex;
(c) reacting an anti-IgM antibody with the first immunocomplex of step (b) to give a second immunocomplex, wherein the anti-IgM antibody is conjugated with a reporter molecule;
(d) determining the signal level of the reporter of the step (c); and
(e) administering to the subject an effective amount of an anti-neurodegenerative treatment based on the determination made in the step (d), wherein the signal level in the biological sample of the subject is higher than that of a reference sample.

The biological sample may be a whole blood sample, a serum sample, or a plasma sample.

According to some working examples of the present disclosure, the reference sample is derived from a healthy subject.

Depending on desired purposes, the reporter molecule conjugated with the anti-IgM antibody may be a tag molecule, a radioactive molecule, a fluorescent molecule, a phosphorescent molecule, a chemiluminescent molecule or an enzyme.

The neurodegenerative disease estimated and determined by the present compound, pharmaceutical kit and/or method may be any of Alzheimer's disease (AD), Parkinson disease (PD), Huntington's disease (HD), frontotemporal dementia (FTD), Friedreich's ataxia, age-related macular degeneration, or Creutzfeldt-Jakob disease.

The subject is a mammal; preferably, a human.

Many of the attendant features and advantages of the present disclosure will become better understood with reference to the following detailed description considered in connection with the accompanying drawings.

### BRIEF DESCRIPTION OF THE DRAWINGS

The present description will be better understood from the following detailed description read in light of the accompanying drawing, where:

FIG. 1 depicts the total IgM level in plasma of NC, pre-HD cases, and HD patients according to Example 1.1 of the present disclosure. The total IgM level were quantified by slot blotting after loading equal amount of plasma on the nitrocellulose membrane. Each dot indicated a single case. The statistical differences among NC, pre-HD cases, and HD patients were analyzed by one-way ANOVA and Tukey's Post Hoc Test.

### DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

### 1. Definitions

For convenience, certain terms employed in the context of the present disclosure are collected here. Unless defined otherwise, all technical and scientific terms used herein have the same meaning as commonly understood by one of the ordinary skill in the art to which this invention belongs.

As used herein, when the term “optionally substituted” is preceded by a selection of compounds (e.g., mono-or di-saccharides) means each compound in that selection is substituted or unsubstituted. For example, the term “optionally substituted

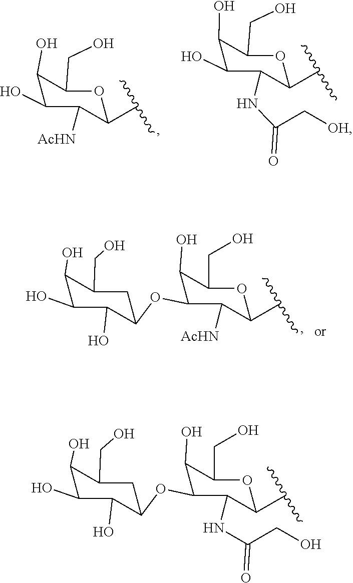 [SMILES: *C1OC(CO)C(O)C(O)C1NC(C)=O ; *C1OC(CO)C(O)C(O)C1NC(=O)CO ; *C1OC(CO)C(O)C(OC2CC(CO)C(O)C(O)C2O)C1NC(C)=O ; *C1OC(CO)C(O)C(OC2CC(CO)C(O)C(O)C2O)C1NC(=O)CO]

is same as “optionally substituted

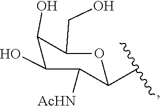 [SMILES: *C1OC(CO)C(O)C(O)C1NC(C)=O]

optionally substituted

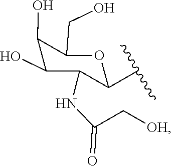 [SMILES: *C1OC(CO)C(O)C(O)C1NC(=O)CO]

optionally substituted

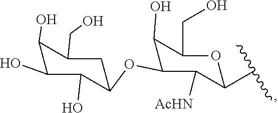 [SMILES: *C1OC(CO)C(O)C(OC2CC(CO)C(O)C(O)C2O)C1NC(C)=O]

or optionally substituted

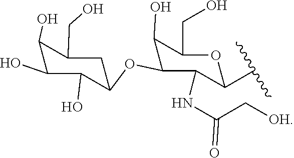 [SMILES: *C1OC(CO)C(O)C(OC2CC(CO)C(O)C(O)C2O)C1NC(=O)CO]

The term “substituted” is contemplated to include substitutions with all permissible substituents of organic compounds, any of the substituents described herein that results in the formation of a stable compound.

It should also be noted that names of compounds having one or more chiral centers that do not specify the stereochemistry of those centers encompass pure stereoisomers and mixtures thereof. Moreover, any atom shown in a drawing with unsatisfied valences is assumed to be attached to enough hydrogen atoms to satisfy the valences.

As used herein, the term “prognosis” refers to a prediction of the outcome of a condition, for example, a good or poor outcome (e.g., likelihood of developing a neurodegenerative disease). As could be appreciated, “prognosis” does not refer to the ability to predict the course or outcome of a condition with 100% accuracy. Instead, persons having ordinary skills in the art would understand that the term “prognosis” refers to an increased probability that a certain course or outcome will occur; that is, that a course or outcome is more likely to occur in a subject exhibiting a given condition (e.g., having higher expression level of anti-IgM antibodies against the present compound), when compared with those subjects not exhibiting the condition (e.g., having lower expression level of anti-IgM antibodies against the present compound). A favorable prognosis includes a prediction of low likelihood of developing a neurodegenerative disease (including AD and HD), while an unfavorable prognosis includes a prediction of high likelihood of developing the neurodegenerative disease.

The term “diagnosis” as used herein refers to methods by which the skilled artisan can estimate and/or determine the probability (“a likelihood”) of whether or not a patient is suffering from a given disease or condition. In the case of the present invention, “diagnosis” includes using the results of the expression level of the present compound, optionally together with other clinical characteristics, to arrive at a diagnosis (that is, the occurrence or nonoccurrence) of a neurodegenerative disease for the subject from which a sample was obtained and assayed. That such a diagnosis is “determined” is not meant to imply that the diagnosis is 100% accurate. Many biomarkers are indicative of multiple conditions. The skilled clinician does not use biomarker results in an informational vacuum, but rather test results are used together with other clinical indicia to arrive at a diagnosis. Thus, a measured biomarker level on one side of a predetermined diagnostic threshold indicates a greater likelihood of the occurrence of disease in the subject relative to a measured level on the other side of the predetermined diagnostic threshold.

The term “subject” refers to a mammal including the human species that may be estimated and determined by the compound, kit and/or method of the present disclosure. The term “subject” is intended to refer to both the male and female gender unless one gender is specifically indicated.

The term “healthy subject” refers to a subject that does not have a disease (e.g., neurodegenerative disease). For example, a healthy subject has not been diagnosed as having a disease and is not presenting with two or more (e.g., two, three, four or five) symptoms associated with the disease.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

The singular forms “a,” “and,” and “the” are used herein to include plural referents unless the context clearly dictates otherwise.

### 2. Detail Description Of Preferred Embodiments

The present disclosure is based, at least in part, on the discovery that the expression levels of some anti-ganglioside antibodies in a subject are correlated with the occurrence of neurodegenerative diseases (for example, AD, PD, HD, FTD, Friedreich's ataxia, age-related macular degeneration, and Creutzfeldt-Jakob disease), in which compared with the subject with low expression levels, the subject having high expression levels are more prone to developing neurodegenerative diseases. Accordingly, the present disclosure aims at providing several target compounds for detecting the anti-ganglioside antibodies in a subject thereby determining whether the subject is at risk of developing neurodegenerative diseases. Also disclosed are methods of evaluating the occurrence or the likelihood of occurrence of neurodegenerative diseases in a subject by use of the target compounds.

The first aspect of the present disclosure is thus directed to a compound having the structure of formula (I),

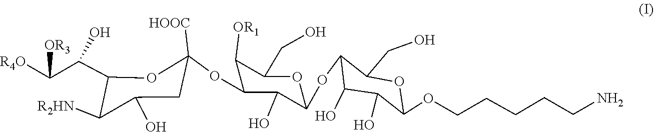

According to embodiments of the present disclosure,
R₁ is H, or optionally substituted

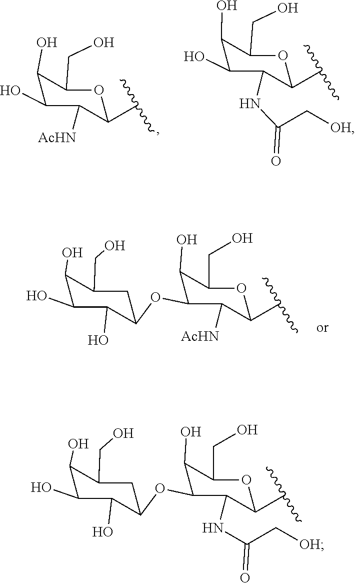 [SMILES: *C1OC(CO)C(O)C(O)C1NC(C)=O ; *C1OC(CO)C(O)C(O)C1NC(=O)CO ; *C1OC(CO)C(O)C(OC2CC(CO)C(O)C(O)C2O)C1NC(C)=O ; *C1OC(CO)C(O)C(OC2CC(CO)C(O)C(O)C2O)C1NC(=O)CO]

R₂ is optionally substituted acetyl or

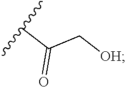 [SMILES: *C(=O)CO]

and
R₃ and R₄ are independently H, or optionally substituted

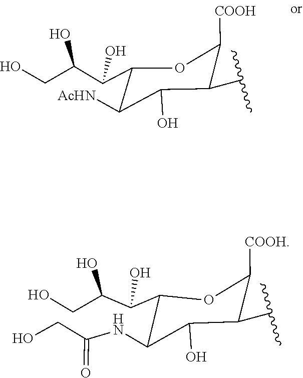 [SMILES: *C1C(C(=O)O)OC([C@H](O)[C@H](O)CO)C(NC(C)=O)C1O ; *C1C(C(=O)O)OC([C@H](O)[C@H](O)CO)C(NC(=O)CO)C1O]

According to some working examples of the present disclosure, the compound of formula (I) may be any of the followings:

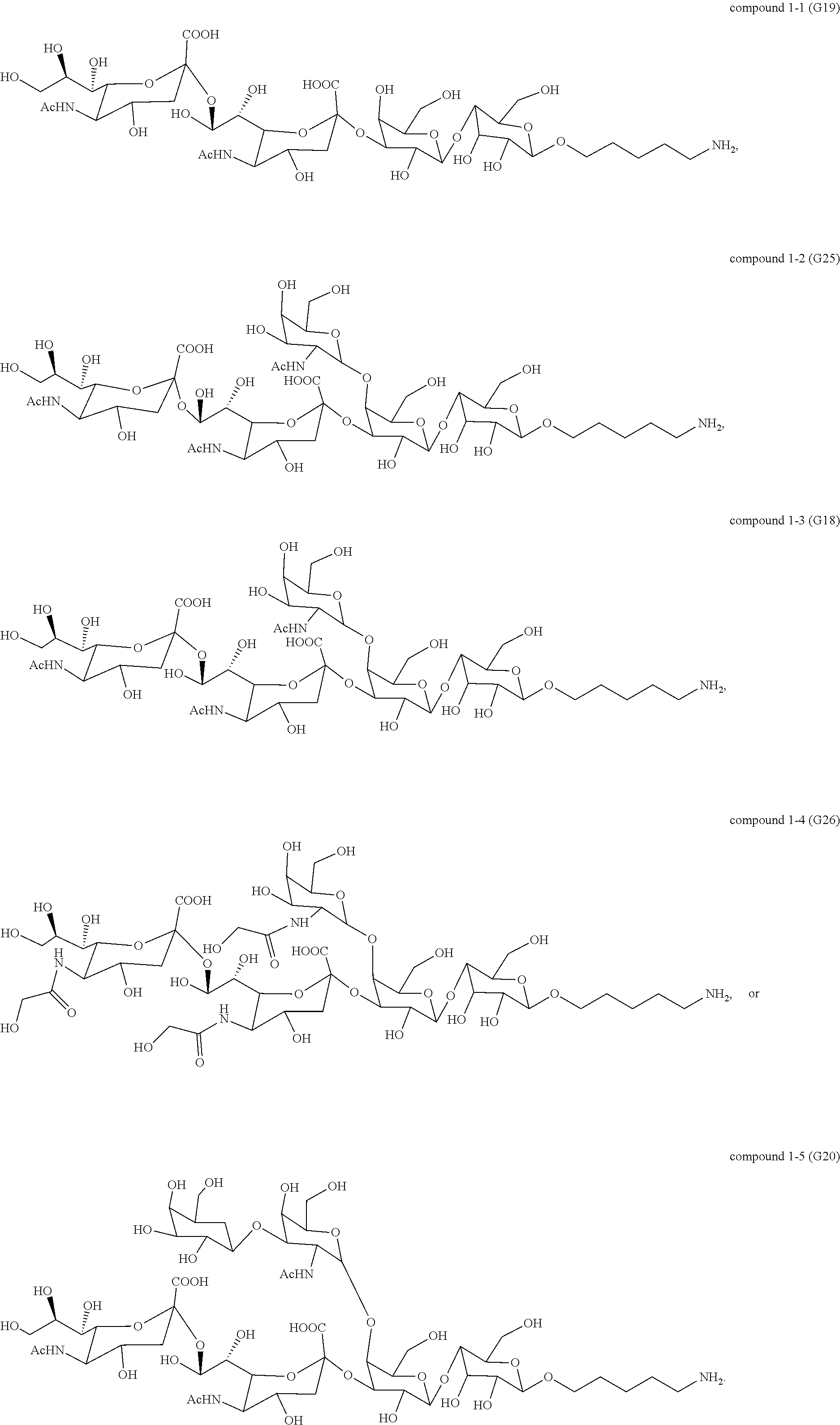

According to certain embodiments of the present disclosure, the compound of formula (I) is useful in identifying the subject having mild cognitive impairment (MCI), in which the expression level of anti-IgM antibody specific to the compound of formula (I) is higher in the subject having MCI than that in the healthy subject or AD patient.

According to some embodiments of the present disclosure, the compound of formula (I) serves as a biomarker for identifying the subject with pre-manifest stages of HD (pre-HD; i.e., a subject having no clinical symptom of HD), in which compared with the healthy subject and HD patient, the pre-HD subject has higher expression level of anti-IgM antibody specific to the compound formula (I).

The second aspect of the present disclosure is directed to a pharmaceutical kit, which comprises the compound of formula (I) as a first compound, and a second compound selected from the group consisting of

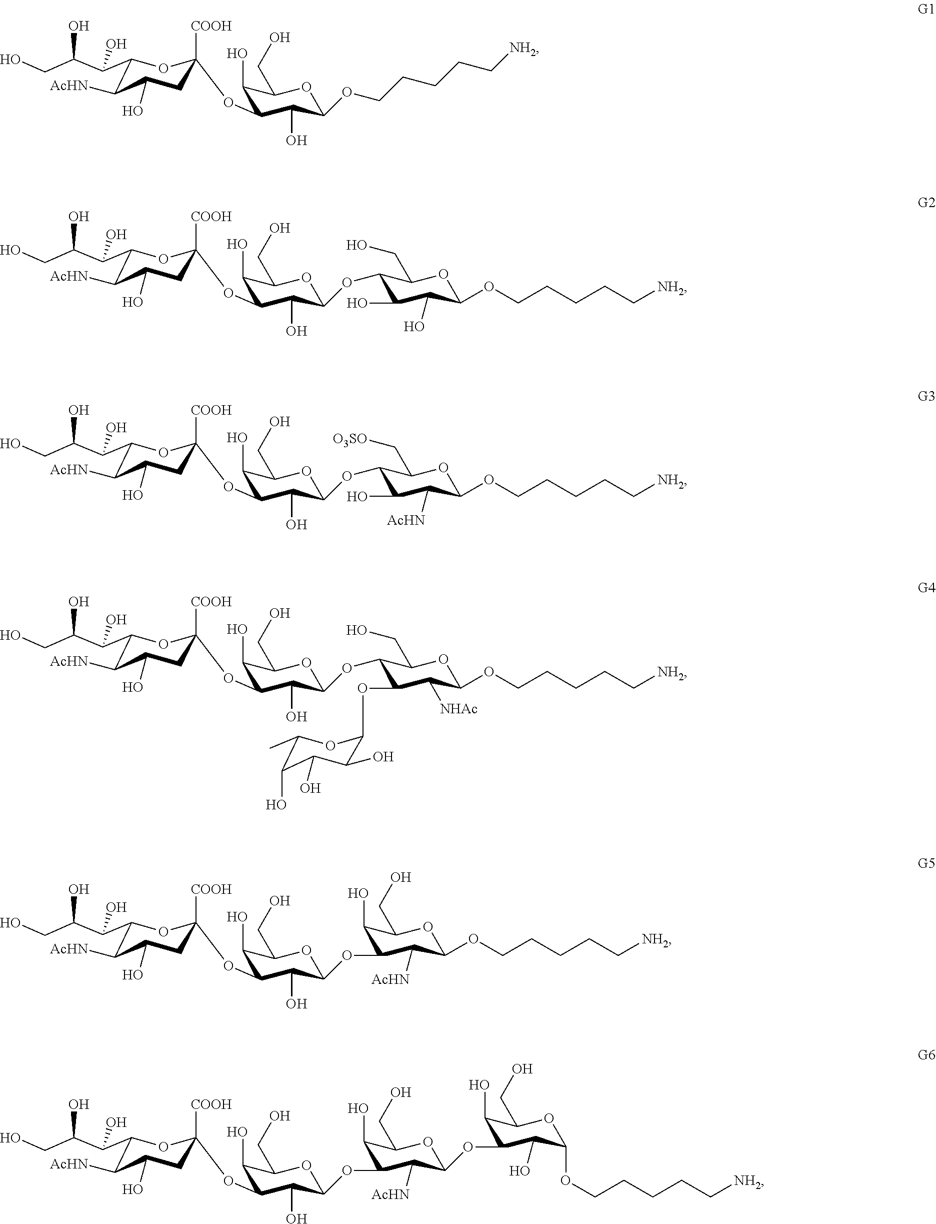

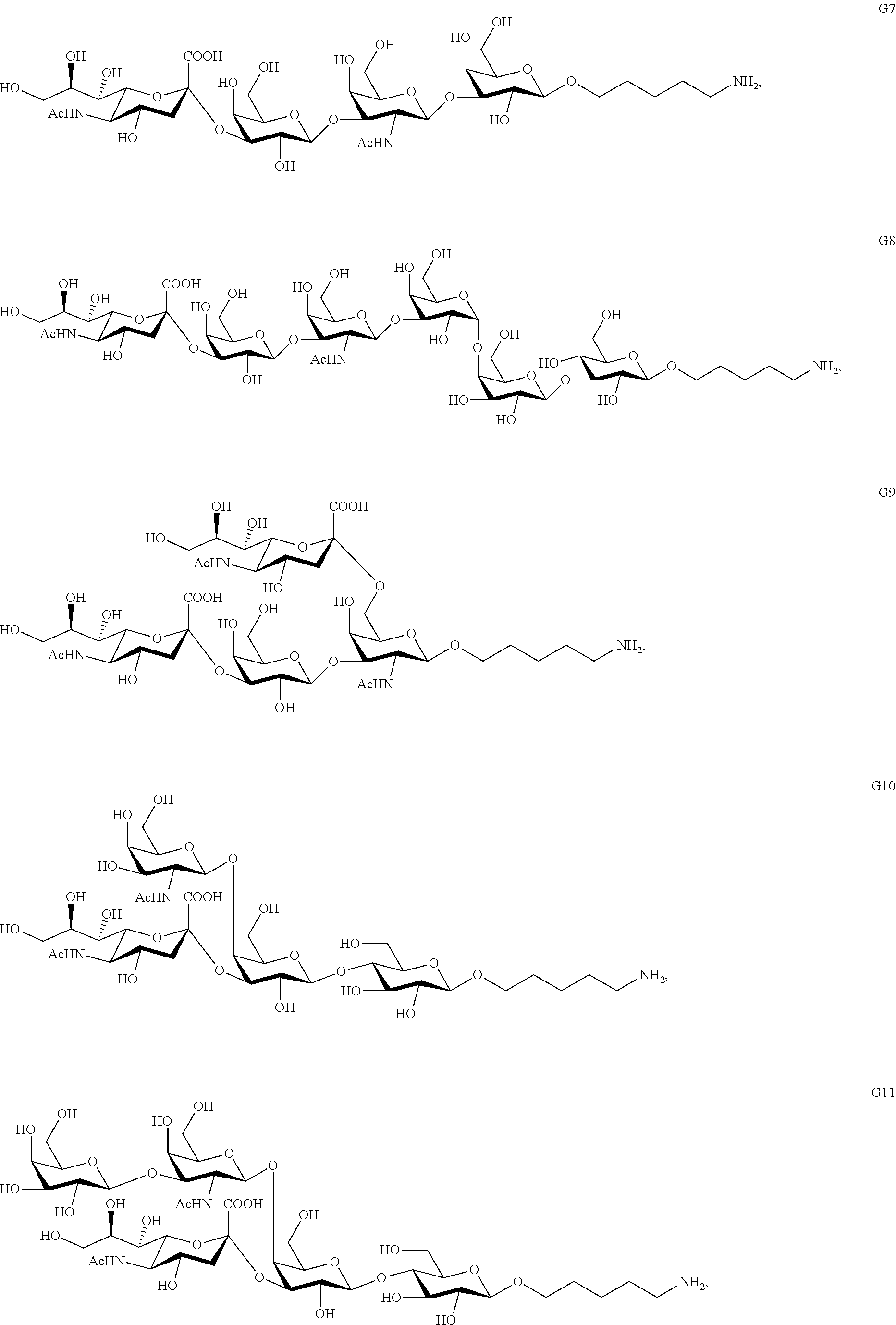

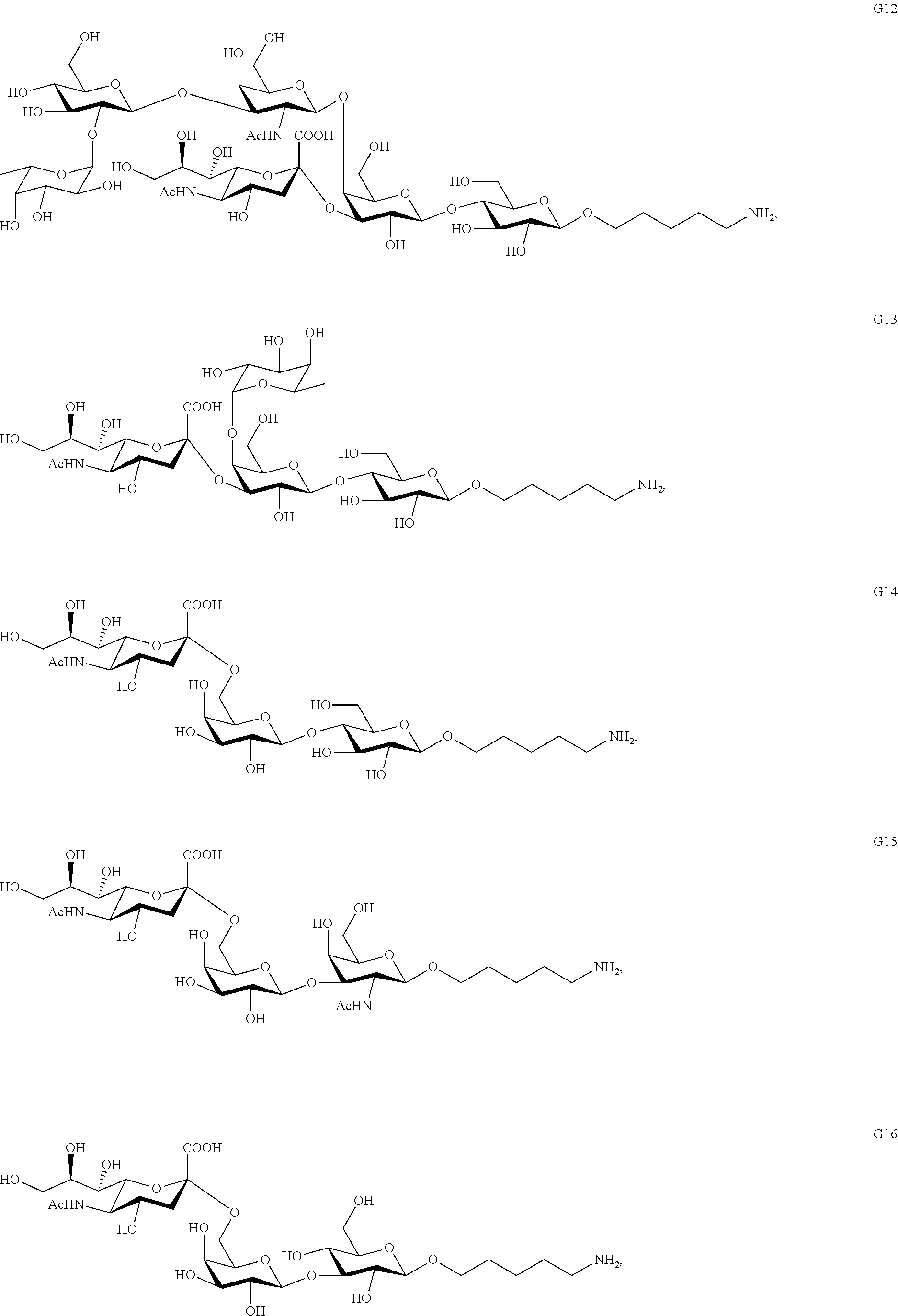

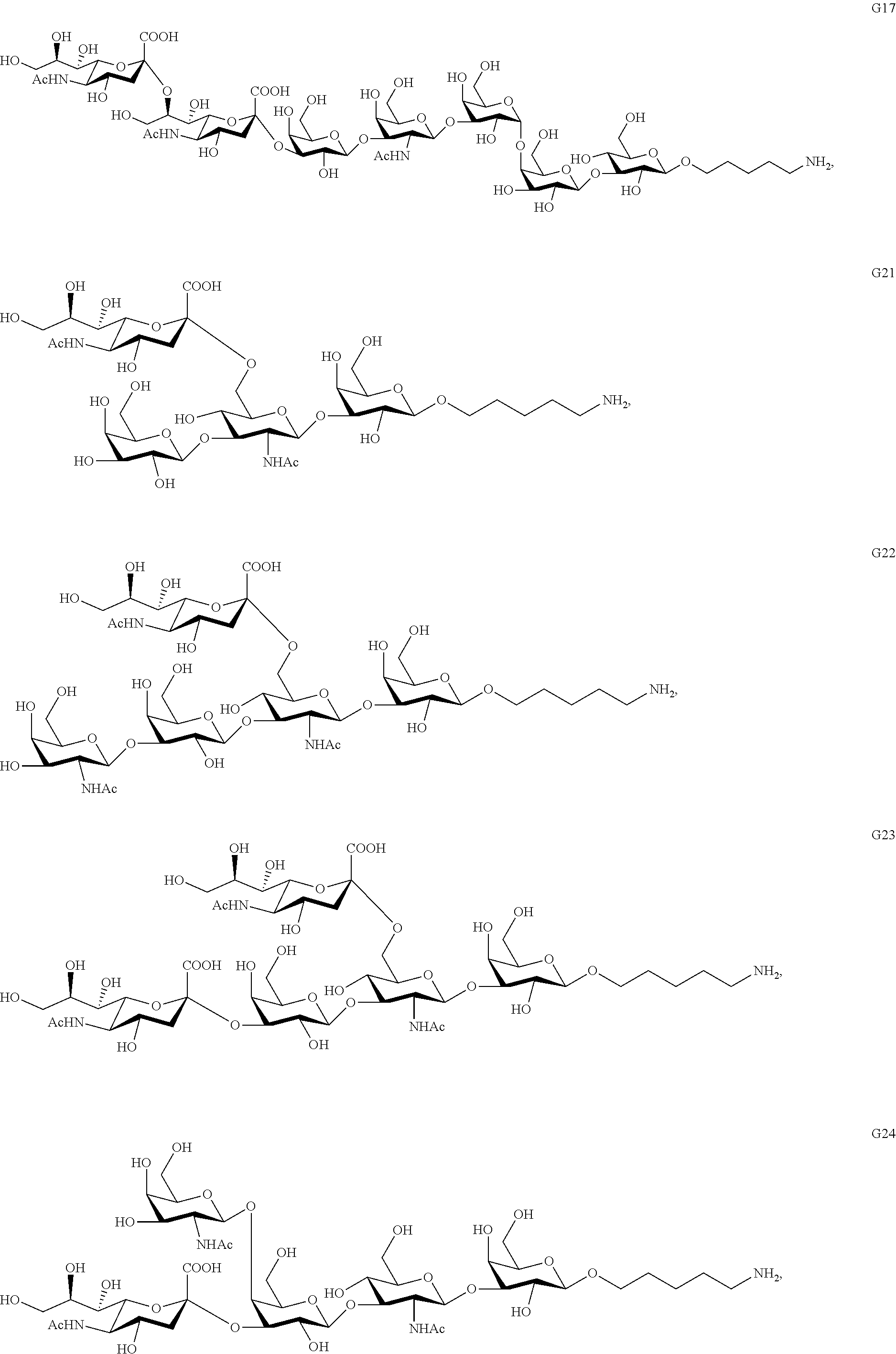

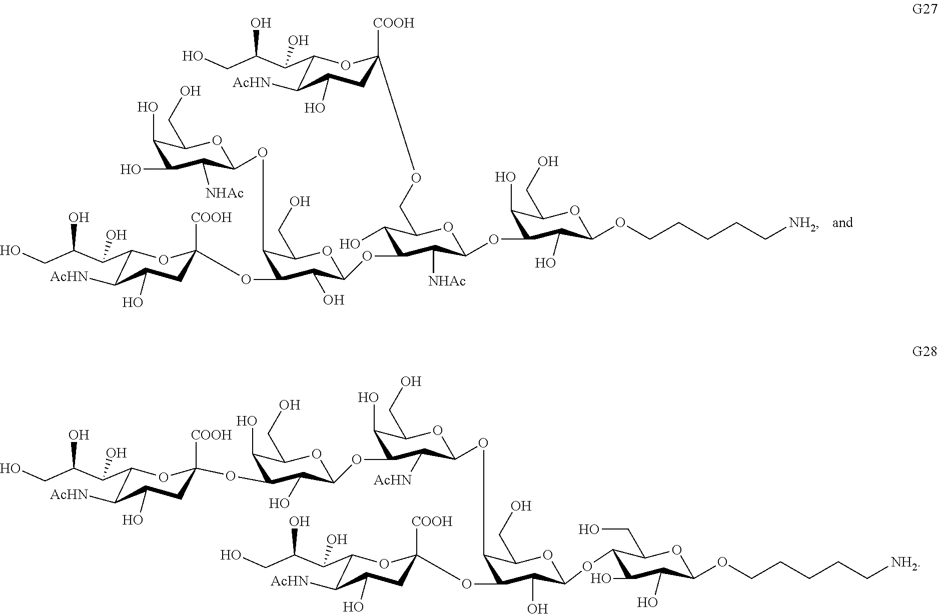

According to some embodiments of the present disclosure, the pharmaceutical kit is useful in distinguishing the subject having MCI from the healthy subject, in which the pharmaceutical kit comprises compound 1-1 (Gb19) as the first compound, and G3, G4, G9, G21, G23, G24 or G27 as the second compound. Alternatively, the pharmaceutical kit may comprises compound 1-2 (G25) as the first compound, and G3, G4, G9, G21, G23, G24 or G27 as the second compound so as to achieve the prognostic and/or diagnostic effect.

According to certain embodiments of the present disclosure, the pharmaceutical kit provides a means to identify the subject having MCI or AD; in these embodiments, the pharmaceutical kit comprises compound 1-3 (G18) as the first compound, and G9, G10, G11, G14, G21, G23, G24 or G27 as the second compound. Alternatively, the pharmaceutical kit may comprises compound 1-2 (G25) as the first compound, and G9, G10, G11, G14, G21, G23, G24 or G27 as the second compound to make the prognosis and/or diagnosis.

In some working examples, the present pharmaceutical kit for distinguishing the pre-HD subject from healthy subject comprises compound 1-5 (G20) as the first compound, and the G17 as the second compound.

According to certain embodiments, the present pharmaceutical kit for identifying pre-HD subject and HD patient comprises compound 1-5 (G20) as the first compound, and any of G1-G17, G21-G24 or G27-G28 as the second compound.

The pharmaceutical kit comprises two containers to respectively hold the first and second compound of the present disclosure, in which the containers may be formed from a variety of materials such as glass, or plastic. The kit may further comprise a label or package insert on or associated with the containers. The label or package insert indicates the use of the present pharmaceutical kit in evaluating the neurodegenerative diseases. Alternatively or additionally, the kit may further comprise a third container comprising a buffer or a diluent, such as a phosphate-buffered saline, Ringer's solution or dextrose solution. It may further include other materials desirable from a commercial and user standpoint, including other buffers, diluents, filters, plates, slides and tubes.

The third aspect of the present disclosure aims at providing a method for making a prognosis and/or diagnosis of a neurodegenerative diseases in a subject by use of the present compound or kit via analyzing a biological sample obtained from the subject. The method includes steps of:
(a) mixing the biological sample and the compound of formula (I) to form a first immunocomplex;
(b) reacting an anti-IgM antibody with the first immunocomplex of step (a) to give a second immunocomplex, wherein the anti-IgM antibody is conjugated with a reporter molecule;
(c) determining the signal level of the reporter of the step (b); and
(d) making the prognosis or diagnosis of the neurodegenerative disease based on the determination made in the step (c).

In general, the subject is a mammal; preferably, a human. The biological sample may be a whole blood sample, a serum sample, a plasma sample, or any other tissues or biological fluids that contain antibody (i.e., IgM, IgG, IgA, IgE and/or IgD antibody). According to one working example of the present disclosure, the biological sample is a plasma sample.

In the step (a), the biological sample (e.g., the plasma sample) is mixed with the compound of formula (I). After mixing, the compound of formula (I) would interact with the anti-ganglioside antibodies present in the biological sample thereby forming a first immunocomplex (i.e., a compound-antibody immunocomplex, in which the antibody may be in the form of IgM, IgG, IgA, IgE or IgD). Depending on the desired purposes, the compound of formula (I) may be first immobilized on the plate or slide followed by adding the biological sample to the plate. Alternatively, the compound of formula (I) may be suspended in a solution, and then mixed with the biological sample. Preferably, the unbound compound/antibody is removed before proceeding to the step (b).

In the step (b), the anti-IgM antibody conjugated with a reporter molecule is added to the first immunocomplex (i.e., the compound-antibody immunocomplex). The anti-IgM antibody may specifically recognize and bind to the IgM antibody portion of the first immunocomplex, and accordingly, forming a second immunocomplex (i.e., a compound-IgM antibody-anti-IgM antibody complex). Preferably, the unbound anti-IgM antibody is removed before proceeding to the step (c).

One skilled artisan may select suitable reporter molecule to be conjugated with the anti-IgM antibody. Non-limiting examples of the reporter molecule include, tag molecule, radioactive molecule, fluorescent molecule, phosphorescent molecule, chemiluminescent molecule and enzyme.

Then, the signal level emitted by the reporter is measured in the step (c). The method of measurement varies with the reporter molecule selected. For example, in the case when the reporter molecule is a fluorescent molecule, it may be detected by the flow cytometry or microarray scanner. Alternatively, when the reporter molecule is a chemiluminescent molecule, the chemiluminescence reader may be employed to detect the signal level emitted.

Based on the signal level, a practitioner or a person ordinarily skilled in the art may make a prognosis and/or diagnosis of the neurodegenerative disease as illustrated in the step (d). According to embodiments of the present disclosure, when the signal level is higher than that of a reference sample, then then subject has or is at risk of having the neurodegenerative disease. The reference sample may be derived from a healthy subject, or a database collecting various donor samples.

A practitioner may administer to a subject in need (e.g., a subject having a neurodegenerative disease) a treatment (e.g., an anti-degenerative treatment) in time based on the prognostic and/or diagnostic result. Thus, another aspect of the present disclosure is directed to a method of treating a neurodegenerative disease in a subject, comprising,
(a) obtaining a biological sample from the subject;
(b) mixing the biological sample of step (a) and the compound of formula (I) to form a first immunocomplex;
(c) reacting an anti-IgM antibody with the first immunocomplex of step (b) to give a second immunocomplex, wherein the anti-IgM antibody is conjugated with a reporter molecule;
(d) determining the signal level of the reporter of the step (c); and
(e) administering to the subject an effective amount of an anti-neurodegenerative treatment based on the determination made in the step (d), wherein the signal level in the biological sample of the subject is higher than that of a reference sample.

The steps (a) to (d) of the treating method are quite similar to the steps (a) to (c) of the prognostic/diagnostic method described above; hence, detailed description thereof is omitted herein for the sake of brevity.

In the step (e), the anti-neurodegenerative treatment is administered to the subject having a signal level higher than the signal level of the reference sample (e.g., a plasma sample of a healthy subject, or a database collecting various donor samples) thereby ameliorating and/or alleviating the symptoms associated with the neurodegenerative disease. Non-limiting examples of the anti-neurodegenerative treatment include, dopamine agonist (e.g., apomorphine. bromocriptine, lisurid, pergolid, dihydro-α-ergocryptine, cabergoline, rotigotine, pramipexol, ropinirol, piribedil, and levodopa), the inhibitor of monoaminooxidase B (MAO-B) (e.g., selegiline and rasagiline), the antagonist of N-Methyl D-Aspartate (NMDA) (e.g., amantadine and memantine), the antagonist of glutamate receptor (e.g., riluzole), anticholinergics (e.g., benztropine mesylate, biperiden, diphenhydramine, and trihexyphenidyl), antioxidant (e.g., curcumin, vitamin C, vitamin E, flavonoid, and polyphenols), the inhibitor of histone deacetylase (e.g., sodium butyrate, phenylbutyrate, and suberoylanilide hydroxamic acid), and a combination thereof.

The following Examples are provided to elucidate certain aspects of the present invention and to aid those of skilled in the art in practicing this invention. These Examples are in no way to be considered to limit the scope of the invention in any manner. Without further elaboration, it is believed that one skilled in the art can, based on the description herein, utilize the present invention to its fullest extent.

### EXAMPLES

### Materials

Commercial solvents and reagents were purchased from Sigma-Aldrich and Acros and used as received without further purification.

### General Methods

Molecular sieves 4 Å (Reidel-deHaen No. 31812) for glycosylations were crushed and activated by heating at 350° C. for 10 hours before use. Reactions were monitored with analytical TLC plates (PLC silica gel-60, F₂₅₄, 2 mm) and visualized under UV (254 nm) or by staining with acidic ceric ammonium molybdate or p-anisaldehyde. Flash column chromatography was performed on silica gel (40-63 μm), LiChroprep® RP8 (40-63 μm), and LiChroprep® RP18 (40-63 μm).

### Instruments

Proton nuclear magnetic resonance (¹H NMR) spectra and carbon nuclear magnetic resonance (¹³C NMR) spectra were recorded on a NMR spectrometer (600 MHz/150 MHz). Chemical shifts of protons were reported in ppm (δ scale) and referenced to tetramethylsilane (δ=0). Chemical shifts of carbon were also reported in parts per million (ppm, δ scale) and were calibrated with tetramethylsilane (δ=0). DEPT 135 (distortionless enhancement by polarization transfer) was employed for determination of multiplicity. Data were represented as follows: chemical shift, multiplicity (s=singlet, d=doublet, t=triplet, q=quartet, m=multiplet, br=broad), coupling constant (J) in Hz, and integration. High resolution mass spectra were obtained using BioTOF™ III, and MALDI-TOF MS were obtained using Ultraflex™ II TOF/TOF.

### Plasma Sample Collection

Human plasma samples were collected from healthy individuals, pre-HD cases, and HD patients. Samples were fully encoded to protect patient confidentiality.

Human plasma samples were obtained from healthy individuals, mild cognitive impairment (MCI), Alzheimer's disease (AD) patients. Samples were fully encoded to protect patient confidentiality. Plasma samples are distinguished normal from patients by several clinical diagnosis (such as patient history, physical exam, CT scan, magnetic resonance imaging).

### Glycan Microarray Fabrication

The fabrication was performed following previous procedure. Briefly, microarrays were printed by a robotic pin to deposit approximately 0.7 nl of 100 μM amine-containing glycans in the printing buffer containing 300 mM sodium phosphate buffer, pH 8.5, 0.05% Triton™ X-100 from a 96-well microtiter plate onto NHS-coated glass slides. Each glycan were spotted from bottom to top with 10 replicates and two kinds of glycans were spotted in one row that was horizontally placed in each subarray/grid. There were 28 different glycans spotted in each subarray/grid and one array slide contained 16 identical grids for different plasma samples. Printed slides were allowed to react in an atmosphere of 80% humidity for an hour followed by desiccation overnight. These slides were stored at room temperature in a desiccator until use. Before the binding assay, these slides were blocked with phosphate buffered saline (PBS) buffer, pH 7.4, containing 3% bovine serum albumin (BSA) and then washed with double distilled water and PBS buffer twice.

### Glycan Microarray Analysis of Plasma

The plasma samples from healthy individuals, pre-HD stage cases, and HD patients were diluted 1:100 with 0.05% Tween® 20/3% BSA/PBS buffer, pH 7.4, and applied to the grids on the glycan microarrays and then incubated in a humidifying chamber with shaking for 1 hour in a closed box. Then, the slides were washed three times each with PBS buffer, pH 7.4, containing 0.05% Tween® 20, PBS buffer, pH 7.4, and double distilled water. Next, Cy3-conjugated goat anti-human IgM antibody was added to the slide as described above and incubated in a humidifying chamber incubation with shaking under a coverlid for 1 hour. The slide was washed three times each with PBS buffer, pH 7.4, with 0.05% Tween® 20; PBS buffer, pH 7.4, with 0.05% Triton™ X-100; PBS buffer, pH 7.4; double distilled water and dried. The slide was scanned at 635 nm with a microarray fluorescence chip reader.

### Slot-Blotting

All plasma samples were diluted in TBS buffer (100 mM Tris-HCl, 150 mM NaCl, pH 7.4) before vacuum filtration through a 48-well dot blot apparatus containing NC membranes. The membranes were blocked in 10% milk dissolved in TBS buffer for 1 hour. Membranes were washed twice in TBS buffer. For analysis the total IgM levels, the membrane was incubated with HRP-linked anti-IgM secondary antibody for 1 hour. For analysis the fucosylated components levels, the membrane was incubated with lectins for 1 hour. Then, the membrane was washed twice in TBS buffer and treated with HRP-linked streptavidin secondary antibody for 1 hour. The membranes were washed again twice in TBS buffer, then developed with chemiluminescent substrate. The slots were quantified by software.

### Data Analysis

Software was used for the fluorescence analysis of the extracted data. The local background was subtracted for each antibody spot. The replicate spots were averaged in the same array. The table of demographic characterization was analyzed by Chi-square test for gender, unpaired Student t-test for CAG repeats, and one-way ANOVA for age by using SPSS program. A value of P<0.05 was considered to be statistically significant. The correlation of each 28 kinds of glycan and age were evaluated by Pearson. To identify potential glycan for detection of disease progression, odds ratios (ORs) and 95% confidence interval (CI) were calculated by using logistic regression. The selections of algorithm in SAS program were used with stepwise, forward, and backward. Receiver-operating characteristic (ROC) analyses were used to differentiate between normal control v.s. pre-HD cases and pre-HD cases v.s. HD patients. To evaluate the area under the curve (AUC) with the corresponding 95% CI was reported as well as sensitivity and specificity.

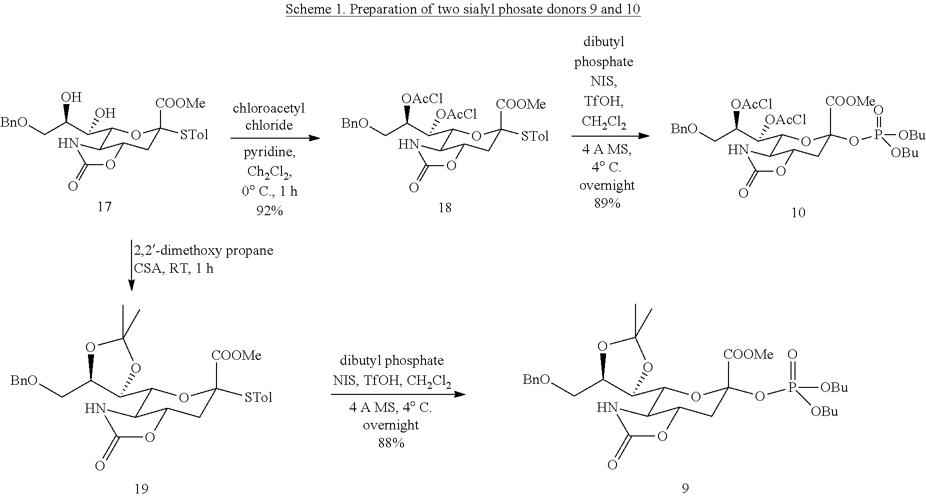

### Methyl (4-methylphenyl 5-amino-9-O-benzyl-5-N,4-O-carbonyl-7,8-di-O)-chloroacetyl-3,5-dideoxy-2-thio-D-glycero-α-D-galacto-2-nonulopyranosid)onate (18)

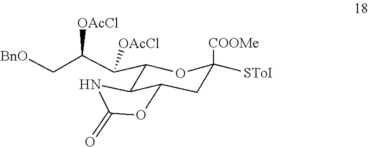

To a stirred solution of methyl (4-methylphenyl 5-amino-9-O-benzyl-5-N,4-O-carbonyl-3,5-dideoxy-2-thio-D-glycero-α-D-galacto-2-nonulopyranosid)onate (17) (2.00 g, 3.97 mmol, 1.00 eq.) in dry CH₂Cl₂ (80 mL) was added pyridine (4.82 mL, 59.59 mmol, 15.00 eq.) and chloroacetyl chloride (1.26 mL, 15.84 mmol, 4.00 eq.) at 0° C. under argon. After being stirred at 0° C. for 1 h, the reaction mixture was poured into 1 M aq. HCl. The aqueous phase was washed with two portions of CH₂Cl₂. The combined extracts were washed with saturated aq. NaHCO₃ and brine, dried over MgSO₄, filtered, and evaporated in vacuo. The residue was chromatographed on silica gel with 60:40 hexane-ethyl acetate to give methyl (4-methylphenyl 5-amino-9-O-benzyl-5-N4-O-carbonyl-7,8-di-O-chloroacetyl-3,5-dideoxy-2-thio-D-glycero-α-D-galacto-2-nonulopyranosid)onate (18) (2.39 g, 3.64 mmol, 92%). ¹H NMR (600 MHz, CDCl₃) δ 7.23-7.37 (m, 7H), 7.12 (d, 2H, J=8.1 Hz), 5.38 (dd, 1H, J=9.8, 1.6 Hz), 5.34 (br-s, 1H), 5.30 (dt, 1H, J=9.8, 2.3 Hz), 4.58 (d, 1H, J=12.0 Hz), 4.34 (d, 1H, J=12.0 Hz), 4.15 (d, 1H, J=15.3 Hz), 4.04 (d, 1H, J=15.4 Hz), 4.03 (dd, 1H, J=9.9, 1.7 Hz), 3.95 (d, 1H, J=15.0 Hz), 3.86 (ddd, 1H, J=13.5, 10.0, 3.6 Hz), 3.78 (d, 1H, J=14.8 Hz), 3.74 (dd, 1H, J=11.4, 2.0 Hz), 3.61 (dd, 1H, J=11.5, 2.7 Hz), 3.55 (s, 3H), 3.06 (dd, 1H, J=12.0, 3.7 Hz), 2.96 (ddd, 1H, J=10.4, 10.3, 1.4 Hz), 2.34 (s, 3H), 2.09 (t, 12.4 Hz); ¹³C NMR (150 MHz, CDCl₃) δ 167.99, 167.96, 166.44, 159.06, 140.78, 137.23, 136.25, 130.02, 128.81, 128.49, 128.40, 124.81, 88.53, 77.61, 75.11, 73.59, 70.75, 70.54, 66.58, 57.85, 53.24, 41.18, 40.46, 37.70, 21.53; HRMS (ESI-TOF) Calcd for C₂₉H₃₁NO₁₀SCl₂Na [M+Na]⁺ 678.0943, found 678.0941.

### Methyl (5-amino-9-O-benzyl-5-N4-O-carbonyl-7,8-di-O-chloroacetyl-2-(dibutylphosphoryl)-3,5-dideoxy-D-glycero-α-D-galacto-2-nonulopyranosid)onate (10)

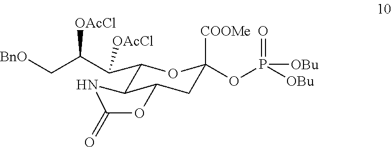

To a stirred solution of methyl (4-methylphenyl 5-amino-9-O-benzyl-5-N4-O-carbonyl-7,8-di-O-chloroacetyl-3,5-dideoxy-2-thio-D-glycero-α-D-galacto-2-nonulopyranosid)onate (18) (2.15 g, 3.27 mmol, 1.00 eq.), dibutyl phosphate (1.83 mL, 9.84 mmol, 3.00 eq.) and pulverized activated MS-4 Å in dry CH₂Cl₂ (80 mL) was added N-iodosuccinimide (1.47 g, 6.53 mmol, 2.00 eq.) and 0.5 M trifluoromethanesulfonic acid solution in dry Et₂O (1.96 mL, 0.98 mmol, 0.30 eq.) at 0° C. under argon. After being stirred at the same temperature overnight, the reaction mixture was diluted with CH₂Cl₂ and filtered through a pad of celite. The filtrate was poured into a mixture of saturated aq. NaHCO₃ and saturated aq. Na₂S₂O₃. The aqueous layer was extracted with two portions of CH₂Cl₂. The combined extracts were washed with saturated aq. NaHCO₃ and saturated aq. Na₂S₂O₃ and brine, dried over MgSO₄, filtered, and evaporated in vacuo. The residue was chromatographed on silica gel with 60:40 hexane-ethyl acetate to give methyl (5-amino-9-O-benzyl-5-N4-O-carbonyl-7, 8-di-O-chloroacetyl-2-(dibutylphosphoryl)-3,5-dideoxy-D-glycero-α-D-galacto-2-nonulopyranosid)onate (10) (2.16 g, 2.91 mmol, 89%). ¹H NMR (600 MHz, CDCl₃) δ 7.21-7.35 (m, 5H), 5.39 (dd, 1H, J=9.8, 1.6 Hz), 5.38 (br-s, 1H), 5.32 (dt, 1H, J=9.8, 2.1 Hz), 4.57 (d, 1H, J=12.1 Hz), 4.45 (dd, 1H, J=10.0, 1.6 Hz), 4.34 (d, 1H, J=12.1 Hz), 4.26 (d, 1H, J=15.2 Hz), 4.14 (d, 1H, J=15.3 Hz), 3.96-4.07 (m, 6H), 3.82 (d, 1H, J=14.9 Hz), 3.79 (s, 3H), 3.66 (dd, 1H, J=11.3, 1.8 Hz), 3.55 (dd, 1H, J=11.3, 2.8 Hz), 3.23 (t, 1H, J=10.1 Hz), 2.86 (dd, 1H, J=12.2, 3.7 Hz), 2.61 (t, 1H, J=12.8 Hz), 1.58-1.63 (m, 4H), 1.32-1.39 (m, 4H), 0.891 (t, 3H, J=7.5 Hz), 0.889 (t. 3H, J=7.4 Hz); ¹³C NMR (150 MHz, CDCl₃) δ 168.1, 167.7 (d, 7.5 Hz, ³J_{C-Hax(}2.59)=6.0 Hz), 166.4, 159.2, 137.2, 128.8, 128.4, 98.9 (d, 7.5 Hz), 76.1, 75.4, 73.6, 70.22, 70.16, 68.2 (d, 6.0 Hz), 68.1 (d, 6.0 Hz), 66.6, 57.2, 53.7, 41.1, 40.5, 37.3 (d, 4.4 Hz), 32.3 (d, 6.0 Hz), 32.2 (d, 6.0 Hz), 18.79, 18.76, 13.8, 13.7; HRMS (ESI-TOF) Calcd for C₃₀H₄₂NO₁₄PCl₂Na [M+Na]⁺ 794.1618, found 794.1614.

### Methyl (4-methylphenyl 5-amino-9-O-benzyl-5-N4-O-carbonyl-3,5-dideoxy-7,8-O-isopropylidene-2-thio-D-glycero-α-D-galacto-2-nonulopyranosid)onate (19)

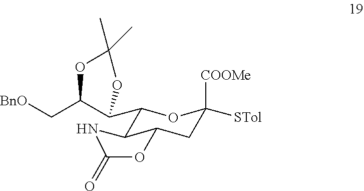

To a stirred solution of methyl (4-methylphenyl 5-amino-9-O-benzyl-5-N4-O-carbonyl-3,5-dideoxy-2-thio-D-glycero-α-D-galacto-2-nonulopyranosid)onate (17) (2.42 g, 4.81 mmol, 1.00 eq.) in 2,2′-dimethoxypropane (30 mL) was added 10-camphorsulfonic acid (0.67 g, 2.88 mmol, 0.60 eq.) at room temperature under argon. After being stirred at room temperature for 1 h, the reaction mixture was neutralized with triethylamine and evaporated in vacuo. The residue was chromatographed on silica gel with 70:30 hexane-ethyl acetate to give methyl (4-methylphenyl 5-amino-9-O-benzyl-5-N4-O-carbonyl-3,5-dideoxy-7,8-O-isopropylidene-2-thio-D-glycero-α-D-galacto-2-nonulopyranosid)onate (19) (2.36 g, 4.34 mmol, 90%). ¹H NMR (600 MHz, CDCl₃) δ 7.52 (d, 2H, J=8.1 Hz), 7.23-7.31 (m, 5H), 7.14 (d, 2H, J=8.0 Hz), 5.17 (br-s, 1H), 4.47 (dd, 1H, J=12.6, 6.5 Hz), 4.46 (d, 1H, J=11.9 Hz), 4.31 (d, 1H, J=12.0 Hz), 4.00 (d, 1H, J=6.7 Hz), 3.86-3.91 (m, 1H), 3.73-3.78 (m, 2H), 3.63-3.68 (m, 2H), 3.54 (s, 3H), 3.17 (dd, 1H, J=11.8, 3.5 Hz), 2.32 (s, 3H), 2.17 (t, 1H, J=12.1 Hz), 1.71 (s, 3H), 1.39 (s, 3H); ¹³C NMR (150 MHz, CDCl₃) δ 168.5, 159.6, 140.6, 138.3, 137.1, 129.8, 128.6, 128.1, 127.9, 125.4, 110.5, 89.1, 77.8, 77.1, 76.3, 75.9, 73.2, 67.9, 58.1, 52.9, 37.6, 26.7, 25.9, 21.6; HRMS (ESI-TOF) Calcd for C₂₈H₃₃NO₈SNa [M+Na]⁺ 566.1825, found 566.1821.

### Methyl (5-amino-9-O-benzyl-5-N4-O-carbonyl-2-(dibutylphosphoryl)-3,5-dideoxy-7,8-O-isopropylidene-D-glycero-α-D-galacto-2-nonulopyranosid)onate (9)

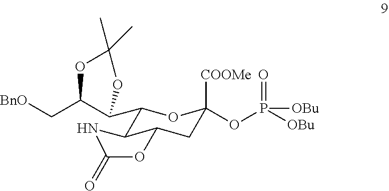

To a stirred solution of methyl (4-methylphenyl 5-amino-9-O-benzyl-5-N4-O-carbonyl-3,5-dideoxy-7,8-O-isopropylidene-2-thio-D-glycero-α-D-galacto-2-nonulopyranosid)onate (19) (2.80 g, 5.15 mmol, 1.00 eq.), dibutyl phosphate (2.87 mL, 15.43 mmol, 3.00 eq.) and pulverized activated MS-4 Å in dry CH₂Cl₂ (110 mL) was added N-iodosuccinimide (2.32 g, 10.31 mmol, 2.00 eq.) and 0.5 M trifluoromethanesulfonic acid solution in dry Et₂O (3.09 mL, 1.55 mmol, 0.30 eq.) at 0° C. under argon. After being stirred at the same temperature overnight, the reaction mixture was diluted with CH₂Cl₂ and filtered through a pad of celite. The filtrate was poured into a mixture of saturated aq. NaHCO₃ and saturated aq. Na₂S₂O₃. The aqueous layer was extracted with two portions of CH₂Cl₂. The combined extracts were washed with saturated aq. NaHCO₃ and saturated aq. Na₂S₂O₃ and brine, dried over MgSO₄, filtered, and evaporated in vacuo. The residue was chromatographed on silica gel with 60:40 hexane-ethyl acetate to give Methyl (5-amino-9-O-benzyl-5-N4-O-carbonyl-2-(dibutylphosphoryl)-3,5-dideoxy-7,8-O-isopropyliden e-D-glycero-α-D-galacto-2-nonulopyranosid)onate (9) (2.85 g, 4.53 mmol, 88%). ¹H NMR (600 MHz, CDCl₃) δ 7.25-7.35 (m, 5H), 5.34 (br-s, 1H), 4.59 (s, 2H), 4.47 (dd, 1H, J=12.6, 6.5 Hz), 4.32 (dd, 1H, J=9.7, 1.6 Hz), 3.97-4.13 (m, 8H), 3.80 (s, 3H), 3.65 (t, 1H, J=10.5 Hz), 2.95 (dd, 1H, J=11.8, 3.5 Hz), 2.33 (t, 1H, J=12.8 Hz), 1.60-1.67 (m, 4H), 1.47 (s, 3H), 1.35-1.42 (m, 4H), 1.33 (s, 3H), 0.92 (t, 3H, J=7.4 Hz), 0.90 (t. 3H, J=7.4 Hz); ¹³C NMR (150 MHz, CDCl₃) δ 168.1, 159.7, 138.4, 128.6, 128.3, 127.9, 109.7, 99.6 (d, 6.2 Hz), 76.5, 76.4, 75.6, 73.6, 68.8, 68.5 (d, 5.7 Hz), 68.1 (d, 6.2 Hz), 58.0, 53.4, 38.4 (d, 8.0 Hz), 32.34 (d, 7.6 Hz), 32.28 (d, 7.6 Hz), 26.6, 25.3, 18.8, 13.8; HRMS (ESI-TOF) Calcd for C₂₉H₄₄NO₁₂PNa [M+Na]⁺ 652.2499, found 652.2493.

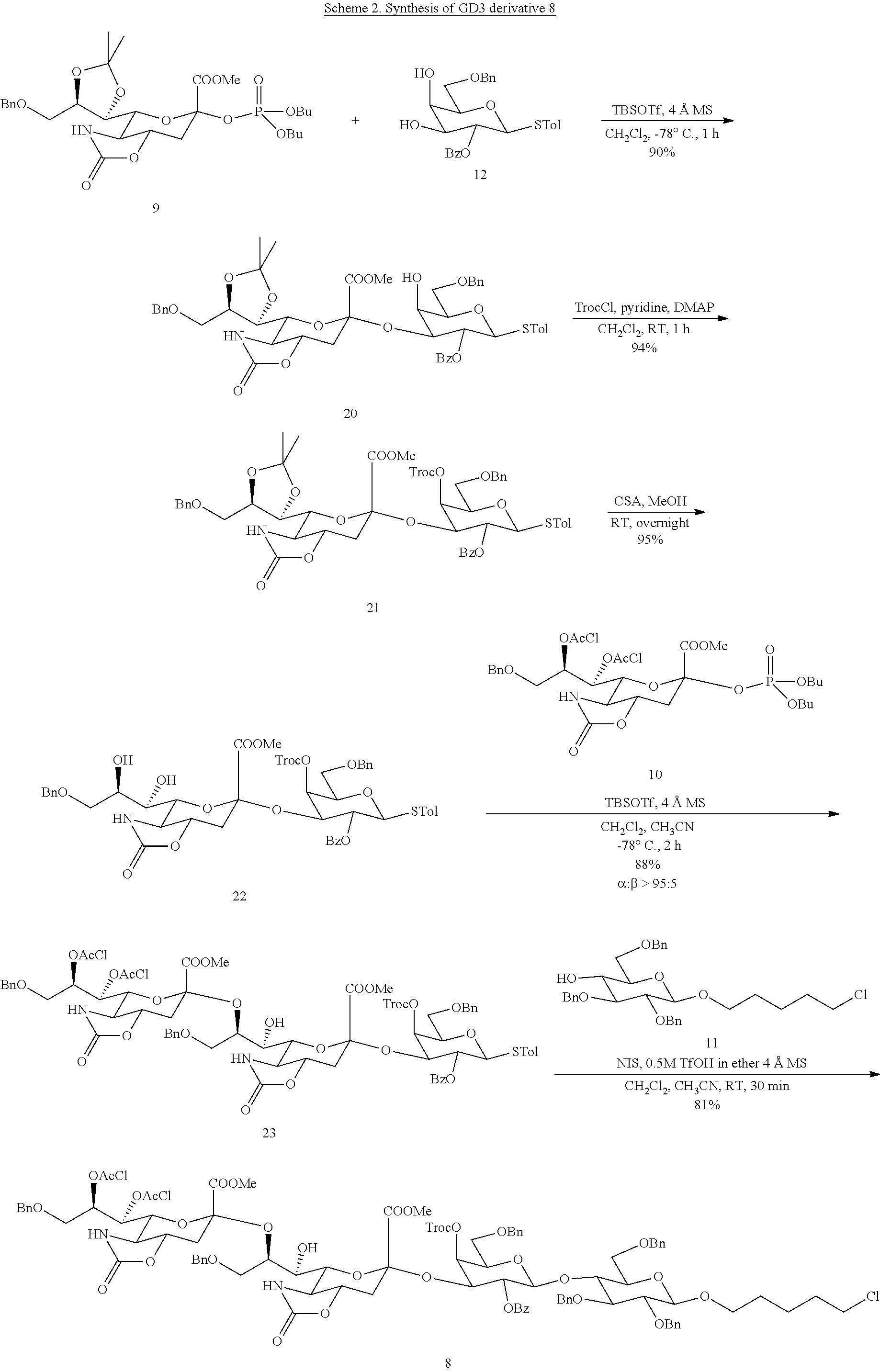

### 4-methylphenyl 2-O-benzoyl-6-O-benzyl-3-O-(methyl 5-amino-9-O-benzyl-5-N4-O-carbonyl-3,5-dideoxy-7,8-O-isopropylidene-D-glycero-α-D-galacto-2-nonulopyranoylonate)-1-thio-α-D-galactopyranoside (20)

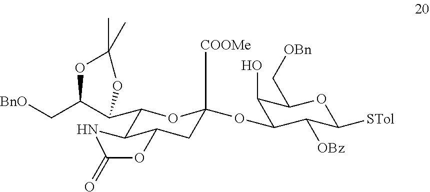

To a stirred solution of Methyl (5-amino-9-O-benzyl-5-N4-O-carbonyl-2-(dibutylphosphoryl)-3,5-dideoxy-7,8-O-isopropylidene-D-glycero-α-D-galacto-2-nonulopyranosi d)onate (9) (2.12 g, 3.37 mmol, 1.20 eq.), 4-methylphenyl 2-O-benzoyl-6-O-benzyl-1-thio-β-D-galactopyranoside (12) (1.35 g, 2.81 mmol, 1.00 eq.) and pulverized activated MS-4 Å in dry CH₂Cl₂ (50 mL) was added TBSOTf (0.93 mL, 4.05 mmol, 1.40 eq.) at −78° C. under argon. After being stirred at the same temperature for 1 h, the reaction mixture was neutralized with triethylamine and filtered through a pad of celite. The filtrate mixture was poured into a mixture of saturated aq. NaHCO₃ and saturated aq. Na₂S₂O₃. The aqueous layer was extracted with two portions of CH₂Cl₂. The combined extracts were washed with brine, dried over MgSO₄, filtered, and evaporated in vacuo. The residue was chromatographed on silica gel with 60:40 hexane-ethyl acetate to give 4-methylphenyl 2-O-benzoyl-6-O-benzyl-3-O-(methyl-5-amino-9-O-benzyl-5-N4-O-carbonyl-3,5-dideoxy-7,8-O-isopropylidene-D-glycero-α-D-galacto-2-nonulo pyranoylonate)-1-thio-β-D-galactopyranoside (20) (2.31 g, 2.57 mmol, 90%, a only). The α/β ratio was determined by ¹H NMR analysis. ¹H NMR (600 MHz, CDCl₃) δ 8.02 (dd, 2H, J=7.7, 1.3 Hz), 7.58 (t, 1H, J=7.5 Hz), 7.45 (t, 2H, J=7.8 Hz), 7.34 (d, 2H, J=8.1 Hz), 7.25-7.31 (m, 10H), 7.01 (d, 2H, J=8.1 Hz), 5.42 (t, 1H, J=9.8 Hz), 5.30 (br-s, 1H), 4.69 (d, 1H, J=10.1 Hz), 4.55 (dd, 2H, J=14.6, 11.8 Hz), 4.51 (t, 2H, J=12.8 Hz), 4.44 (dt, 1H, J=5.1, 6.8 Hz), 4.30 (dd, 1H, J=9.5, 3.1 Hz), 4.13 (d, 1H, J=2.9 Hz), 4.03 (dd, 1H, J=7.1, 2.0 Hz), 3.92 (ddd, 1H, J=12.8, 11.4, 3.6 Hz), 3.87 (d, 1H, J=9.9 Hz), 3.86 (dd, 1H, J=10.0, 3.1 Hz), 3.75-3.82 (m, 3H), 3.67 (d, 1H, J=5.5 Hz), 3.66 (s, 3H), 3.43 (t, 1H, J=10.8 Hz), 2.60 (dd, 1H, J=12.0, 3.6 Hz), 2.27 (s, 3H), 1.95 (t, 1H, J=12.5 Hz), 1.41 (s, 3H), 1.30 (s, 3H); ¹³C NMR (150 MHz, CDCl₃) δ 168.7, 165.3, 159.7, 138.27, 138.25, 138.0, 133.6, 133.2, 129.9, 129.81, 129.78, 129.2, 128.8, 128.7, 128.6, 128.1, 128.0, 127.9, 109.2, 100.0, 87.2, 77.6, 76.9, 76.3, 75.7, 75.4, 75.2, 73.8, 73.7, 69.6, 68.9, 68.7, 68.6, 58.3, 53.5, 36.6, 27.1, 24.7, 21.3; HRMS (ESI-TOF) Calcd for C₄₈H₅₃NO₁₄SNa [M+Na]⁺ 922.3084, found 922.3091.

### 4-methylphenyl 2-O-benzoyl-6-)-benzyl-3-O-(methyl 5-amino-9-O-benzyl-5-N4-O-carbonyl-3,5-dideoxy-7,8-O-isopropylidene-D-glycero-α-D-galacto-2-nonulopyranoylonate)-4-O-(2,2,2-trichloroethoxycarbonyl)-1-thio-β-D-galactopyranoside (21)

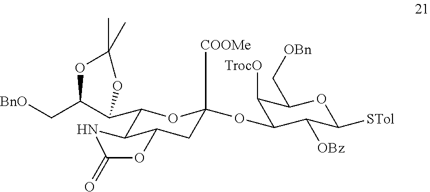

To a stirred solution of 4-methylphenyl 2-O-benzoyl-6-)-benzyl-3-O-(methyl 5-amino-9-O-benzyl-5-N4-O-carbonyl-3,5-dideoxy-7,8-)-isopropylidene-D-glycero-α-D-galacto-2-nonulopyranoylonate)-1-thio-β-D-galactopyranoside (20) (2.26 g, 2.51 mmol, 1.00 eq.) in dry CH₂Cl₂ (35 mL) was added dry pyridine (0.61 mL, 7.54 mmol, 3.00 eq.), 4-dimethylaminopyridine (31 mg, 2.54 mmol, 0.10 eq.) and 2,2,2-trichloroethyl chloroformate (0.69 mL, 5.01 mmol, 2.00 eq.) at room temperature under argon. After being stirred at the same temperature for 1 h, the reaction mixture was poured into 1 M HCl with cooling. The aqueous layer was extracted with two portions of CH₂Cl₂. The combined extracts were washed with 1 M aq. HCl, saturared aq. NaHCO₃ and brine, dried over MgSO₄, filtered, and evaporated in vacuo. The residue was chromatographed on silica gel with 60:40 hexane-ethyl acetate to give 4-methylphenyl 2-O-benzoyl-6-O-benzyl-3-O-(methyl 5-amino-9-O-benzyl-5-N, 4-O-carbonyl-3,5-dideoxy-7,8-O-isopropylidene-D-glycero-α-D-galacto-2-nonulopyranoylonate)-4-O-(2,2,2-trichloroethoxycarbonyl)-1-thio-β-D-galactopyranoside (21) (2.54 mg, 2.36 mmol, 94%). ¹H NMR (600 MHz, CDCl₃) δ 8.06 (dd, 2H, J=8.3, 1.2 Hz), 7.59 (t, 1H, J=7.5 Hz), 7.46 (t, 2H, J=7.7 Hz), 7.34 (d, 2H, J=8.2 Hz), 7.22-7.31 (m, 10H), 7.02 (d, 2H, J=8.2 Hz), 5.47 (t, 1H, J=10.0 Hz), 5.40 (d, 1H, J=2.9 Hz), 5.32 (br-s, 1H), 4.78 (d, 1H, J=11.8 Hz), 4.73 (d, 1H, J=10.0 Hz), 4.64 (d, 1H, J=11.8 Hz), 4.58 (d, 1H, J=11.9 Hz), 4.55 (d, 1H, J=11.9 Hz), 4.48 (dd, 1H, J=10.0, 2.9 Hz), 4.47 (s, 2H), 4.42 (dd, 1H, J=12.8, 5.9 Hz), 4.02 (dd, 1H, J=7.1, 2.2 Hz), 3.92 (d, 2H, J=5.9 Hz), 3.84 (ddd, 1H, J=12.8, 11.4, 3.5 Hz), 3.80 (d, 1H, J=6.1 Hz), 3.78 (dd, 1H, J=9.6, 2.4 Hz), 3.67 (dd, 1H, J=10.2, 6.5 Hz), 3.66 (s, 3H), 3.60 (dd, 1H, J=9.9, 5.9 Hz), 3.38 (t, 1H, J=10.1 Hz), 2.55 (dd, 1H, J=12.0, 3.5 Hz), 2.28 (s, 3H), 1.90 (t, 1H, J=12.5 Hz), 1.44 (s, 3H), 1.31 (s, 3H); ¹³C NMR (150 MHz, CDCl₃) δ 167.5, 165.1, 159.7, 153.8, 138.4, 138.2, 137.9, 133.8, 133.2, 130.1, 129.9, 129.6, 129.2, 128.8, 128.61, 128.56, 128.1, 127.95, 127.91, 109.0, 100.4, 94.7, 87.8, 76.6, 76.4, 75.4, 75.3, 74.9, 73.9, 73.5, 72.9, 68.9, 68.8, 68.5, 58.5, 53.4, 36.5, 27.5, 24.7, 21.4; HRMS (ESI-TOF) Calcd for C₅₁H₅₄NO₁₆SCl₃Na [M+Na]⁺ 1096.2127, found 1096.2113.

### 4-methylphenyl 2-O-benzoyl-6-O-benzyl-3-O-(methyl 5-amino-9-O-benzyl-5-N4-O-carbonyl-3,5-dideoxy-D-glycero-α-D-galacto-2-nonulopyranoylonate)-4-O-(2,2,2-trichloroethoxycarbonyl)-1-thio-β-D-galactopyranoside (22)

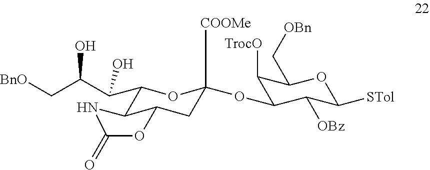

To a stirred solution of 4-methylphenyl 2-O-benzoyl-6-O-benzyl-3-O-(methyl 5-amino-9-O-benzyl-5-N4-O-carbonyl-3,5-dideoxy-7,8-O-isopropylidene-D-glycero-α-D-galacto-2-nonulopyranoylonate)-4-O-(2,2,2-trichloroethoxycarbonyl)-1-thio-β-D-galactopyranoside (21) (1.24 g, 1.15 mmol, 1.00 eq.) in Methanol (35 mL) was added 10-camphorsulfonic acid (0.27 g, 1.16 mmol, 1.00 eq.) at room temperature. After being stirred at room temperature overnight, the reaction mixture was neutralized with triethylamine and evaporated in vacuo. The residue was chromatographed on silica gel with 60:40 hexane-ethyl acetate to give 4-methylphenyl 2-O-benzoyl-6-O-benzyl-3-O-(methyl 5-amino-9-O-benzyl-5-N4-O-carbonyl-3,5-dideoxy-D-glycero-α-D-galacto-2-nonulopyranoylonate)-4-O-(2,2,2-trichloroethoxycarbonyl)-1-thio-β-D-galactopyranoside (22) (1.13 g, 1.09 mmol, 95%). ¹H NMR (600 MHz, CDCl₃) δ 8.00 (d, 2H, J=7.4 Hz), 7.51 (t, 1H, J=7.5 Hz), 7.26-7.39 (m, 14H), 7.04 (d, 2H, J=8.0 Hz), 5.80 (br-s, 1H), 5.21 (d, 1H, J=3.1 Hz), 4.80 (d, 1H, J=11.8 Hz), 4.65 (d, 1H, J=11.9 Hz), 4.54 (s, 2H), 4.47 (d, 1H, J=11.8 Hz), 4.45 (d, 1H, J=11.5 Hz), 4.40-4.57 (m, 2H), 3.87 (t, 1H, J=6.4 Hz), 3.83 (ddd, 1H, J=13.2, 11.1, 3.6 Hz), 3.75 (s, 3H), 3.65-3.77 (m, 4H), 3.58 (dd, 1H, J=9.7, 6.9 Hz), 3.50-3.53 (m, 2H), 3.11 (br-s, 1H), 3.05 (t, 1H, J=9.7 Hz), 2.68 (dd, 1H, J=11.8, 3.2 Hz), 2.29 (s, 3H), 1.98 (t, 1H, J=12.5 Hz); ¹³C NMR (150 MHz, CDCl₃) δ 167.9, 159.5, 154.2, 138.6, 137.83, 137.75, 133.7, 133.4, 129.9, 128.9, 128.8, 128.6, 128.3, 128.1, 128.0, 127.9, 99.6, 94.6, 79.2, 77.6, 76.0, 74.7, 73.9, 73.8, 73.3, 71.3, 69.3, 68.0, 58.4, 53.7, 35.4, 21.4; HRMS (ESI-TOF) Calcd for C₄₈H₅₀NO₁₆SCl₃Na [M+Na]⁺ 1056.1814, found 1056.1812.

### 4-methylphenyl 2-O-benzoyl-6-O-benzyl-3-O-(methyl 5-amino-9-O-benzyl-5-N4-O-carbonyl-3,5-dideoxy-8-O-(methyl 5-amino-9-O-benzyl-5-N4-O-carbonyl-7,8-di-O-chloroacetyl-3,5-dideoxy-D-glycero-α-D-galacto-2-nonulopyranoylonate)-D-glycero-α-D-galacto-2-nonulopyranoylonate)-4-O-(2,2,2-trichloroethoxycarbonyl)-1-thio-β-D-galactopyranoside (23)

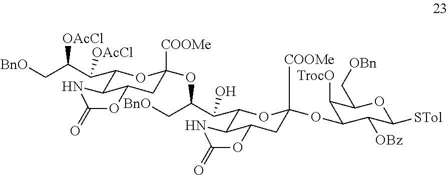

To a stirred solution of 4-methylphenyl 2-O-benzoyl-6-O-benzyl-3-O-(methyl 5-amino-9-O-benzyl-5-N4-O-carbonyl-3,5-dideoxy-D-glycero-α-D-galacto-2-nonulopyranoylonate)-4-O-(2,2,2-trichloroethoxycarbonyl)-1-thio-α-D-galactopyranoside (22) (2.89 g, 2.79 mmol, 1.00 eq.), methyl (5-amino-9-O-benzyl-5-N4-O-carbonyl-7,8-di-O-chloroacetyl-2-(dibutylphosphoryl)-3,5-dideoxy-D-glycero-α-D-galacto-2-nonulopyranosid)onate (10) (4.14 g, 5.58 mmol, 2.00 eq.) and pulverized activated MS-4 Å in a mixture of dry CH₂Cl₂ (36 mL) and acetonitrile (24 mL) was added TMSOTf (1.11 mL, 6.12 mmol, 2.20 eq.) at −78° C. under argon. After being stirred at the same temperature for 2 h, the reaction mixture was neutralized with saturated aq. NaHCO₃ and filtered through a pad of celite. The filtrate mixture was poured into a mixture of saturated aq. NaHCO₃. The aqueous layer was extracted with two portions of CH₂Cl₂. The combined extracts were washed with brine, dried over MgSO₄, filtered, and evaporated in vacuo. The residue was chromatographed on silica gel with 60:40 hexane-ethyl acetate to give 4-methylphenyl 2-O-benzoyl-6-O-benzyl-3-O-(methyl 5-amino-9-O-benzyl-5-N4-O-carbonyl-3,5-dideoxy-8-O-(methyl 5-amino-9-O-benzyl-5-N4-O-carbonyl-7,8-di-O-chloroacetyl-3,5-dideoxy-D-glycero-α-D-galacto-2-nonulopyranoylonate)-D-glycero-α-D-galacto-2-nonulopyranoylonate)-4-O-(2,2,2-trichloroethoxycarbonyl)-1-thio-α-D-galactopyranoside (23) (3.86 g, 2.46 mmol, 88%, α/β=>95/5). The α/β ratio was determined by ¹H NMR analysis. ¹H NMR (600 MHz, CDCl₃) δ 8.05 (d, 2H, J=7.7 Hz), 7.59 (t, 1H, J=7.4 Hz), 7.47 (t, 2H, J=7.7 Hz), 7.22-7.37 (m, 15H), 7.10 (d, 2H, J=5.9 Hz), 7.04 (d, 2H, J=8.0 Hz), 5.75 (br-s, 1H), 5.38 (dd, 1H, J=10.2, 1.2 Hz), 5.31 (dt, 1H, J=9.9, 2.2 Hz), 5.29 (br-s, 1H), 5.13 (d, 1H, J=3.1 Hz), 4.79 (d, 1H, J=11.8 Hz), 4.66 (d, 1H, J=11.8 Hz), 4.59 (d, 1H, J=12.1 Hz), 4.47 (d, 1H, J=11.8 Hz), 4.45 (d, 1H, J=11.8 Hz), 4.33-4.37 (m, 3H), 4.17-4.23 (m, 3H), 3.93 (d, 1H, J=14.9 Hz), 3.80-3.85 (m, 3H), 3.75 (s, 3H), 3.72 (s, 3H), 3.66-3.74 (m, 5H), 3.49-3.58 (m, 4H), 3.38 (d, 1H, J=8.7 Hz), 3.17 (br-s, 1H), 2.99 (t, 1H, J=10.4 Hz), 2.77 (dd, 1H, J=12.2, 3.3 Hz), 2.69 (br-s, 1H), 2.65 (dd, 1H, J=11.7, 3.0 Hz), 2.28 (s, 3H), 2.04 (t, 1H, J=12.8 Hz), 1.81 (t, 1H, J=12.5 Hz); ¹³C NMR (150 MHz, CDCl₃) δ 168.4, 167.9, 167.7, 167.2, 159.3, 159.1, 154.1, 138.5, 137.7, 137.1, 137.0, 133.7, 133.3, 130.0, 129.9, 128.84, 128.79, 128.7, 128.6, 128.44, 128.42, 128.30, 128.25, 128.2, 128.09, 128.06, 127.9, 101.3, 99.0, 94.6, 78.2, 77.8, 76.6, 76.0, 74.5, 74.4, 74.2, 74.0, 73.9, 73.6, 73.5, 72.6, 70.3, 69.4, 68.0, 67.1, 58.9, 57.5, 53.7, 53.5, 41.5, 40.4, 37.2, 35.8; HRMS (ESI-TOF) Calcd for C₇₀H₇₃N₂O₂₆SCl₅Na [M+Na]⁺ 1587.2513, found 1587.2524.

### 5-chloropentyl 4-O-(2-O-benzoyl-6-O-benzyl-3-O-(methyl 5-amino-9-O-benzyl-5-N,4-O-carbonyl-3,5-dideoxy-8-O-(methyl 5-amino-9-O-benzyl-5-N4-O-carbonyl-7,8-di-O-chloroacetyl-3,5-dideoxy-D-glycero-α-D-galacto-2-nonulopyranosylonate)-D-glycero-α-D-galacto-2-nonulopyranoylonate)-4-O-(2,2,2-trichloroethoxycarbonyl)-β-D-galactopyranoside)-2,3,6-tri-O-benzyl-β-D-glucopyranoside (8)

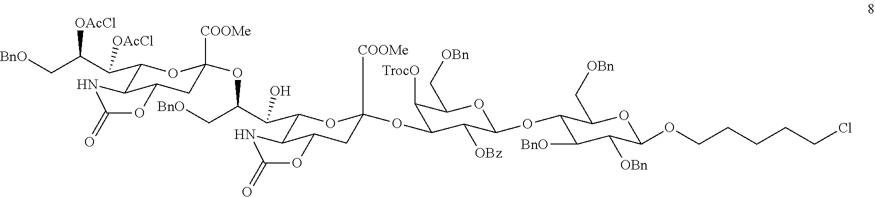

To a stirred solution of 4-methylphenyl 2-O-benzoyl-6-O-benzyl-3-O-(methyl 5-amino-9-O-benzyl-5-N4-O-carbonyl-3,5-dideoxy-8-O-(methyl 5-amino-9-O-benzyl-5-N4-O-carbonyl-7,8-di-O-chloroacetyl-3,5-dideoxy-D-glycero-α-D-galacto-2-nonulopyranoylonate)-D-glycero-α-D-galacto-2-nonulopyranoylonate)-4-O-(2,2,2-trichloroethoxycarbonyl)-1-thio-α-D-galactopyranoside (23) (1.94 g, 1.24 mmol, 1.00 eq.), 5-chloropentyl-2,3,6-tri-O-benzyl-β-D-glucopyranoside (11) (1.37 g, 2.47 mmol, 2.00 eq.) and pulverized activated MS-4 Å in a mixture of dry CH₂Cl₂ (21 mL) and dry acetonitrile (14 mL) was added N-iodosuccinimide (0.61 g, 2.71 mmol, 2.20 eq.) and 0.5 M trifluoromethanesulfonic acid solution in dry Et₂O (0.50 mL, 0.25 mmol, 0.20 eq.) at room temperature under argon. After being stirred at the same temperature for 30 min, the reaction mixture was diluted with CH₂Cl₂ and filtered through a pad of celite. The filtrate was poured into a mixture of saturated aq. NaHCO₃ and saturated aq. Na₂S₂O₃. The aqueous layer was extracted with two portions of CH₂Cl₂. The combined extracts were washed with saturated aq. NaHCO₃ and saturated aq. Na₂S₂O₃ and brine, dried over MgSO₄, filtered, and evaporated in vacuo. The residue was chromatographed on silica gel with 50:50 hexane-ethyl acetate to give 5-chloropentyl 4-O-(2-O-benzoyl-6-O-benzyl-3-O-(methyl 5-amino-9-O-benzyl-5-N4-O-carbonyl-3,5-dideoxy-8-O-(methyl 5-amino-9-O-benzyl-5-N4-O-carbonyl-7, 8-di-O-chloroacetyl-3,5-dideoxy-D-glycero-α-D-galacto-2-nonulopyranosylonate)-D-glycero-α-D-galacto-2-nonulopyranoylonate)-4-O-(2,2,2-trichloroethoxycarbonyl)-β-D-galactopyranoside)-2,3,6-tri-O-benzyl-β-D-glucopyranoside (8) (1.99 g, 1.00 mmol, 81%). ¹H NMR (600 MHz, CDCl₃) δ 7.95 (d, 2H, J=7.6 Hz), 7.58 (t, 1H, J=7.4 Hz), 7.45 (t, 2H, J=7.7 Hz), 7.19-7.37 (m, 28H), 7.12 (d, 2H, J=6.8 Hz), 5.94 (br-s, 1H), 5.39 (dd, 1H, J=10.2, 1.3 Hz), 5.35 (dt, 1H, J=10.1, 2.6 Hz), 5.30 (br-s, 1H), 5.11 (d, 1H, J=3.3 Hz), 4.91 (d, 1H, J=10.8 Hz), 4.86 (d, 1H, J=11.8 Hz), 4.85 (d, 1H, J=11.0 Hz), 4.79 (d, 1H, J=10.8 Hz), 4.70 (d, 1H, J=11.0 Hz), 4.66 (d, 1H, J=11.9 Hz), 4.60 (d, 2H, J=12.1 Hz), 4.17-4.38 (m, 11H), 3.94 (d, 1H, J=14.9 Hz), 3.89 (t, 1H, J=9.4 Hz), 3.78-3.86 (m, 4H), 3.76 (s, 3H), 3.69-3.73 (m, 2H), 3.68 (s, 3H), 3.63 (t, 1H, J=8.5 Hz), 3.50-3.55 (m, 6H), 3.44 (t, 2H, J=6.7 Hz), 3.30-3.43 (m, 6H), 3.26 (t, 2H, J=8.9 Hz), 3.19 (dt, 1H, J=9.5, 2.7 Hz), 3.00 (t, 1H, J=10.7 Hz), 2.79 (dd, 1H, J=12.1, 3.3 Hz), 2.72 (t, 1H, J=9.8 Hz), 2.50 (dd, 1H, J=12.1, 3.3 Hz), 2.05 (t, 1H, J=12.8 Hz), 1.83 (t, 1H, J=12.4 Hz), 1.69-1.75 (m, 2H), 1.41-1.63 (m, 4H); ¹³C NMR (150 MHz, CDCl₃) δ 168.4, 167.9, 167.3, 167.2, 159.2, 159.1, 154.1, 139.2, 138.7, 138.5, 137.8, 137.1, 136.9, 133.8, 129.8, 129.6, 128.9, 128.81, 128.78, 128.61, 128.58, 128.49, 128.46, 128.4, 128.1, 128.06, 128.03, 127.9, 127.85, 127.76, 127.4, 103.6, 101.3, 100.2, 99.5, 94.6, 82.8, 82.0, 78.5, 78.0, 77.3, 77.1, 76.7, 76.6, 75.6, 75.0, 74.8, 74.5, 74.3, 74.2, 74.0, 73.7, 73.6, 72.8, 71.6, 70.3, 69.8, 69.3, 68.1, 67.1, 59.2, 57.5, 53.7, 53.4, 45.0, 41.5, 40.4, 37.3, 35.0, 32.5, 29.1, 23.7; HRMS (ESI-TOF) Calcd for C₉₅H₁₀₄N₂O₃₂Cl₆Na [M+Na]⁻ 2017.4601, found 2017.4623.

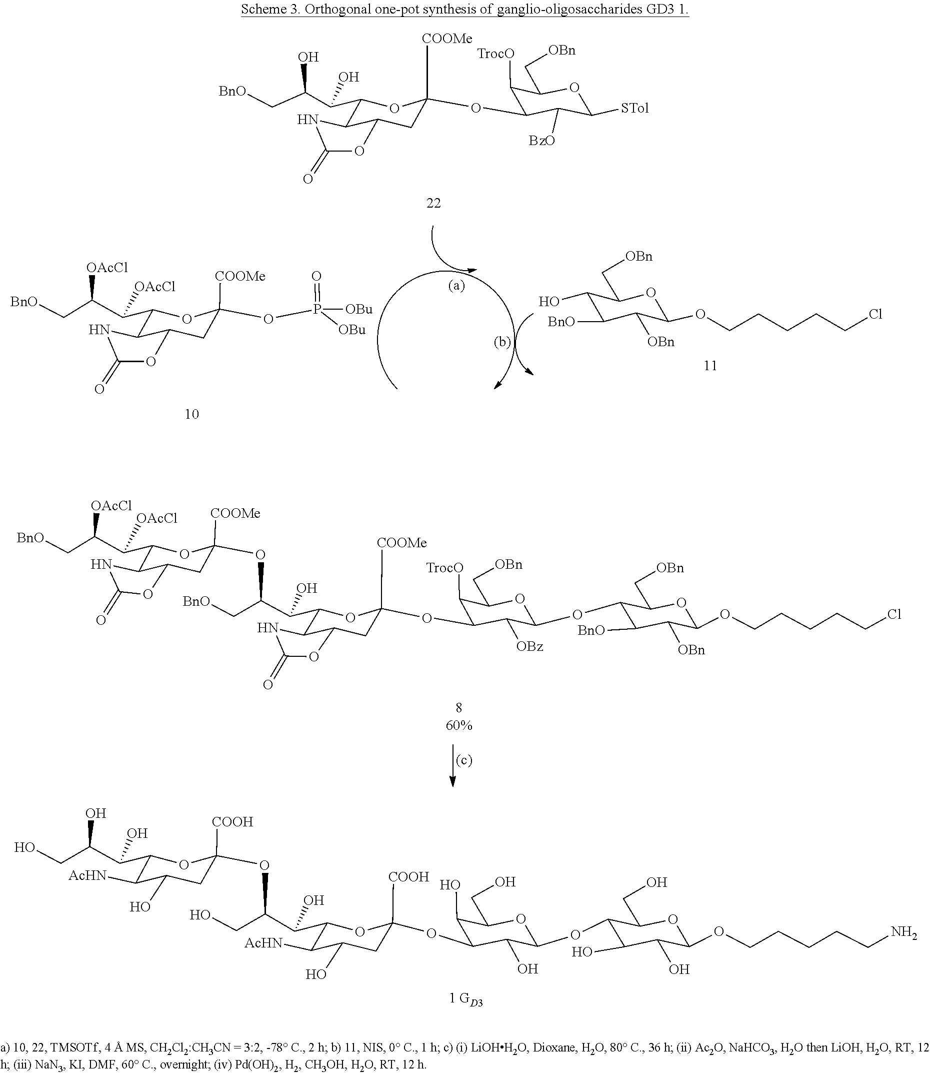

### 5-aminopentyl 4-O-(5-acetoamide-3,5-dideoxy-8-O-(5-acetoamide-3,5-di deoxy-D-glycero-α-D-galacto-2-nonulopyranosylonate)-D-glycero-α-D-galacto-2-nonulopyranosylonate)-β-D-galactopyranoside)-β-D-glucopyranoside (1) (No. 19) (G19)

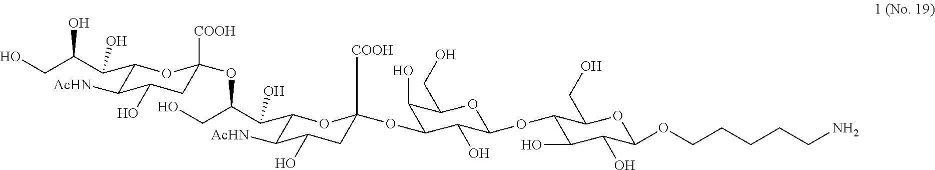 [SMILES: CC(=O)NC1C(O)CC(O[C@H](CO)[C@@H](O)C2OC(OC3C(O)C(CO)OC(OC4C(CO)OC(OCCCCCN)C(O)C4O)C3O)(C(=O)O)CC(O)C2NC(C)=O)(C(=O)O)OC1[C@H](O)[C@H](O)CO]

To a stirred solution of 5-azidopentyl 4-O-(2-O-benzoyl-6-O-benzyl-3-O-(methyl 5-amino-9-O-benzyl-5-N4-O-carbonyl-3,5-dideoxy-8-O-(methyl 5-amino-9-O-benzyl-5-N4-O-carbonyl-7,8-di-O-chloroacetyl-3,5-dideoxy-D-glycero-α-D-galacto-2-nonulopyranosylonate)-D-glycero-α-D-galacto-2-nonulopyranoylonate)-4-O-(2,2,2-trichloroethoxycarbonyl)-β-D-galactopyranoside)-2,3,6-tri-O-benzyl-β-D-glucopyranoside (8) (300 mg, 0.15 mmol, 1.00 eq.) in a mixture of 15 mL 1,4-dioxane and 15 mL H₂O was added LiOH (179 mg) at room temperature. After being stirred at 80° C. for 30 h, the reaction mixture was evaporated in vacuo. The residue was purified by reverse-phase column chromatography (LiChroprep® RP-18). To a stirred solution of above residue in 45 mL H₂O was added NaHCO₃ (1.50 g) and acetic anhydride (750 μL) at room temperature. After being stirred at the same temperature for 1 h, the reaction mixture was added NaHCO₃ (1.50 g) and acetic anhydride (750 μL). After being stirred at the same temperature for another 1 h, the reaction mixture was added LiOH (1.50 g) at room temperature. After being stirred at the same temperature for 12 h, the reaction mixture was evaporated in vacuo. The residue was purified by reverse-phase column chromatography (LiChroprep® RP-18). To a stirred solution of above residue in 10 mL dry DMF was added NaN₃ (37 mg, 0.57 mmol, 5.00 eq) and KI (2 mg, 0.01 mmol, 0.10 eq.) at room temperature. After being stirred at 60° C. overnight, the reaction mixture was evaporated in vacuo. The residue was purified by reverse-phase column chromatography (LiChroprep® RP-18). To a stirred solution of above residue in a mixture of 15 mL methanol and 15 mL H₂O was added Pd(OH)₂ (750 mg) at room temperature. After being stirred at room temperature under H₂ gas atmosphere overnight, the reaction mixture was filtered through a pad of celite and the filtrate was evaporated in vaccuo. The residue was purified by reverse-phase column chromatography (LiChroprep® RP-18) to give 5-aminopentyl 4-O-(3-O-(5-acetoamide-3,5-dideoxy-8-O-(5-acetoamide-3,5-dideoxy-D-glycero-α-D-galacto-2-nonulopyranosylonate)-D-glycero-α-D-galacto-2-nonulopyranosylonate)-β-D-galactopyranoside)-β-D-glucopyranoside (1) (92 mg, 0.09 mmol, 60%). ¹H NMR (600 MHz, CDCl₃) δ 4.51 (d, 1H, J=7.9 Hz), 4.48 (d, 1H, J=8.0 Hz), 4.18 (dd, 1H, J=12.2, 3.6 Hz), 4.13 (m, 1H), 4.08 (dd, 1H, J=9.9, 3.1 Hz), 4.00 (dd, 1H, J=12.2, 2.0 Hz), 3.95 (d, 1H, J=3.1 Hz), 3.79-3.93 (m, 7H), 3.54-3.76 (m, 15H), 3.30 (dd, 1H, J=9.2, 8.2 Hz), 3.00 (t, 2H, J=7.5 Hz), 2.77 (dd, 1H, J=12.3, 4.6 Hz), 2.67 (dd, 1H, J=12.3, 4.4 Hz), 2.06 (s, 3H), 2.02 (s, 3H), 1.64-1.75 (m, 6H), 1.43-1.48 (m, 2H); ¹³C NMR (150 MHz, CDCl₃) δ 177.7, 176.2, 176.1, 105.4, 104.8, 103.3, 102.9, 80.9, 80.7, 78.2, 78.0, 77.5, 77.1, 76.7, 75.6, 75.4, 74.5, 72.8, 72.0, 71.9, 71.2, 70.8, 70.6, 70.2, 65.3, 64.3, 63.8, 62.7, 55.0, 54.5, 43.2, 42.4, 42.1, 30.9, 29.1, 25.0, 24.8; HRMS (ESI-TOF) Calcd for C₃₉H₆₆N₃O₂₇ [M−H]⁻ 1008.3884, found 1008.3887.

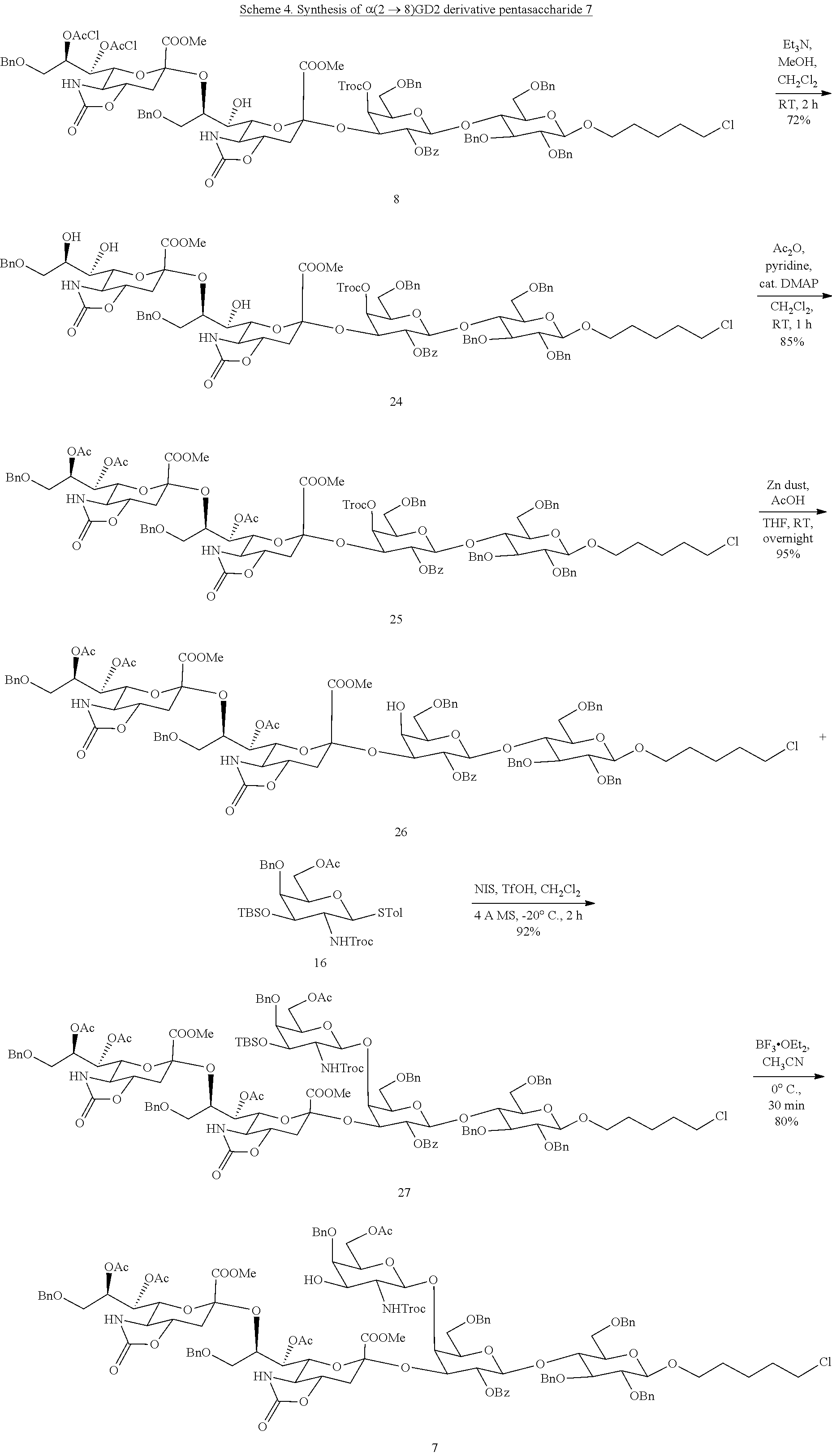

### 5-chloropentyl 4-O-(2-O-benzoyl-6-O-benzyl-3-O-(methyl 5-amino-9-O-benzyl-5-N,4-O-carbonyl-3,5-dideoxy-8-O-(methyl 5-amino-9-O-benzyl-5-N4-O-carbonyl-3,5-dideoxy-D-glycero-α-D-galacto-2-nonulopyranosylonate)-D-glycero-α-D-galacto-2-nonulopyranoylonate)-4-O-(2,2,2-trichloroethoxycarbonyl)-β-D-galactopyranoside)-2,3,6-tri-O-benzyl-β-D-glucopyranoside (24)

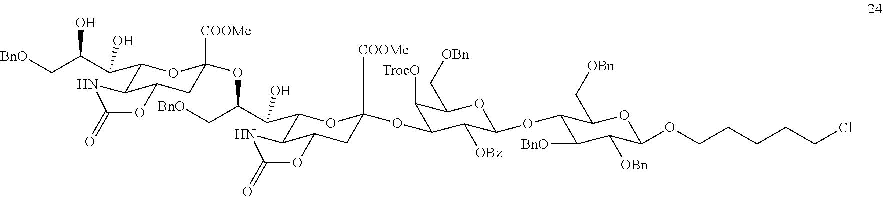

To a stirred solution of 5-chloropentyl 4-O-(2-O-benzoyl-6-O-benzyl-3-O-(methyl 5-amino-9-O-benzyl-5-N4-O-carbonyl-3,5-dideoxy-8-O-(methyl 5-amino-9-O-benzyl-5-N4-O-carbonyl-7,8-di-O-chloroacetyl-3,5-dideoxy-D-glycero-α-D-galacto-2-nonulopyranosylonate)-D-glycero-α-D-galacto-2-nonulopyranoylonate)-4-O-(2,2,2-trichloroethoxycarbonyl)-β-D-galactopyranoside)-2,3,6-tri-O-benzyl-β-D-glucopyranoside (8) (2.40 g, 1.20 mmol, 1.00 eq.) in a mixture of 10 mL dry CH₂Cl₂ and 15 mL methanol was added Et₃N (0.47 mL, 3.62 mmol, 3.00 eq.) at room temperature. After being stirred at the same temperature for 2 h, the reaction mixture was neutralized with Amberlite IR-120 resin and filtered. The filtrate was evaporated in vaccuo. The residue was chromatographed on silica gel with 40:60 hexane-ethyl acetate to give 5-chloropentyl 4-O-(2-O-benzoyl-6-O-benzyl-3-O-(methyl 5-amino-9-O-benzyl-5-N4-O-carbonyl-3,5-dideoxy-8-O-(methyl 5-amino-9-O-benzyl-5-N4-O-carbonyl-3,5-dideoxy-D-glycero-α-D-galacto-2-nonulopyranosylonate)-D-glycero-α-D-galacto-2-nonulopyranoylonate)-4-O-(2,2,2-trichloroethoxycarbonyl)-β-D-galactopyranoside)-2,3,6-tri-O-benzyl-β-D-glucopyranoside (24) (1.59 g, 0.86 mmol, 72%). ¹H NMR (600 MHz, CDCl₃) δ 7.95 (d, 2H, J=7.6 Hz), 7.58 (t, 1H, J=7.4 Hz), 7.42 (t, 2H, J=7.8 Hz), 7.36 (d, 2H, J=7.4 Hz), 7.23-7.32 (m, 22H), 7.17-7.21 (m, 6H), 6.21 (br-s, 1H), 5.76 (br-s, 1H), 5.16 (d, 1H, J=3.1 Hz), 4.92 (d, 1H, J=10.8 Hz), 4.88 (d, 1H, J=11.8 Hz), 4.85 (d, 2H, J=11.2 Hz), 4.79 (d, 1H, J=10.7 Hz), 4.71 (d, 1H, J=11.1 Hz), 4.64 (d, 1H, J=11.9 Hz), 4.57 (d, 1H, J=12.2 Hz), 4.52 (d, 1H, J=12.6 Hz), 4.50 (d, 1H, J=12.8 Hz), 4.42 (d, 1H, J=11.6 Hz), 4.38 (d, 1H, J=11.6 Hz), 4.31 (d, 1H, J=12.2 Hz), 4.25 (d, 1H, J=11.6 Hz), 4.24 (d, 1H, J=7.7 Hz), 4.18 (d, 1H, J=11.6 Hz), 4.15 (br-s, 1H), 3.92-3.97 (m, 2H), 3.89 (t, 1H, J=9.3 Hz), 3.81-3.86 (m, 2H), 3.75-3.77 (m, 1H), 3.67 (s, 3H), 3.62 (s, 3H), 3.62-3.68 (m, 4H), 3.47-3.57 (m, 9H), 3.44 (t, 2H, J=6.7 Hz), 3.41-3.44 (m, 1H), 3.35 (dd, 2H, J=8.0, 9.1 Hz), 3.24-3.32 (m, 2H), 3.19 (dt, 1H, J=10.0, 2.9 Hz), 2.90 (dd, 2H, J=11.7, 3.2 Hz), 2.50 (d, 1H, J=9.3 Hz), 2.08 (t, 1H, J=12.5 Hz), 1.95 (t, 1H, J=12.3 Hz), 1.71-1.75 (m, 2H), 1.56-1.64 (m, 2H), 1.42-1.53 (m, 2H); ¹³C NMR (150 MHz, CDCl₃) 8168.8, 167.2, 160.2, 160.0, 154.2, 139.2, 138.7, 138.5, 137.9, 137.8, 137.2, 133.9, 129.8, 129.5, 128.9, 128.8, 128.7, 128.60, 128.57, 128.5, 128.4, 128.1, 128.0, 127.9, 127.82, 127.78, 127.42, 103.7, 99.9, 99.8, 94.6, 82.7, 82.0, 77.1, 76.8, 76.4, 75.7, 75.1, 74.8, 74.6, 74.5, 74.1, 73.9, 73.8, 73.7, 73.5, 72.3, 71.7, 71.6, 71.2, 71.0, 69.8, 68.2, 67.0, 59.7, 57.4, 53.7, 53.5, 45.1, 35.8, 32.5, 29.1, 23.7; HRMS (ESI-TOF) Calcd for C₉₁H₁₀₂N₂O₃₀Cl₄Na [M+Na]⁺ 1865.5169, found 1865.5176.

### 5-chloropentyl 4-O-(2-O-benzoyl-6-O-benzyl-3-O-(methyl 7-O-acetyl-5-amino-9-O-benzyl-5-N4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-4-O-(2,2,2-trichloroethoxycarbonyl)-β-D-galactopyranoside)-2,3,6-tri-O-benzyl-β-D-glucopyranoside (25)

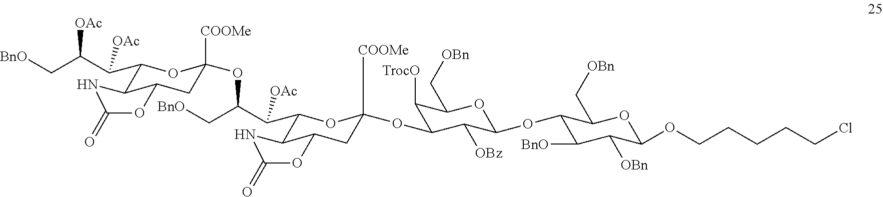

To a stirred solution of 5-chloropentyl 4-O-(2-O-benzoyl-6-O-benzyl-3-O-(methyl 5-amino-9-O-benzyl-5-N4-O-carbonyl-3,5-dideoxy-8-O-(methyl 5-amino-9-O-benzyl-5-N4-O-carbonyl-3,5-dideoxy-D-glycero-α-D-galacto-2-nonulopyranosylonate)-D-glycero-α-D-galacto-2-nonulopyranoylonate)-4-O-(2,2,2-trichloroethoxycarbonyl)-β-D-galactopyranoside)-2,3,6-tri-O-benzyl-β-D-glucopyranoside (24) (970 mg, 0.53 mmol, 1.00 eq.) in 25 mL dry CH₂Cl₂ was added acetic anhydride (0.45 mL, 4.73 mmol, 9.00 eq.), pyridine (0.51 mL, 6.31 mmol, 12.00 eq.) and DMAP (6 mg, 0.05, 0.10 eq.) at room temperature. After being stirred at the same temperature for 1 h, the reaction mixture was poured into 1M aq. HCl. The aqueous phase was washed with two portions of CH₂Cl₂. The combined extracts were washed with saturated aq. NaHCO₃ and brine, dried over MgSO₄, filtered, and evaporated in vacuo. The residue was chromatographed on silica gel with 57:43 hexane-ethyl acetate to give 5-chloropentyl 4-O-(2-O-benzoyl-6-O-benzyl-3-O-(methyl 7-O-acetyl-5-amino-9-O-benzyl-5-N4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-4-O-(2,2,2-trichloroethoxycarbonyl)-β-D-galactopyranoside)-2,3,6-tri-O-benzyl-β-D-glucopyranoside (25) (893 mg, 0.45 mmol, 85%). ¹H NMR (600 MHz, CDCl₃) δ 8.12 (d, 2H, J=7.3 Hz), 7.54 (t, 1H, J=7.4 Hz), 7.47 (t, 2H, J=7.7 Hz), 7.17-7.35 (m, 28H), 6.99 (d, 1H, J=5.5 Hz), 6.98 (d, 1H, J=7.7 Hz), 5.75 (br-s, 1H), 5.34-5.37 (m, 2H), 5.30 (d, 1H, J=11.2 Hz), 5.28 (d, 1H, J=11.3 Hz), 5.10 (d, 1H, J=3.1 Hz), 4.94 (d, 1H, J=10.9 Hz), 4.91 (dd, 1H, J=6.1, 2.0 Hz), 4.85 (d, 1H, J=11.9 Hz), 4.84 (d, 1H, J=11.2 Hz), 4.83 (d, 1H, J=8.5 Hz), 4.79 (d, 1H, J=10.7 Hz), 4.70 (d, 1H, J=11.1 Hz), 4.64 (d, 1H, J=11.9 Hz), 4.59 (d, 1H, J=12.1 Hz), 4.54 (d, 1H, J=12.1 Hz), 4.42 (d, 1H, J=11.2 Hz), 4.20-4.36 (m, 7H), 4.08 (d, 1H, J=10.0 Hz), 4.05 (dd, 1H, J=5.6, 3.1 Hz), 3.90 (t, 1H, J=9.3 Hz), 3.74 (s, 3H), 3.72-3.86 (m, 5H), 3.71 (s, 3H), 3.43 (t, 2H, J=6.7 Hz), 3.40-3.60 (m, 8H), 3.29-3.36 (m, 3H), 3.18-3.20 (m, 1H), 2.97 (t, 1H, J=10.6 Hz), 2.86 (dd, 1H, J=12.1, 3.4 Hz), 2.61 (t, 1H, J=10.4 Hz), 2.58 (dd, 1H, J=11.9, 3.5 Hz), 2.16 (s, 3H), 2.01 (t, 1H, J=12.8 Hz), 1.88 (s, 3H), 1.76 (s, 3H), 1.70-1.75 (m, 3H), 1.55-1.63 (m, 2H), 1.40-1.52 (m, 2H); ¹³C NMR (150 MHz, CDCl₃) δ 171.3, 170.44, 170.38, 168.0, 167.9, 168.4, 159.4, 159.1, 154.0, 139.3, 138.77, 138.76, 137.9, 137.4, 136.9, 133.6, 130.2, 129.8, 129.0, 128.75, 128.69, 128.53, 128.51, 128.48, 128.4, 128.34, 128.26, 128.2, 128.03, 127.97, 127.8, 127.7, 127.5, 127.4, 103.6, 100.9, 100.7, 98.5, 94.8, 82.9, 82.1, 76.9, 76.7, 76.12, 76.06, 75.6, 75.0, 74.8, 74.2, 73.9, 73.8, 73.6, 73.3, 72.1, 71.2, 71.7, 71.3, 69.7, 68.9, 68.3, 67.9, 67.6, 67.4, 59.0, 57.9, 53.4, 53.3, 45.1, 37.4, 36.2, 32.5, 29.2, 23.7, 21.6, 20.8, 20.7; HRMS (ESI-TOF) Calcd for C₉₇H₁₀₈N₂O₃₃Cl₄Na [M+Na]⁺ 1991.5486, found 1991.5492.

### 5-chloropentyl 4-O-(2-O-benzoyl-6-O-benzyl-3-O-(methyl 7-O-acetyl-5-amino-9-O-benzyl-5-N4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O-benzyl-β-D-glucopyranoside (26)

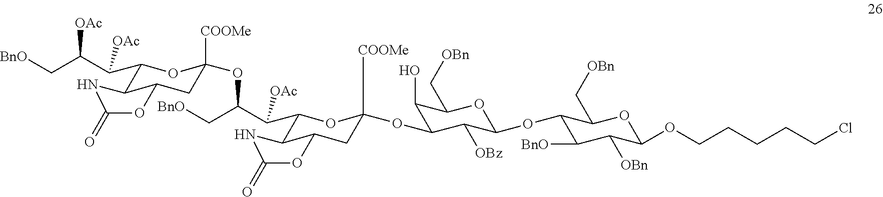

To a stirred solution of 5-chloropentyl 4-O-(2-O-benzoyl-6-O)-benzyl-3-O-(methyl 7-O-acetyl-5-amino-9-O-benzyl-5-N4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-4-O-(2,2,2-trichloroethoxycarbonyl)-β-D-galactopyranoside)-2,3,6-tri-O-benzyl-β-D-glucopyranoside (25) (865 mg, 0.44 mmol, 1.00 eq.) in a mixture of 20 mL dry THF and 5 mL acetic acid was added activated Zn-dust (1.43 g, 21.87 mmol, 50.00 eq.) at room temperature. After being stirred at the same temperature for 1 h, the reaction mixture was diluted with ethyl acetate and filtered through a pad of celite. The filtrate mixture was poured into a mixture of saturated aq. NaHCO₃. The aqueous layer was extracted with two portions of ethyl acetate. The combined extracts were washed with brine, dried over MgSO₄, filtered, and evaporated in vacuo. The residue was chromatographed on silica gel with 45:55 hexane-ethyl acetate to give 5-chloropentyl 4-O-(2-O-benzoyl-6-O-benzyl-3-O-(methyl 7-O-acetyl-5-amino-9-O-benzyl-5-N4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulo-pyranosylonate)-D-glycero-α-D-galacto-2-nonulopyranoylonate)-β-D-galactopyranoside)-2,3,6-tri-O-benzyl-β-D-glucopyranoside (26) (763 mg, 0.42 mmol, 95%). ¹H NMR (600 MHz, CDCl₃) δ 7.97 (d, 2H, J=7.7 Hz), 7.56 (t, 1H, J=7.4 Hz), 7.43 (t, 2H, J=7.7 Hz), 7.20-7.34 (m, 28H), 7.14-7.15 (m, 2H), 5.65 (br-s, 1H), 5.39 (br-s, 1H), 5.36 (t, 1H, J=8.9 Hz), 5.32 (d, 1H, J=10.8 Hz), 5.30 (d, 1H, J=10.3 Hz), 5.01 (t, 1H, J=4.1 Hz), 4.94 (d, 1H, J=10.8 Hz), 4.82 (d, 1H, J=11.0 Hz), 4.78 (d, 2H, J=9.3 Hz), 4.66 (d, 1H, J=11.0 Hz), 4.59 (d, 1H, J=12.2 Hz), 4.53 (d, 1H, J=12.1 Hz), 4.52 (d, 1H, J=11.6 Hz), 4.37 (d, 1H, J=11.9 Hz), 4.29-4.33 (m, 4H), 4.23 (d, 1H, J=7.8 Hz), 4.21 (dd, 1H, J=9.9, 3.5 Hz), 4.07 (d, 1H, J=9.9 Hz), 3.96 (dd, 1H, J=7.7, 3.6 Hz), 3.80-3.90 (m, 7H), 3.76 (s, 3H), 3.64 (dd, 1H, J=8.7, 7.2 Hz), 3.55 (s, 3H), 3.52-3.56 (m, 4H), 3.38-3.48 (m, 8H), 3.34 (t, 2H, J=8.5 Hz), 3.22 (dt, 1H, J=9.7, 2.0 Hz), 3.01 (t, 1H, J=10.5 Hz), 2.97 (dd, 1H, J=12.1, 3.2 Hz), 2.75 (t, 1H, J=10.4 Hz), 2.52 (br-s, 1H), 2.43 (dd, 1H, J=12.0, 3.5 Hz), 2.17 (s, 3H), 2.09 (t, 1H, J=12.8 Hz), 1.96 (t, 1H, J=12.5 Hz), 1.87 (s, 3H), 1.70-1.74 (m, 2H), 1.64 (s, 3H), 1.55-1.61 (m, 2H), 1.41-1.52 (m, 2H); ¹³C NMR (150 MHz, CDCl₃) δ 171.3, 170.8, 170.5, 168.1, 167.7, 164.9, 159.5, 159.2, 139.3, 138.74, 138.67, 138.2, 137.5, 137.4, 133.6, 129.9, 129.8, 128.9, 128.74, 128.67, 128.6, 128.5, 128.4, 128.31, 128.29, 128.25, 128.18, 128.1, 128.0, 127.9, 127.8, 127.7, 127.4, 103.6, 101.5, 100.7, 99.6, 83.0, 82.1, 76.9, 76.4, 75.49, 75.46, 75.4, 75.0, 74.9, 74.7, 74.1, 73.7, 73.64, 73.59, 73.4, 73.2, 72.6, 71.1, 69.7, 69.4, 68.9, 68.4, 68.23, 68.15, 67.8, 67.5, 58.8, 57.9, 53.5, 53.2, 45.1, 37.5, 35.1, 32.5, 29.2, 23.7, 21.5, 20.8, 20.5; HRMS (ESI-TOF) Calcd for C₉₄H₁₀₇N₂₃₁ClNa [M+Na]⁺ 1817.6444, found 1817.6444.

### 5-chloropentyl 4-O-(4-O-(6-O-acetyl-4-O-benzyl-3-O-tert-butyldimethyl-silyl-2-deoxy-2-(2,2,2-trichloroethyoxycarbonylamino)-β-D-galactopy-ranoside)-2-O-benzoyl-6-O-benzyl-3-O-(methyl 7-O-acetyl-5-amino-9-O-benzyl-5-N4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O-benzyl-β-D-glucopyranoside (27)

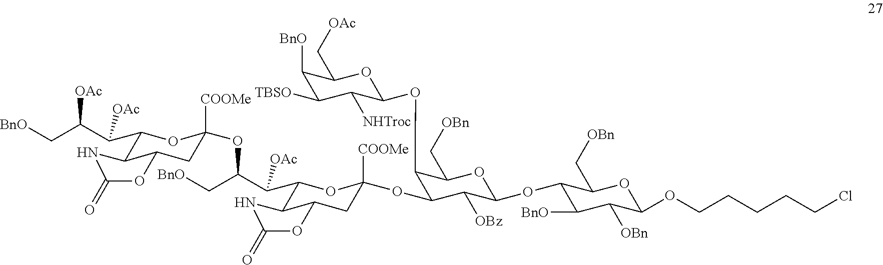

To a stirred solution of 5-chloropentyl 4-O-(2-O-benzoyl-6-O-benzyl-3-O-(methyl 7-O-acetyl-5-amino-9-O-benzyl-5-N4-O-carbonyl-3,5-dideoxy-8-O-(methyl 5-amino-9-O)-benzyl-5-N4-O-carbonyl-7,8-O)-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O)-benzyl-β-D-glucopyranoside (26) (688 mg, 0.38 mmol, 1.00 eq.), 4-methylphenyl 6-O-acetyl-4-O-benzyl-3-O-tert-butyldimethylsilyl-2-deoxy-1-thio-2-(2,2,2-trichloroethyoxycarbamoyl)-β-D-galactopyranoside (16) (813 mg, 1.15 mmol, 3.00 eq.) and pulverized activated MS-4 Å in dry CH₂Cl₂ (13 mL) was added N-iodosuccinimide (345 mg, 1.53 mmol, 4.00 eq.) and 0.5 M trifluoromethanesulfonic acid solution in dry Et₂O (0.23 mL, 0.12 mmol, 0.30 eq.) at −20° C. under argon. After being stirred at the same temperature for 2 h, the reaction mixture was diluted with CH₂Cl₂ and filtered through a pad of celite. The filtrate was poured into a mixture of saturated aq. NaHCO₃ and saturated aq. Na₂S₂O₃. The aqueous layer was extracted with two portions of CH₂Cl₂. The combined extracts were washed with saturated aq. NaHCO₃ and saturated aq. Na₂S₂O₃ and brine, dried over MgSO₄, filtered, and evaporated in vacuo. The residue was chromatographed on silica gel with 55:45 hexane-ethyl acetate to give 5-chloropentyl 4-O-(4-O-(6-O-acetyl-4-O-benzyl-3-O-tert-butyldimethylsilyl-2-deoxy-2-(2,2,2-trichloroethyoxycarbonylamino)-β-D-galactopyranoside)-2-O-benzoyl-6-O-benzyl-3-O-(methyl 7-O-acetyl-5-amino-9-O-benzyl-5-N4-O-carbonyl-3,5-di-deoxy-8-O-(methyl 5-amino-9-O-benzyl-5-N4-O-carbonyl-7,8-O)-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O)-benzyl-β-D-glucopyranoside (27) (829 mg, 0.35 mmol, 92%), (Ratio of rotamer A:B=88:12). Rotamer A: ¹H NMR (600 MHz, CDCl₃) δ 7.95 (d, 2H, J=7.6 Hz), 7.54 (t, 1H, J=7.4 Hz), 7.44 (t, 2H, J=7.7 Hz), 7.17-7.39 (m, 32H), 7.07 (d, 2H, J=6.0 Hz), 6.76 (t, 1H, J=7.4 Hz), 5.82 (br-s, 1H), 5.32-5.37 (m, 3H), 5.25 (d, 1H, J=10.6 Hz), 5.250 (d, 1H, J=10.6 Hz), 5.248 (d, 1H, J=10.1 Hz), 5.08 (d, 1H, J=11.1 Hz), 4.93 (d, 1H, J=9.7 Hz), 4.88 (d, 1H, J=11.8 Hz), 4.82 (d, 1H, J=6.4 Hz), 4.81 (d, 1H, J=11.3 Hz), 4.72-4.77 (m, 3H), 4.65 (d, 1H, J=7.7 Hz), 4.58 (d, 1H, J=12.1 Hz), 4.57 (d, 1H, J=12.1 Hz), 4.50 (d, 1H, J=11.2 Hz), 4.47 (d, 1H, J=11.4 Hz), 4.36 (d, 1H, J=11.3 Hz), 4.31 (d, 2H, J=12.1 Hz), 4.26 (d, 1H, J=11.8 Hz), 4.19-4.22 (m, 3H), 4.09-4.13 (m, 2H), 4.00 (dd, 1H, J=9.7, 1.6 Hz), 3.93 (d, 1H, J=9.8 Hz), 3.82-3.86 (m, 5H), 3.742 (s, 3H), 3.736 (s, 3H), 3.72-3.80 (m, 4H), 3.62-3.66 (m, 3H), 3.56 (d, 1H, J=1.2 Hz), 3.52 (t, 1H, J=9.0 Hz), 3.47-3.52 (m, 2H), 3.42 (t, 2H, J=6.7 Hz), 3.31-3.45 (m, 5H), 3.27 (dd, 1H, J=9.0, 8.1 Hz), 3.13-3.15 (m, 1H), 2.94 (t, 2H, J=10.4 Hz), 2.88 (dd, 1H, J=11.9, 3.2 Hz), 2.47 (br-d, 1H, J=8.3 Hz), 2.16 (s, 3H), 1.98 (t, 1H, J=12.6 Hz), 1.90 (s, 3H), 1.81 (s, 3H), 1.72 (s, 3H), 1.69-1.74 (m, 3H), 1.53-1.59 (m, 2H), 1.39-1.51 (m, 2H), 0.88 (s, 9H), 0.08 (s, 3H), 0.05 (s, 3H); ¹³C NMR (150 MHz, CDCl₃) δ 171.3, 170.8, 170.7, 170.6, 168.5, 168.3, 164.3, 159.3, 154.2, 139.0, 138.9, 138.79, 138.75, 137.5, 137.3, 133.6, 130.0, 129.5, 128.9, 128.8, 128.6, 128.5, 128.4, 128.3, 128.1, 128.0, 127.9, 127.82, 127.76, 127.7, 127.5, 103.6, 100.5, 100.0, 99.9, 96.1, 82.6, 81.9, 76.6, 76.5, 76.2, 76.0, 75.7, 75.2, 75.1, 74.9, 74.6, 74.2, 74.1, 73.63, 73.56, 73.5, 73.3, 71.9, 71.2, 70.9, 69.7, 69.2, 68.8, 68.3, 67.7, 63.4, 59.2, 57.9, 55.4, 53.7, 53.5, 45.1, 37.5, 35.4, 32.5, 29.2, 26.0, 23.7, 21.7, 20.9, 20.7, 20.6, 18.2, −3.9, −4.9; HRMS (ESI-TOF) Calcd for C₁₁₈H₁₄₁N₃O₃₈Cl₄SiNa [M+Na]⁺ 2398.7614, found 2398.7629.

### 5-chloropentyl 4-O-(4-O-(6-O-acetyl-4-O-benzyl-2-deoxy-2-(2,2,2-trichloroethyoxycarbonylamino)-β-D-galactopyranoside)-2-O-benzoyl-6-O-benzyl-3-O-(methyl 7-O-acetyl-5-amino-9-O-benzyl-5-N4-O-carbonyl-3,5-di-deoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O-benzyl-β-D-glucopyranoside (7)

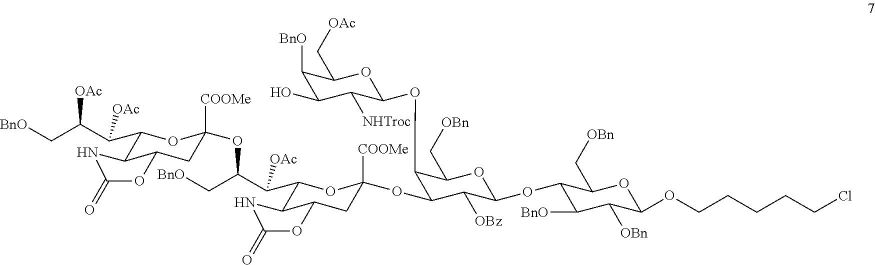

To a stirred solution of 5-chloropentyl 4-O-(4-O-(6-O-acetyl-4-O-benzyl-3-O-tert-butyldimethylsilyl-2-deoxy-2-(2,2,2-trichloroethyoxycarbonylamino)-β-D-galactopyranoside)-2-O-benzoyl-6-O-benzyl-3-O-(methyl 7-O-acetyl-5-amino-9-O-benzyl-5-N4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O-benzyl-β-D-glucopyranoside (27) (1.02 g, 0.44 mmol, 1.00 eq.) in dry acetonitrile (22 mL) was added 48% BF₃·OEt₂ (0.78 mL, 2.64 mmol, 6.00 eq.) at 0° C. under argon. After being stirred at the same temperature for 30 min, the reaction mixture was poured into saturated aq. NaHCO₃. The aqueous phase was washed with two portions of ethyl acetate. The combined extracts were washed with brine, dried over MgSO₄, filtered, and evaporated in vacuo. The residue was chromatographed on silica gel with 50:50 hexane-ethyl acetate to give 5-chloropentyl 4-O-(4-O-(6-O-acetyl-4-O-benzyl-2-deoxy-2-(2,2,2-trichloroethyoxycarbonylamino)-β-D-galactopyranoside)-2-O-benzoyl-6-O-benzyl-3-O-(methyl 7-O-acetyl-5-amino-9-O-benzyl-5-N4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O-benzyl-β-D-glucopyranoside (7) (801 mg, 0.35 mmol, 80%). ¹H NMR (600 MHz, CDCl₃) δ 7.93 (d, 2H, J=7.6 Hz), 7.55 (t, 1H, J=7.4 Hz), 7.44 (t, 2H, J=7.7 Hz), 7.20-7.36 (m, 32H), 7.09-7.10 (m, 2H), 6.94 (t, 1H, J=7.3 Hz), 6.09 (d, 1H, J=5.9 Hz), 5.83 (br-s, 1H), 5.35-5.40 (m, 3H), 5.26 (dd, 1H, J=10.2, 1.5 Hz), 5.06 (d, 1H, J=12.0 Hz), 4.93 (d, 1H, J=11.3 Hz), 4.90 (d, 1H, J=10.1 Hz), 4.827 (d, 1H, J=11.1 Hz), 4.826 (dd, 1H, J=5.2, 2.4 Hz), 4.75 (d, 1H, J=10.0 Hz), 4.72 (d, 2H, J=10.7 Hz), 4.64 (t, 2H, J=11.5 Hz), 4.57 (t, 2H, J=12.0 Hz), 4.56 (d, 1H, J=8.7 Hz), 4.41 (s, 2H), 4.31 (dd, 1H, J=12.1, 3.6 Hz), 4.29 (d, 1H, J=11.3 Hz), 4.21-4.23 (m, 3H), 4.09-4.14 (m, 2H), 4.00 (dd, 1H, J=9.8, 1.4 Hz), 3.78-3.98 (m, 10H), 3.75 (s, 3H), 3.74-3.77 (m, 1H), 3.73 (s, 3H), 3.67 (dd, 1H, J=11.4, 8.3 Hz), 3.49-3.61 (m, 7H), 3.42 (t, 2H, J=6.6 Hz), 3.36-3.45 (m, 5H), 3.30 (t, 1H, J=8.5 Hz), 3.17 (br-d, 1H, J=9.2 Hz), 2.94 (t, 1H, J=10.6 Hz), 2.88 (dd, 1H, J=11.9, 3.2 Hz), 2.84 (t, 1H, J=10.4 Hz), 2.43 (dd, 1H, J=12.2, 3.6 Hz), 2.15 (s, 3H), 2.03 (t, 1H, J=13.0 Hz), 1.97 (t, 1H, J=12.6 Hz), 1.86 (s, 3H), 1.79 (s, 3H), 1.69-1.74 (m, 2H), 1.68 (s, 3H), 1.54-1.59 (m, 2H), 1.40-1.51 (m, 2H); ¹³C NMR (150 MHz, CDCl₃) δ 171.3, 170.8, 170.7, 170.6, 168.3, 167.6, 164.2, 159.29, 159.26, 157.2, 138.8, 138.72, 138.68, 138.3, 137.6, 137.3, 133.7, 129.9, 129.4, 129.0, 128.8, 128.69, 128.67, 128.51, 128.46, 128.38, 128.36, 128.3, 128.2, 128.1, 128.0, 127.8, 127.71, 127.67, 127.6, 127.5, 103.6, 102.2, 100.5, 100.2, 100.0, 95.9, 82.7, 82.0, 76.63, 76.58, 76.2, 76.1, 75.94, 75.87, 75.2, 75.1, 75.0, 74.9, 74.8, 74.1, 73.8, 73.63, 73.56, 73.5, 72.2, 70.9, 69.7, 68.82, 68.79, 68.3, 67.75, 67.69, 63.0, 59.1, 57.9, 56.0, 53.7, 53.5, 45.1, 37.6, 35.2, 32.5, 29.2, 23.7, 21.7, 20.9, 20.6, 20.5; HRMS (ESI-TOF) Calcd for C₁₁₂H₁₂₇N₃O₃₈Cl₄Na [M+Na]⁺ 2284.6749, found 2284.6758.

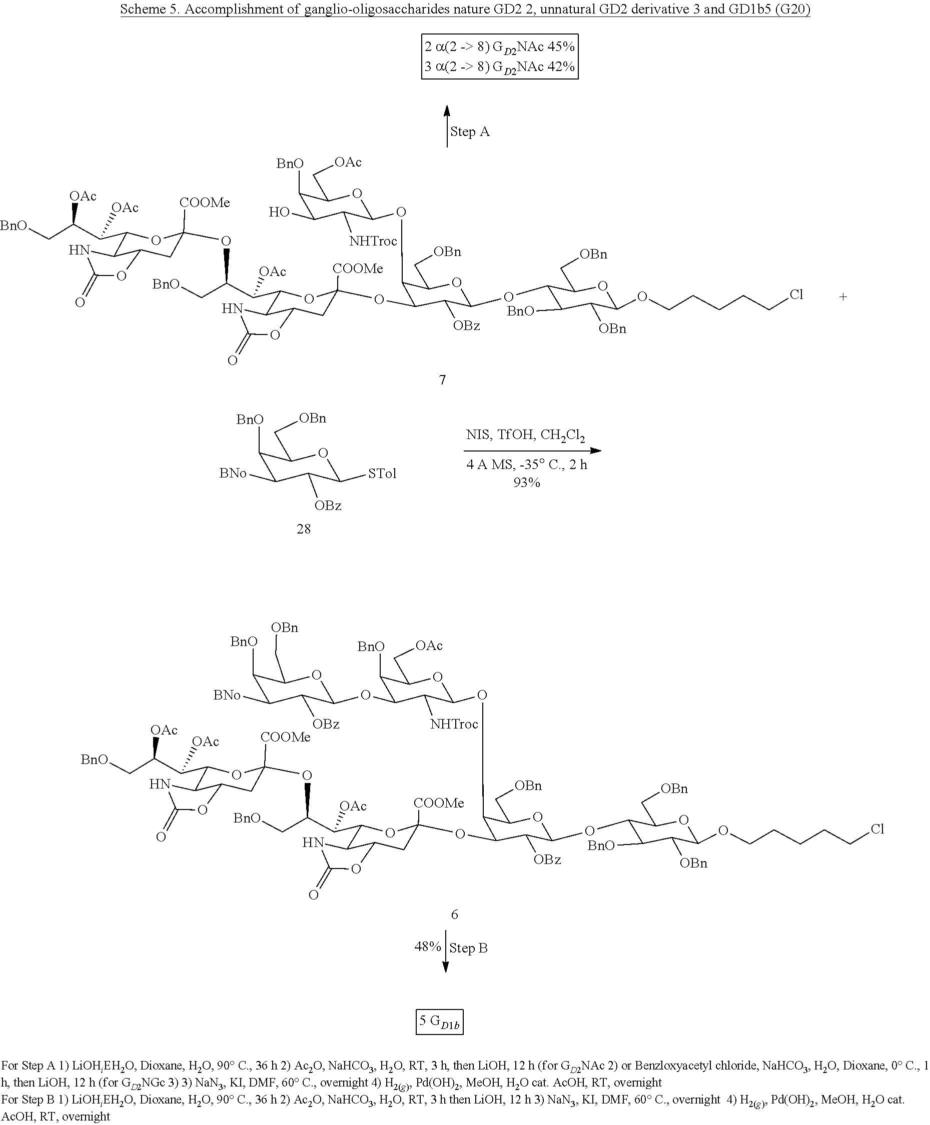

### 5-aminopentyl 4-O-(4-O-(2-acetoamino-2-deoxy-β-D-galactopyranoside)-3-O-(5-acetoamino-3,5-dideoxy-8-O-(5-acetoamino-3,5-dideoxy-D-glycero-α-D-galacto-2-nonulopyranosylonate)-D-glycero-α-D-galacto-2-nonulopyranoylonate)-β-D-galactopyranoside)-α-D-glucopyranoside (2) (No. 18) (G18)

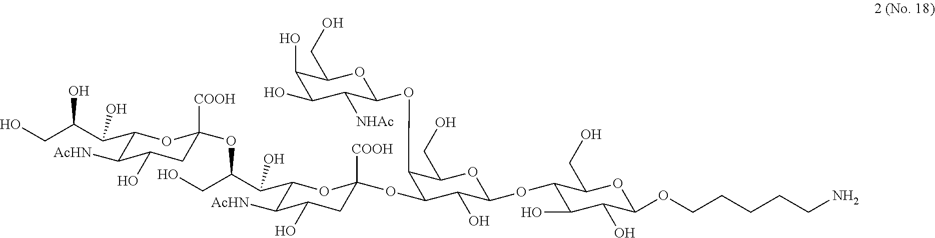 [SMILES: CC(=O)NC1C(OC2C(CO)OC(OC3C(CO)OC(OCCCCCN)C(O)C3O)C(O)C2OC2(C(=O)O)CC(O)C(NC(C)=O)C([C@H](O)[C@@H](CO)OC3(C(=O)O)CC(O)C(NC(C)=O)C([C@H](O)[C@H](O)CO)O3)O2)OC(CO)C(O)C1O]

To a stirred solution of 5-chloropentyl 4-O-(4-O-(6-O-acetyl-4-O-benzyl-2-deoxy-2-(2,2,2-trichloroethyoxycarbonylamino)-β-D-galactopyranoside)-2-O-benzoyl-6-O-benzyl-3-O-(methyl 7-O-acetyl-5-amino-9-O-benzyl-5-N4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O-benzyl-β-D-glucopyranoside (7) (260 mg, 0.11 mmol, 1.00 eq.) in 10 mL THF was added 5 mL 1M aq. NaOH at room temperature. After being stirred at 80° C. overnight, the reaction mixture was evaporated in vacuo. The residue was purified by reverse-phase column chromatography (LiChroprep® RP-18). To a stirred solution of above residue in a mixture of 7.5 mL 1,4-dioxane and 7.5 mL H₂O was added NaHCO₃ (500 mg) and acetic anhydride (250 μL) at room temperature. After being stirred at the same temperature for 1 h, the reaction mixture was added NaHCO₃ (500 mg) and acetic anhydride (250 μL). After being stirred at the same temperature for another 1 h, the reaction mixture was added LiOH (500 mg) at room temperature. After being stirred at the same temperature for 12 h, the reaction mixture was evaporated in vacuo. The residue was purified by reverse-phase column chromatography (LiChroprep® RP-18). To a stirred solution of above residue in 10 mL dry DMF was added NaN₃ (37 mg, 0.57 mmol, 5.00 eq) and KI (2 mg, 0.01 mmol, 0.10 eq.) at room temperature. After being stirred at 60° C. overnight, the reaction mixture was evaporated in vacuo. The residue was purified by reverse-phase column chromatography (LiChroprep® RP-18). To a stirred solution of above residue in a mixture of 7.5 mL methanol and 7.5 mL H₂O was added cat. AcOH and Pd(OH)₂ (375 mg) at room temperature. After being stirred at room temperature under H₂ gas atmosphere overnight, the reaction mixture was filtered through a pad of celite and the filtrate was evaporated in vaccuo. The residue was purified by reverse-phase column chromatography (LiChroprep® RP-18) to give 5-aminopentyl 4-O-(4-O-(2-acetoamino-2-deoxy-β-D-galactopyranoside)-3-O-(5-acetoamino-3,5-dideoxy-8-O)-(5-acetoamino-3,5-dideoxy-D-glycero-α-D-galacto-2-nonulopyranosylonate)-D-glycero-α-D-galacto-2-nonulopyranoylonate)-β-D-galactopyranoside)-β-D-glucopyranoside (2) (58 mg, 0.05 mmol, 45%). ¹H NMR (600 MHz, CDCl₃) δ 4.68 (d, 1H, J=8.4 Hz), 4.48 (d, 1H, J=8.3 Hz), 4.47 (d, 1H, J=8.2 Hz), 4.17 (dd, 1H, J=12.1, 4.0 Hz), 4.14 (dd, 1H, J=10.2, 2.5 Hz), 4.08-4.10 (m, 1H), 4.02 (d, 1H, J=1.8 Hz), 3.98 (d, 1H, J=10.7 Hz), 3.56-3.94 (m, 27H), 3.38 (dd, 1H, J=9.8, 8.1 Hz), 3.28 (t, 1H, J=8.5 Hz), 2.99 (t, 2H, J=7.5 Hz), 2.75 (dd, 1H, J=12.3, 4.5 Hz), 2.67 (dd, 1H, J=12.2, 4.2 Hz), 2.06 (s, 3H), 2.03 (s, 3H), 2.01 (s, 3H), 1.63-1.77 (m, 6H), 1.42-1.47 (m, 2H); ¹³C NMR (150 MHz, CDCl₃) δ 177.71, 177.68, 177.6, 176.1, 176.0, 105.48, 105.45, 104.7, 103.22, 103.20, 81.07, 80.96, 78.5, 77.5, 77.3, 77.2, 77.1, 77.0, 76.4, 75.5, 75.4, 74.5, 73.5, 72.8, 72.4, 72.0, 71.2, 70.8, 70.4, 65.3, 64.2, 63.7, 63.4, 62.7, 55.2, 55.1, 54.5, 43.2, 42.1, 42.0, 30.9, 29.1, 25.3, 25.1, 24.78, 24.75; HRMS (ESI-TOF) Calcd for C₄₇H₇₉N₄O_{32 [M-H]}⁻ 1211.4677, found 1211.4675.

### 2-{[(1S,2R)-1-(6-{[2-({6-[(5-aminopentyl)oxy]-4,5-dihydroxy-2-(hydroxymethyl)oxan-3-yl}oxy)-5-{[4,5-dihydroxy-3-(2-hydroxyacetamido)-6-(hydroxymethyl)oxan-2-yl]oxy}-3-hydroxy-6-(hydroxymethyl)oxan-4-yl]oxy}-6-carboxy-4-hydroxy-3-(2-hydroxyacetamido)oxan-2-yl)-1,3-dihydroxypropan-2-yl]oxy}-4-hydroxy-5-(2-hydroxyacetamido)-6-[(1R,2R)-1,2,3-trihydroxypropyl]oxane-2-carboxylic acid (3) (No. 26) (G26)

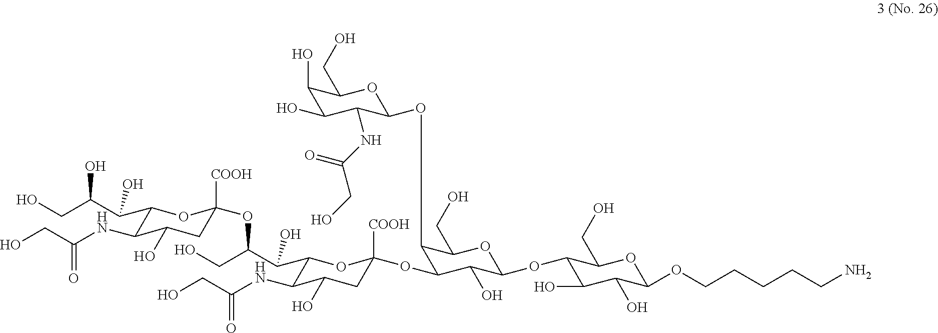 [SMILES: NCCCCCOC1OC(CO)C(OC2OC(CO)C(OC3OC(CO)C(O)C(O)C3NC(=O)CO)C(OC3(C(=O)O)CC(O)C(NC(=O)CO)C([C@H](O)[C@@H](CO)OC4(C(=O)O)CC(O)C(NC(=O)CO)C([C@H](O)[C@H](O)CO)O4)O3)C2O)C(O)C1O]

LiOH (5.0 mmole, 50.0 eq) was added to a stirred solution of 7 (230 mg, 0.1 mmole, 1.00 eq) in 1,4-dioxane (5.00 mL) and H₂O (5.00 mL) at room temperature. After stirring at 80° C. for 36 h, the reaction mixture was evaporated in vacuo. The residue was purified by reverse-phase column chromatography (LiChroprep® RP-18) to give the product residue. NaHCO₃ (2.5 mmole, 25.0 eq) and benzloxyacetyl chloride (2.5 mmole, 25.0 eq) were added to a stirred solution of the above residue in 1,4-dioxane (5.00 mL) and H₂O (5.00 mL) at 0° C. After stirring at the same temperature for 1 h, NaHCO₃ (2.5 mmole, 25.0 eq) and benzloxyacetyl chloride (2.5 mmole, 25.0 eq) were added into the reaction mixture at 0° C. After stirring at the same temperature for 1 h, LiOH (5.0 mmole, 50.0 eq) was added into the reaction mixture. After stirring at the same temperature for 12 h, the reaction mixture was evaporated in vacuo. The residue was purified by reverse-phase column chromatography (LiChroprep® RP-18). To a stirred solution of above residue in 10 mL dry DMF was added NaN₃ (37mg, 0.57 mmol, 5.00 eq) and KI (2 mg, 0.01 mmol, 0.10 eq.) at room temperature. After being stirred at 60° C. overnight, the reaction mixture was evaporated in vacuo. The residue was purified by reverse-phase column chromatography (LiChroprep® RP-18). Pd(OH)₂ (1 mmole) was added to a stirred solution of the above residue in methanol (2.00 mL) and H₂O (2.00 mL). The reaction mixture was hydrogenolyzed for 12 h under H₂ gas atmosphere. The reaction mixture was filtered, and the filtrate was evaporated in vacuo. The residue was purified by reverse-phase column chromatography (LiChroprep® RP-18) to give α(2→8) G_{D2}NGc 3 (53 mg, 0.04 mmol, 42%). ¹H NMR (600 MHz, CDCl₃) δ 4.43 (d, 1H, J=7.9 Hz), 4.39 (d, 1H, J=8.0 Hz), 4.14 (d, 1H, J=7.9 Hz), 4.11 (d, 1H, J=2.9 Hz), 3.98 (d, 1H, J=2.1 Hz), 3.92-3.87 (m, 3H), 3.86-3.78 (m, 6H), 3.75-3.50 (m, 27H), 3.30 (t, 1H, J=9.8 Hz), 3.21 (t, 1H, J=7.5 Hz), 2.90 (t, 1H, J=7.4 Hz), 2.71-2.68 (m, 2H), 2.64-2.61 (m, 1H), 1.72-1.64 (m, 2H), 1.63-1.52 (m, 2H), 1.51-1.47 (m, 2H), 1.39-1.30 (m, 2H); ¹³C NMR (150 MHz, CDCl₃) δ 176.1, 175.7, 175.4, 173.7, 171.0, 102.6, 102.3, 102.0, 100.5, 100.2, 78.5, 78.2, 75.5, 74.8, 74.7, 74.5, 74.3, 74.0, 73.4, 72.8, 72.5, 72.3, 71.8, 70.9, 70.4, 70.0, 69.6, 69.0, 68.5, 68.4, 68.2, 67.9, 67.7, 67.4, 62.5, 61.6, 61.2, 61.0, 60.0, 52.2, 51.4, 40.6, 39.4, 30.9, 28.4, 21.4, 20.0; HRMS (ESI-TOF) Calcd for C₄₇H₈₀N₄O₃₅ [M−H]⁻ 1261.4681, found 1261.4676.

### 5-chloropentyl 4-O-(4-O-(6-O-acetyl-4-O-benzyl-3-O-(2-O-benzoyl-3,4,6-tri-O-benzyl-β-D-galactopyranoside)-2-deoxy-2-(2,2,2-trichloroethyoxy-carbonylamino)-β-D-galactopyranoside)-2-O-benzoyl-6-O-benzyl-3-O-(methyl 7-O-acetyl-5-amino-9-O-benzyl-5-N,4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O-benzyl-β-D-glucopyranoside (6)

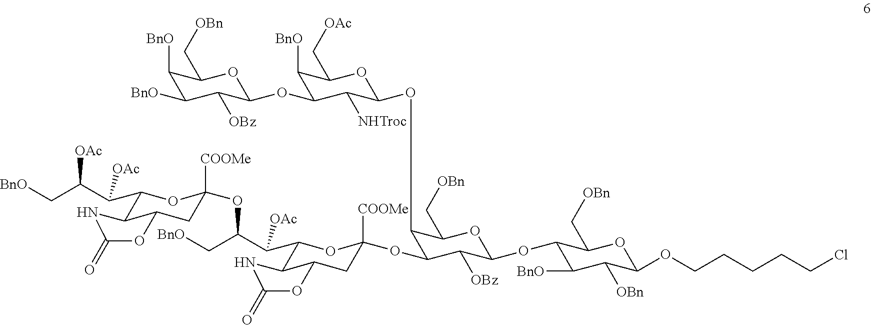

To a stirred solution of 5-chloropentyl 4-O-(4-O-(6-O-acetyl-4-O-benzyl-2-deoxy-2-(2,2,2-trichloroethyoxycarbonylamino)-β-D-galactopyranoside)-2-O-benzoyl-6-O-benzyl-3-O-(methyl 7-O-acetyl-5-amino-9-O-benzyl-5-N4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O-benzyl-β-D-glucopyranoside (7) (340 mg, 0.15 mmol, 1.00 eq.), 4-methylphenyl-2-O-benzoyl-3,4,6-tri-O-benzyl-β-D-galactopyranoside (28) (248 mg, 0.38 mmol, 2.50 eq.) and pulverized activated MS-4 Å in dry CH₂Cl₂ (6 mL) was added N-iodosuccinimide (95 mg, 0.42 mmol, 2.80 eq.) and 0.5 M trifluoromethanesulfonic acid solution in dry Et₂O (90 μL, 0.05 mmol, 0.30 eq.) at −35° C. under argon. After being stirred at the same temperature for 1 h, the reaction mixture was diluted with CH₂Cl₂ and filtered through a pad of celite. The filtrate was poured into a mixture of saturated aq. NaHCO₃ and saturated aq. Na₂S₂O₃. The aqueous layer was extracted with two portions of CH₂Cl₂. The combined extracts were washed with saturated aq. NaHCO₃ and saturated aq. Na₂S₂O₃ and brine, dried over MgSO₄, filtered, and evaporated in vacuo. The residue was chromatographed on silica gel with 50:50 hexane-ethyl acetate to give 5-chloropentyl 4-O-(4-O-(6-O-acetyl-4-O-benzyl-3-O-(2-O-benzoyl-3,4,6-tri-O-benzyl-β-D-galactopyranoside)-2-deoxy-2-(2,2,2-trichloroethyoxy carbonylamino)-β-D-galactopyranoside)-2-O-benzoyl-6-O-benzyl-3-O-(methyl 7-O-acetyl-5-amino-9-O-benzyl-5-N4-O-carbonyl-3,5-dideoxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O-benzyl-β-D-glucopyranoside (6) (386 mg, 0.14 mmol, 93%). ¹H NMR (600 MHz, CDCl₃) δ 8.09 (d, 2H, J=7.3 Hz), 8.04 (d, 2H, J=7.3 Hz), 7.54 (t, 1H, J=7.4 Hz), 7.48 (t, 3H, J=7.4 Hz), 7.41-7.42 (m, 2H), 7.15-7.38 (m, 43H), 7.03-7.09 (m, 4H), 6.90 (d, 2H, J=6.1 Hz), 6.87 (t, 1H, J=7.2 Hz), 5.74 (dd, 1H, J=10.1, 8.0 Hz), 5.67 (br-s, 1H), 5.39 (br-s, 1H), 5.30 (s, 2H), 5.22 (t, 1H, J=9.0 Hz), 5.04-5.12 (m, 4H), 5.00 (d, 1H, J=11.3 Hz), 4.83 (d, 1H, J=11.0 Hz), 4.79 (d, 1H, J=6.6 Hz), 4.75 (d, 1H, J=8.2 Hz), 4.74 (d, 1H, J=10.1 Hz), 4.683 (d, 1H, J=11.3 Hz), 4.679 (d, 1H, J=11.1 Hz), 4.58-4.64 (m, 6H), 4.50 (d, 2H, J=12.4 Hz), 4.46 (s, 2H), 4.44 (d, 1H, J=11.2 Hz), 4.37 (d, 1H, J=12.2 Hz), 4.34 (d, 1H, J=12.1 Hz), 4.31 (d, 1H, J=10.6 Hz), 4.29 (d, 1H, J=12.0 Hz), 4.221 (d, 1H, J=8.1 Hz), 4.217 (d, 1H, J=9.6 Hz), 4.18 (d, 1H, J=10.6 Hz), 4.00-4.10 (m, 6H), 3.73 (s, 3H), 3.69 (s, 3H), 3.67-3.84 (m, 8H), 3.44 (t, 2H, J=6.6 Hz), 3.41-3.61 (m, 14H), 3.20-3.31 (m, 5H), 2.98 (t, 1H, J=10.1 Hz), 2.92 (dd, 1H, J=11.9, 3.0 Hz), 2.88 (dd, 1H, J=12.0, 3.3 Hz), 2.48 (t, 1H, J=10.3 Hz), 2.17 (s, 3H), 2.16 (t, 1H, J=11.6 Hz), 1.99 (t, 1H, J=12.8 Hz), 1.90 (s, 3H), 1.80 (s, 3H), 1.74 (s, 3H), 1.71-1.76 (m, 2H), 1.56-1.64 (m, 2H), 1.43-1.53 (m, 3H); ¹³C NMR (150 MHz, CDCl₃) δ 171.3, 170.5, 170.3, 170.2, 168.2, 168.0, 165.4, 164.7, 159.3, 159.0, 153.8, 138.8, 138.74, 138.73, 738.369, 138.61, 138.5, 138.0, 137.7, 137.3, 136.6, 133.4, 133.1, 130.1, 130.0, 129.4, 129.0, 128.71, 128.68, 128.59, 128.55, 128.5, 128.4, 128.34, 128.31, 128.29, 128.26, 128.2, 128.1, 128.0, 127.9, 127.8, 127.74, 128.69, 127.64, 127.60, 127.53, 127.48, 127.4, 103.5, 101.9, 100.7, 100.5, 98.6, 98.2, 96.7, 82.9, 82.1, 79.8, 76.7, 76.6, 76.3, 76.2, 75.5, 75.4, 75.3, 75.0, 74.9, 74.8, 74.6, 74.29, 74.26, 74.1, 73.9, 73.8, 73.73, 73.69, 73.6, 73.2, 73.1, 72.1, 72.0, 71.7, 71.4, 71.1, 69.6, 68.8, 68.71, 68.65, 68.2, 67.7, 67.6, 63.0, 58.9, 57.8, 55.4, 53.4, 53.2, 45.0, 37.4, 37.0, 32.5, 29.1, 23.7, 21.6, 20.8, 20.5; HRMS (ESI-TOF) Calcd for C₁₄₆H₁₅₇N₃O₄₄Cl₄Na₂ [M+2Na]²⁺ 1421.9423, found 1421.9431.

### 5-aminopentyl 4-O-(4-O-(2-acetoamino-3-O-(β-D-galactopyranoside)-2-deoxy-β-D-galactopyranoside)-3-O-(5-acetoamino-3,5-dideoxy-8-O-(5-acetoamino-3,5-dideoxy-D-glycero-α-D-galacto-2-nonulopyranosylonate)-D-glycero-α-D-galacto-2-nonulopyranoylonate)-β-D-galactopyranoside)-β-D-glucopyranoside (5) (No. 20) (G20)

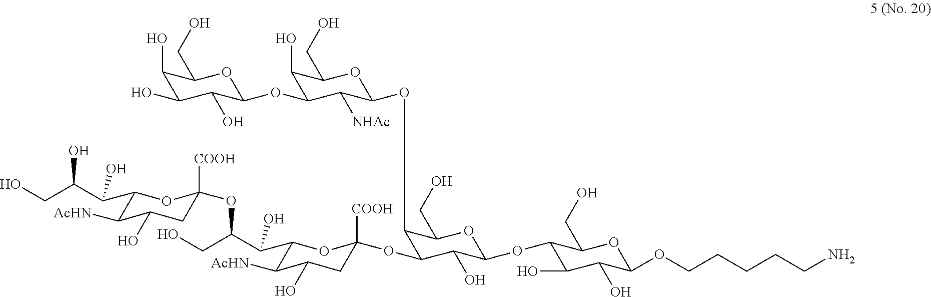 [SMILES: CC(=O)NC1C(OC2C(CO)OC(OC3C(CO)OC(OCCCCCN)C(O)C3O)C(O)C2OC2(C(=O)O)CC(O)C(NC(C)=O)C([C@H](O)[C@@H](CO)OC3(C(=O)O)CC(O)C(NC(C)=O)C([C@H](O)[C@H](O)CO)O3)O2)OC(CO)C(O)C1OC1OC(CO)C(O)C(O)C1O]

To a stirred solution of 5-chloropentyl 4-O-(4-O-(6-O-acetyl-4-O-benzyl-3-O-(2-O-benzoyl-3,4,6-tri-O-benzyl-β-D-galactopyranoside)-2-deoxy-2-(2,2,2-trichloroethyoxycarbonylamino)-β-D-galactopyranoside)-2-O-benzoyl-6-O-benzyl-3-O-(methyl 7-O-acetyl-5-amino-9-O-benzyl-5-N4-O-carbonyl-3,5-dide-oxy-8-O-(methyl 5-amino-9-O-benzyl-5-N4-O-carbonyl-7,8-O-diacetyl-3,5-dideoxy-D-glycero-α-D-galacto-2-nonulopyranosylonate)-D-glycero-α-D-galacto-2-nonulopyranoylonate)-β-D-galactopyranoside)-2,3,6-tri-O-benzyl-β-D-glucopyranoside (6) (156 mg, 0.056 mmol, 1.00 eq.) in 12 mL THF was added 6 mL 1M aq. NaOH at room temperature. After being stirred at 80° C. overnight, the reaction mixture was evaporated in vacuo. The residue was purified by reverse-phase column chromatography (LiChroprep® RP-18). To a stirred solution of above residue in a mixture of 6.0 mL 1,4-dioxane and 6.0 mL H₂O was added NaHCO₃ (400 mg) and acetic anhydride (200 μL) at room temperature. After being stirred at the same temperature for 1 h, the reaction mixture was added NaHCO₃ (400 mg) and acetic anhydride (200 μL). After being stirred at the same temperature for another 1 h, the reaction mixture was added LiOH (400 mg) at room temperature. After being stirred at the same temperature for 12 h, the reaction mixture was evaporated in vacuo. The residue was purified by reverse-phase column chromatography (LiChroprep® RP-18). To a stirred solution of above residue in 8 mL dry DMF was added NaN₃ (18 mg, 0.277 mmol, 5.00 eq) and KI (1 mg, 0.006 mmol, 0.10 eq.) at room temperature. After being stirred at 60° C. overnight, the reaction mixture was evaporated in vacuo. The residue was purified by reverse-phase column chromatography (LiChroprep® RP-18). To a stirred solution of above residue in a mixture of 8.0 mL methanol and 8.0 mL H₂O was added cat. AcOH and Pd(OH)₂ (400 mg) at room temperature. After being stirred at room temperature under H₂ gas atmosphere overnight, the reaction mixture was filtered through a pad of celite and the filtrate was evaporated in vaccuo. The residue was purified by reverse-phase column chromatography (LiChroprep® RP-18) to give 5-aminopentyl 4-O-(4-O-(2-acetoamino-3-O-(β-D-galactopyra-noside)-2-deoxy-β-D-galactopyranoside)-3-O-(5-acetoamino-3,5-dideoxy-8-O-(5-acetoamino-3,5-dideoxy-D-glycero-α-D-galacto-2-nonulopyranosylonate)-D-glycero-α-D-galacto-2-nonulopyranoylonate)-β-D-galactopyranoside)-β-D-glucopyranoside (5) (37 mg, 0.027 mmol, 48%). ¹H NMR (600 MHz, CDCl₃) δ 4.74 (d, 1H, J=8.4 Hz), 4.52 (d, 1H, J=10.1 Hz), 4.50 (d, 1H, J=10.3 Hz), 4.48 (d, 1H, J=8.0 Hz), 4.15-4.19 (m, 3H), 4.09-4.11 (m, 1H), 4.05 (d, 1H, J=2.3 Hz), 3.98-4.02 (m, 2H), 3.88-3.95 (m, 5H), 3.57-3.86 (m, 28H), 3.52 (dd, 1H, J=9.9, 7.9 Hz), 3.39 (dd, 1H, J=9.8, 8.0 Hz), 3.29 (t, 1H, J=8.6 Hz), 3.00 (t, 2H, J=7.6 Hz), 2.76 (dd, 1H, J=12.3, 4.6 Hz), 2.68 (dd, 1H, J=12.2, 4.2 Hz), 2.07 (s, 3H), 2.03 (s, 3H), 2.02 (s, 3H), 1.78 (t, 1H, J=12.2 Hz), 1.73 (t, 1H, J=12.2 Hz), 1.63-1.71 (m, 4H), 1.43-1.48 (m, 2H); ¹³C NMR (150 MHz, CDCl₃) δ 174.93, 174.90, 174.8, 173.4, 173.3, 104.6, 102.7, 102.4, 102.0, 100.53, 100.45, 79.8, 78.3, 78.2, 75.8, 74.9, 74.8, 74.5, 74.31, 74.29, 74.1, 73.7, 72.7, 72.6, 72.4, 71.7, 70.6, 70.0, 69.6, 69.2, 68.54, 68.46, 68.08, 68.06, 67.8, 62.5, 61.4, 60.9, 60.8, 60.6, 59.9, 52.3, 51.7, 51.3, 40.4, 39.3, 39.1, 28.1, 26.4, 22.5, 22.3, 22.01, 21.98; HRMS (ESI-TOF) Calcd for C₅₃H₈₉N₄O₃₇ [M−H]6⁻ 1373.5206, found 1373.5201.

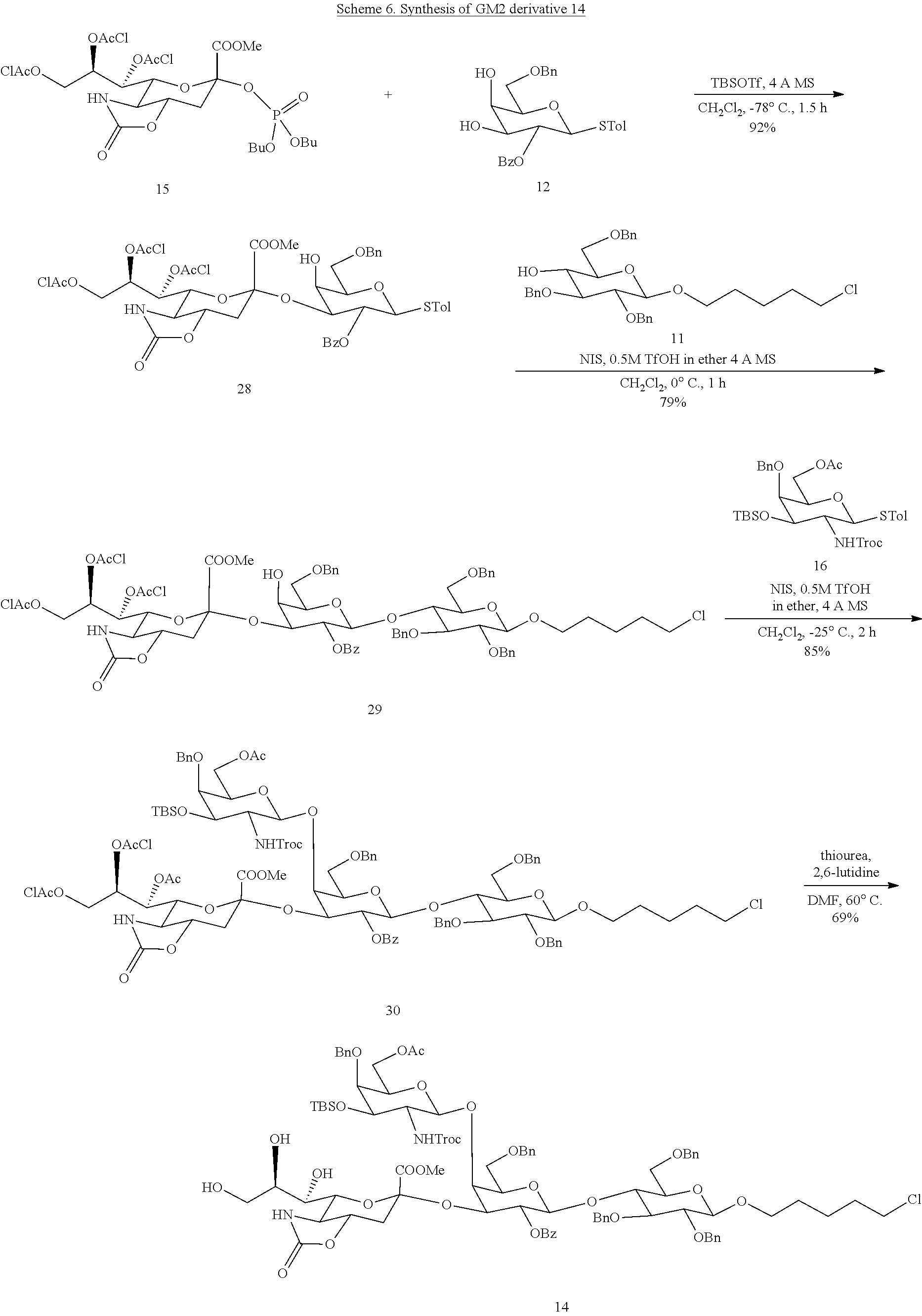

### Methyl 4-((1S,2R)-1,2,3-tris (2-chloroacetoxy)propyl)-6-(3-(benzoyloxy)-6-(benzyloxymethyl)-5-hydroxy-2-(p-tolylthio)-tetrahydro-2H-pyran-4-yloxy)-2-oxo-hexahydro-2H-pyrano[3,4-d]oxazole-6-carboxylate (28)

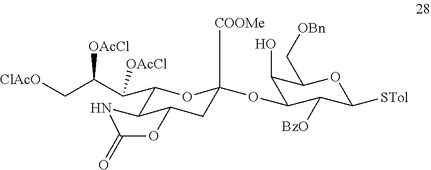

To a stirred solution of methyl 4-((1S,2R)-1,2,3-tris (2-chloroacetoxy) propyl)-6-(dibutoxyphosphoryl-oxy)-2-oxo-hexahydro-2H-pyrano[3,4-d]oxazole-6-car-boxylate (15) (2.65 g, 3.64 mmol, 1.30 eq.), 4-methylphenyl 2-O-benzoyl-6-O-benzyl-1-thio-β-D-galactopyranoside (12) (1.34 g, 2.80 mmol, 1.00 eq.) and pulverized activated MS-4 Å in dry CH₂Cl₂ (50 mL) was added TBSOTf (0.960 mL, 4.19 mmol, 1.50 eq.) at −78° C. under argon. After being stirred at the same temperature for 1.5 h, the reaction mixture was neutralized with triethylamine and filtered through a pad of celite. The filtrate mixture was poured into a mixture of saturated aq. NaHCO₃ and saturated aq. Na₂S₂O₃. The aqueous layer was extracted with two portions of CH₂Cl₂. The combined extracts were washed with brine, dried over MgSO₄, filtered, and evaporated in vacuo. The residue was chromatographed on silica gel with 60:40 hexane-ethyl acetate to give 28 (2.57 g, 2.57 mmol, 92%, α only). The α/β ratio was determined by ¹H NMR analysis. ¹H NMR (600 MHz, CDCl₃) δ 8.11 (d, 2H, J=7.2 Hz), 7.63 (t, 1H, J=7.7 Hz), 7.51 (t, 2H, J=7.5 Hz), 7.25-7.32 (m, 7H), 7.02 (d, 2H, J=7.8 Hz), 5.67 (dd, 1H, J=10.3, 1.5 Hz), 5.27 (t, 1H, J=9.7 Hz), 5.15 (br-s, 1H), 5.05 (d, 1H, J=9.6 Hz), 4.86 (d, 1H, J=10.2 Hz), 4.56 (s, 2H), 4.41 (d, 1H, J=1.4 Hz), 4.39 (d, 1H, J=1.8 Hz), 4.28-4.32 (m, 1H), 4.14 (d, 1H, J=1.8 Hz), 4.11-4.13 (m, 1H), 4.04 (d, 1H, J=2.0 Hz), 4.02 (s, 2H), 3.78-3.82 (m, 4H), 3.66-3.76 (m, 1H), 3.68 (s, 3H), 3.28 (s, 2H), 2.84-2.90 (m, 2H), 2.59 (br-s, 1H), 2.28 (s, 3H), 1.99 (t, 1H, J=12.6 Hz); ¹³C NMR (150 MHz, CDCl₃) δ 168.8, 168.2, 167.3, 167.0, 158.9, 138.5, 138.2, 133.5, 130.5, 130.3, 129.8, 128.8, 128.6, 128.5, 128.0, 127.8, 98.8, 86.6, 76.7, 76.5, 75.9, 73.8, 73.5, 70.0, 69.3, 69.2, 67.8, 67.6, 63.3, 57.3, 53.7, 41.5, 40.6, 39.7, 37.0, 21.4; HRMS (ESI-TOF) Calcd for C₄₄H₄₆Cl₃NO₁₇SNa [M+Na]⁺ 1020.1450, found 1020.1442.

### Methyl 4-((1S,2R)-1,2,3-tris(2-chloroacetoxy)propyl)-6-(2-(4,5-bis(benzyl-oxy)-2-(benzyloxymethyl)-6-(5-chloropentyloxy)-tetrahydro-2H-pyran-3-yloxy)-3-(benzoyloxy)-6-(benzyloxymethyl)-5-hydroxy-tetrahydro-2H-pyran-4-yloxy)-2-oxo-hexahydro-2H-pyrano[3,4-d]oxazole-6-carboxylate (29)

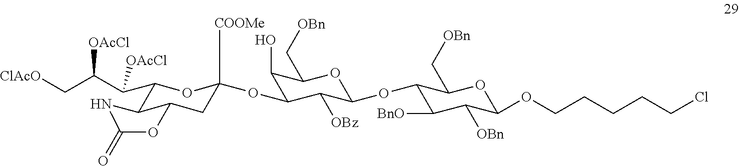

To a stirred solution of methyl 4-((1S,2R)-1,2,3-tris (2-chloroacetoxy) propyl)-6-(3-(benzoyloxy)-6-(benzyloxymethyl)-5-hydroxy-2-(p-tolylthio)-tetrahydro-2H-pyran-4-yloxy)-2-oxo-hexahydro-2H-pyrano[3,4-d]oxazole-6-carboxylate (28) (1.54 g, 1.31 mmol, 1.50 eq.), 5-chloropentyl-2,3,6-tri-O-benzyl-β-D-glucopyranoside (11) (1.37 g, 2.47 mmol, 2.00 eq.) and pulverized activated MS-4 Å in dry CH₂Cl₂ (30 mL) was added N-iodosuccinimide (0.59 g, 2.62 mmol, 2.00 eq.) and 0.5 M trifluoromethanesulfonic acid solution in dry Et₂O (0.79 mL, 0.39 mmol, 0.30 eq.) at 0° C. under argon. After being stirred at the same temperature for 1 h, the reaction mixture was diluted with CH₂Cl₂ and filtered through a pad of celite. The filtrate was poured into a mixture of saturated aq. NaHCO₃ and saturated aq. Na₂S₂O₃. The aqueous layer was extracted with two portions of CH₂Cl₂. The combined extracts were washed with saturated aq. NaHCO₃ and saturated aq. Na₂S₂O₃ and brine, dried over MgSO₄, filtered, and evaporated in vacuo. The residue was chromatographed on silica gel with 50:50 hexane-ethyl acetate to give 29 (1.48 g, 1.31 mmol, 79%). ¹H NMR (600 MHz, CDCl₃) δ 8.08 (d, 2H, J=7.2 Hz), 7.61 (t, 1H, J=7.5 Hz), 7.43 (t, 2H, J=7.8 Hz), 7.35 (d, 2H, J=7.2 Hz), 7.20-7.29 (m, 18H), 5.64 (dd, 1H, J=10.1, 1.4 Hz), 5.32 (t, 1H, J=9.5 Hz), 5.15 (s, 1H), 5.06 (d, 1H, J=2.0 Hz), 4.97 (d, 1H, J=7.9 Hz), 4.93 (d, 1H, J=10.9 Hz), 4.83 (d, 1H, J=11.4 Hz), 4.66 (d, 1H, J=10.8 Hz), 4.14-4.39 (m, 2H), 4.33 (d, 2H, J=11.4 Hz), 4.24-4.29 (m, 3H), 4.13 (d, 2H, J=15.6 Hz), 4.05-4.14 (m, 3H), 3.99 (s, 2H), 3.81-3.88 (m, 2H), 3.75 (t, 1H, J=8.3 Hz), 3.65 (s, 3H), 3.62-3.66 (m, 2H), 3.55-3.58 (m, 3H), 3.442 (s, 2H), 3.44 (d, 2H, J=11.4 Hz), 3.28-3.41 (m, 4H), 2.87 (t, 1H, J=10.5 Hz), 2.80 (dd, 1H, J=11.3, 2.1 Hz), 2.54 (br-s, 1H), 2.03 (t, 1H, J=12.5 Hz), 1.71-1.76 (m, 2H), 1.57-1.64 (m, 2H), 1.41-1.49 (m, 2H); ¹³C NMR (150 MHz, CDCl₃) δ 168.5, 168.1, 167.3, 166.9, 164.9, 158.9, 139.3, 138.7, 138.6, 138.3, 133.6, 130.2, 130.1, 128.9, 128.51, 128.47, 128.46, 128.4, 128.1, 127.9, 127.8, 127.70, 127.65, 127.6, 127.4, 103.6, 100.7, 99.2, 83.1, 82.2, 76.9, 76.6, 75.4, 75.3, 74.9, 74.6, 73.51, 73.47, 73.3, 72.3, 72.0, 70.1, 69.8, 69.1, 68.1, 67.7, 67.0, 63.3, 57.3, 53.7, 45.1, 41.3, 40.5, 39.7, 36.8, 36.5, 32.5, 29.2, 24.9, 23.7; HRMS (ESI-TOF) Calcd for C₆₉H₇₇Cl₄NO₂₃Na [M+Na]⁺ 1450.3538, found 1450.3535.

### Methyl 6-(2-(4,5-bis (benzyloxy)-2-(benzyloxymethyl)-6-(5-chloropentyl-oxy)-tetrahydro-2H-pyran-3-yloxy)-5-(6-(acetoxymethyl)-5-(benzyloxy)-4-(tert-butyldimethylsilyloxy)-3-((2,2,2-trichloroethoxy) carbonyl)-tetrahydro-2H-pyran-2-yloxy)-3-(benzoyloxy)-6-(benzyloxymethyl)-tetrahydro-2H-pyran-4-yloxy)-2-oxo-4-((1R,2R)-1,2,3-trihydroxypropyl)-hexahydro-2H-pyrano[3,4-d]oxazole-6-carboxylate (14)

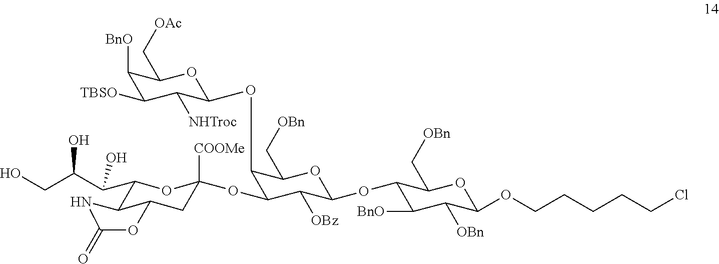

To a stirred solution of methyl 4-((1S,2R)-1,2,3-tris (2-chloroacetoxy) propyl)-6-(2-(4,5-bis (benzyloxy)-2-(benzyloxymethyl)-6-(5-chloropentyloxy)-tetrahydro-2H-pyran-3-yloxy)-3-(benzoyloxy)-6-(benzyloxymethyl)-5-hydroxy-tetrahydro-2H-pyran-4-yloxy)-2-oxo-hexahydro-2H-pyrano[3,4-d]oxazole-6-carboxylate (29) (1.78 g, 1.24 mmol, 1.00 eq.), 4-methylphenyl 6-O-acetyl-4-O-benzyl-3-O-tert-butyldimethylsilyl-2-deoxy-1-thio-2-(2,2,2-trichloroethyoxycarbamoyl)-β-D-galactopyranoside (16) (1.32 g, 1.87 mmol, 1.50 eq.) and pulverized activated MS-4 Å in dry CH₂Cl₂ (30 mL) was added N-iodosuccinimide (0.56 g, 2.49 mmol, 2.00 eq.) and 0.5 M trifluoromethanesulfonic acid solution in dry Et₂O (0.75 mL, 0.37 mmol, 0.30 eq.) at −25° C. under argon. After being stirred at the same temperature for 2 h, the reaction mixture was diluted with CH₂Cl₂ and filtered through a pad of celite. The filtrate was poured into a mixture of saturated aq. NaHCO₃ and saturated aq. Na₂S₂O₃. The aqueous layer was extracted with two portions of CH₂Cl₂. The combined extracts were washed with saturated aq. NaHCO₃ and saturated aq. Na₂S₂O₃ and brine, dried over MgSO₄, filtered, and evaporated in vacuo. The residue was chromatographed on silica gel with 55:45 hexane-ethyl acetate to give product (2.13 g, 1.06 mmol, 85%). To a stirred solution of product (0.34 g, 0.17 mmol, 1.00 eq.) in DMF (7 mL) was added thiourea (80.0 mg, 1.01 mmol, 6.00 eq.) and 2,6-lutidine (60 μL, 0.51 mmol, 3.00 eq.) at room temperature. After being stirred at 60° C. for 5 h, the reaction mixture was poured into ice-cooled 1 M HCl. The aqueous layer was extracted with two portions of ethyl acetate. The combined extracts were washed with saturated aq. NaHCO₃ and saturated aq. Na₂S₂O₃ and brine, dried over MgSO₄, filtered, and evaporated in vacuo. The residue was chromatographed on silica gel with 55:45 hexane-ethyl acetate to give 14 (0.21 g, 0.12 mmol, 69%). ¹H NMR (600 MHz, CDCl₃) δ 7.85 (d, 2H, J=7.6 Hz), 7.51 (t, 1H, J=7.4 Hz), 744-7.17 (m, 25H), 7.14 (d, 2H, J=7.6 Hz), 6.71 (t, 1H, J=7.4 Hz), 6.15 (br-s, 2H), 5.22 (t, 1H, J=7.9 Hz), 5.12 (d, 1H, J=11.0 Hz), 4.91 (m, 2H), 4.85-4.72 (m, 6H), 4.64 (d, 1H, J=12.0Hz), 4.57 (d, 1H, J=11.3 Hz), 4.52 (d, 1H, J=12.6 Hz), 4.27 (d, 1H, J=12.3 Hz), 4.24 (t, 1H, J=4.5 Hz), 4.23 (d, 1H, J=12.3 Hz), 4.04-4.02 (m, 3H), 3.84 (t, 1H, J-9.8 Hz), 3.82 (s, 3H), 3.80-3.70 (m, 5H), 3.67-3.65 (m, 2H), 3.60-3.57 (m, 5H), 3.51 (t, 1H, J=9.0 Hz), 3.49-3.47 (m, 2H), 3.43 (t, 2H, J=6.7 Hz), 3.42-3.38 (m, 3H), 3.24 (t, 2H, J=8.9 Hz), 3.11 (d, 1H, J=9.5 Hz), 2.44 (d, 1H, J=9.1 Hz), 2.33 (t, 1H, J=12.4 Hz), 1.97 (s, 3H), 1.73-1.9 (m, 2H), 1.59-1.63 (m, 2H), 1.49-1.42 (m, 2H), 0.90 (s, 9H), 0.17 (s, 3H), 0.14 (s, 3H); ¹³C NMR (150 MHz, CDCl₃) 8171.3, 169.7, 164.1, 160.1, 154.1, 138.8, 138.6, 138.5, 138.4, 138.3, 133.5, 129.6, 129.2, 129.0, 128.7, 128.5, 128.4, 128.3, 128.2, 128.1, 128.0, 128.8, 127.7, 127.5, 127.4, 127.3, 103.5, 102.6, 101.3, 100.1, 96.0, 82.3, 81.4, 76.4, 76.0, 75.7, 75.0, 74.9, 74.8, 74.4, 73.5, 73.4, 71.3, 71.2, 71.0, 69.5, 69.0, 67.8, 63.6, 62.7, 57.3, 54.9, 54.4, 44.9, 32.3, 31.9, 30.0, 29.7, 29.6, 29.4, 28.9, 27.0, 10 25.8, 23.3, 22.7, 20.9, 18.0, 14.1, −4.1, −5.0; HRMS (ESI-TOF) Calcd for C₈₇H₁₀₈Cl₄N₂O₂₇SiNa [M+Na]⁺ 1803.5561, found 1803.5553.

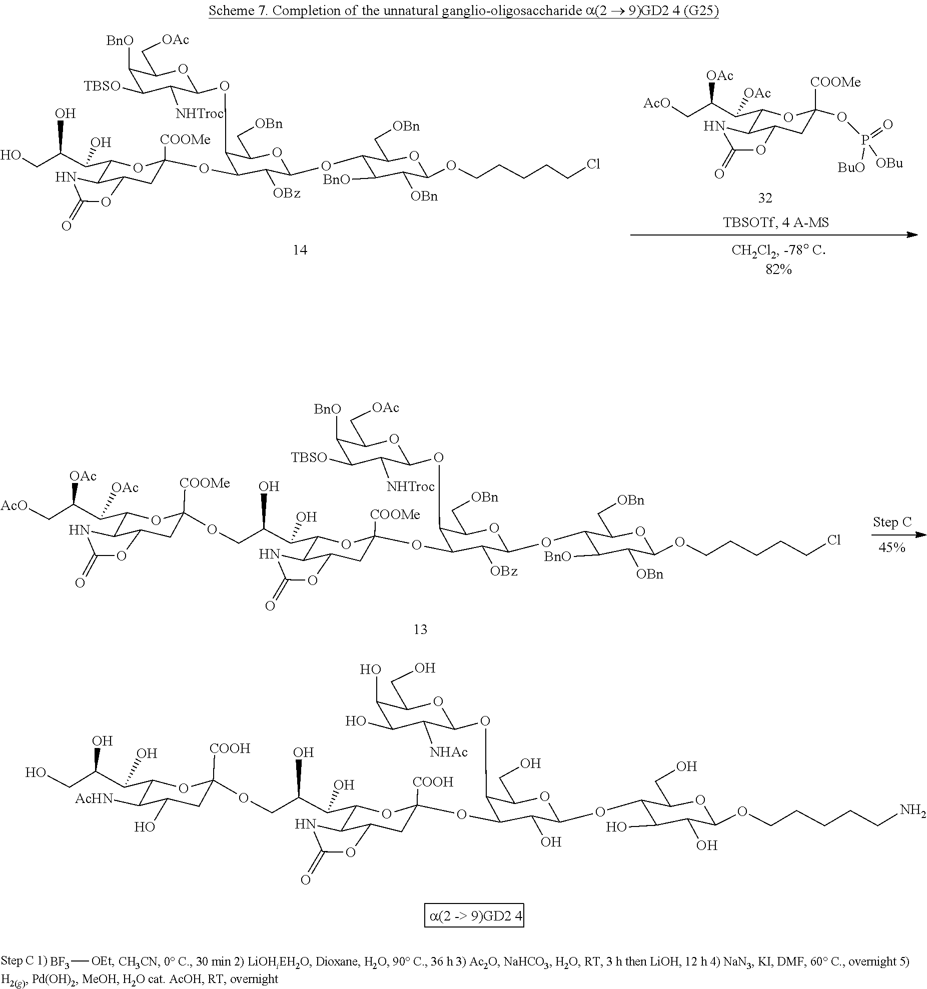

### (1S,2R)-1-((3aR,4R,6R,7aS)-6-((2R,3R)-3-((3aR,4R,6S,7aS)-6-(((2R,3S,4S,5R,6S)-3-(((2S,3R,4R,5S,6R)-6-(acetoxymethyl)-5-(benzyloxy)-4-((tert-butyldimethylsilyl) oxy)-3-(((2,2,2-trichloroethoxy)carbonyl)amino)tetrahydro-2H-pyran-2-yl)oxy)-5-(benzoyloxy)-2-((benzyloxy)methyl)-6-(((2R,3R,5R,6R)-4,5-bis (benzyloxy)-2-((benzyloxy) methyl)-6-((5-chloropentyl)oxy)tetrahydro-2H-pyran-3-yl)oxy)tetrahydro-2H-pyran-4-yl) oxy)-6-(methoxycarbonyl)-2-oxohexahydro-2H-pyrano[3,4-d]oxazol-4-yl)-2,3-dihydroxypropoxy)-6-(methoxycarbonyl)-2-oxohexahydro-2H-pyrano[3,4-d]oxazol-4-yl) propane-1,2,3-triyltriacetate (13)

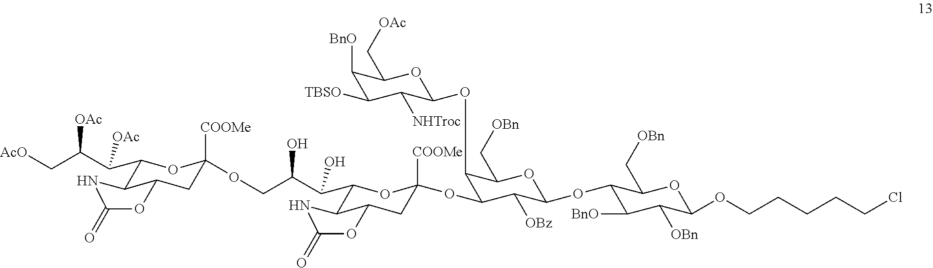

To a stirred solution of methyl 6-(2-(4,5-bis(benzyloxy)-2-(benzyl oxymethy 1)-6-(5-chloropentyloxy)-tetrahydro-2H-pyran-3-yloxy)-5-(6-(acetoxymethyl)-5-(benzyloxy)-4-(tert-butyldimethylsilyloxy)-3-((2,2,2-trichloroethoxy)carbonyl)-tetrahydro-2H-pyran-2-yloxy)-3-(benzoyloxy)-6-(benzyloxymethyl)-tetrahydro-2H-pyran-4-yloxy)-2-oxo-4-((1R,2R)-1,2,3-trihydroxypropyl)-hexahydro-2H-pyrano[3,4-d]oxazole-6-carboxylate (14) (240 mg, 0.13 mmol, 1.00 eq.), methyl 6-(dibutoxyphosphoryloxy)-2-oxo-4-((1S,2R)-1,2,3-triacetoxypropyl)-hexahy-dro-2H-pyrano[3,4-d]oxazole-6-carboxylate (32) (130 mg, 0.20 mmol, 1.50 eq.) and pulverized activated MS-4 Å in dry CH₂Cl₂ (10 mL) was added TBSOTf (47 μL, 0.20 mmol, 1.50 eq.) at −78° C. under argon. After being stirred at the same temperature for 1.5 h, the reaction mixture was neutralized with saturated aq. NaHCO₃ and filtered through a pad of celite. The filtrate mixture was poured into a mixture of saturated aq. NaHCO₃. The aqueous layer was extracted with two portions of CH₂Cl₂. The combined extracts were washed with brine, dried over MgSO₄, filtered, and evaporated in vacuo. The residue was chromatographed on silica gel with 40:60 hexane-ethyl acetate to give 13 (240 mg, 0.11 mmol, 82%, α/β=>95/5). The α/β ratio was determined by ¹H NMR analysis. ¹H NMR (600 MHz, CDCl₃) δ 7.74 (d, 2H, J=7.8 Hz), 7.58 (t, 1H, J=7.4 Hz), 7.47-7.13 (m, 27H), 6.66 (t, 1H, J=7.3 Hz), 6.06 (br-s, 1H), 5.47 (t, 1H, J=8.7 Hz), 5.30 (dt, 1H, J=9.9, 2.2 Hz), 5.21 (t, 1H, J=8.7 Hz), 5.12 (d, 1H, J=11.0 Hz), 5.01 (d, 1H, J=10.3 Hz), 4.94-4.92 (m, 2H), 4.85 (d, 1H, J=11.8 Hz), 4.80 (br-s, 2H), 4.73 (t, 2H, J=10.7 Hz), 4.65 (d, 1H, J=8.18 Hz), 4.58 (d, 1H, J=9.0 Hz), 4.54 (d, 1H, J=11.0 Hz), 4.49-4.46 (m, 2H), 4.29 (d, 1H, J=10.5 Hz), 4.225 (dd, 1H, J=9.9, 1.5 Hz), 4.21-4.16 (m, 5H), 3.97 (d, 1H, J=3.0 Hz), 3.94-3.86 (m, 5H), 3.85 (s, 3H), 3.82 (s, 3H), 3.81-3.77 (m, 3H), 3.71-3.69 (m, 4H), 3.66 (d, 1H, J=1.9 Hz), 3.61-3.59 (m, 2H), 3.52-3.48 (m, 3H), 3.42 (t, 2H, J=6.8 Hz), 3.38-3.31 (m, 3H), 3.20 (t, 1H, J=9.0 Hz), 3.06 (d, 1H, J=9.7 Hz), 3.02 (t, 1H, J=10.2 Hz), 2.91 (dd, 1H, J=12.2, 3.4 Hz), 2.46 (dd, 1H, J=11.7, 3.0 Hz), 2.22 (s, 3H), 2.19 (s, 3H), 1.97 (s, 3H), 1.92 (s, 3H), 1.74-1.69 (m, 2H), 1.59-1.55 (m, 2H), 1.49-1.42 (m, 2H), 0.89 (s, 9H), 0.17 (s, 3H), 0.12 (s, 3H); ¹³C NMR (150 MHz, CDCl₃) δ 172.3, 171.8, 171.7, 170.6, 167.9, 167.6, 164.3, 160.3, 154.2, 139.1, 138.9, 138.8, 138.7, 137.5, 136.3, 132.6, 130.2, 129.3, 128.9, 128.7, 128.6, 128.5, 128.4, 128.2, 128.1, 128.0, 127.9, 127.8, 127.7, 127.6, 127.5, 102.5, 101.6, 100.7, 100.1, 96.3, 83.6, 81.0, 78.6, 76.5, 76.2, 76.1, 75.2, 75.1, 75.0, 74.9, 74.5, 74.2, 74.1, 73.6, 73.5, 73.4, 73.3, 71.9, 71.2, 70.9, 69.8, 69.1, 68.5, 68.1, 67.2, 64.4, 59.2, 57.1, 55.9, 53.2, 53.0, 44.1, 38.5, 35.6, 34.5, 29.8, 26.6, 23.7, 21.7, 20.9, 20.5, 20.2, 19.2, −3.8, −5.1; HRMS (ESI-TOF) Calcd for C₁₀₄H₁₂₉Cl₄N₃O₃₈SiNa [M+Na]⁺ 2218.6675, found 2218.6675.

### (2S,4S,5R,6R)-5-acetamido-6-((1R,2R)-3-(((2R,4S,5R,6R)-5-acetamido-2-carboxy-4-hydroxy-6-((1R,2R)-1,2,3-trihydroxypropyl)tetrahydro-2H-pyran-2-yl) oxy)-1,2-dihydroxypropyl)-2-(((2R,3S,4R,5R,6S)-3-(((2S,3R,4R,5R,6R)-3-acetamido-4,5-dihydroxy-6-(hydroxymethyl)tetrahydro-2H-pyran-2-yl) oxy)-6-(((2R,3S,5R,6R)-6-((5-aminopentyl)oxy)-4,5-dihydroxy-2-(hydroxymethyl)tetrahydro-2H-pyran-3-yl)oxy)-5-hydroxy-2-(hydroxymethyl)tetrahydro-2H-pyran-4-yl) oxy)-4-hydroxytetrahydro-2H-pyran-2-carboxylic acid (4) (No. 25) (G25)

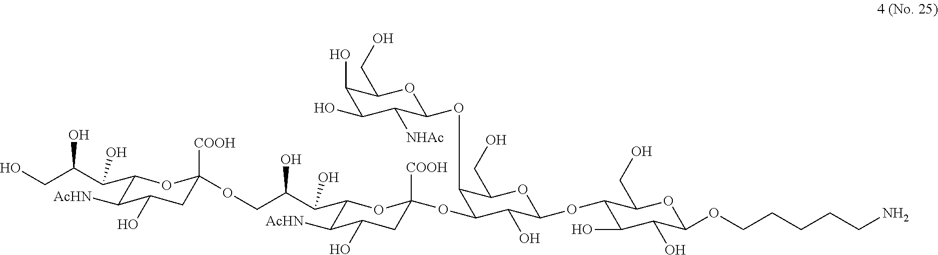 [SMILES: CC(=O)NC1C(OC2C(CO)OC(OC3C(CO)OC(OCCCCCN)C(O)C3O)C(O)C2OC2(C(=O)O)CC(O)C(NC(C)=O)C([C@H](O)[C@H](O)COC3(C(=O)O)CC(O)C(NC(C)=O)C([C@H](O)[C@H](O)CO)O3)O2)OC(CO)C(O)C1O]

To a stirred solution of (1S,2R)-1-((3aR,4R,6R,7aS)-6-((2R,3R)-3-((3aR,4R,6S,7aS)-6-(((2R,3S,4S,5R,6S)-3-(((2S,3R,4R,5S,6R)-6-(acetoxymethyl)-5-(benzyloxy)-4-((tert-butyldimethylsilyl)oxy)-3-(((2,2,2-trichloroethoxy)carbonyl)amino)tetrahydro-2H-pyran-2-yl)oxy)-5-(benzoyloxy)-2-((benzyloxy)methyl)-6-(((2R,3R,5R,6R)-4,5-bis(benzyloxy)-2-((benzyloxy)methyl)-6-((5-chloropentyl)oxy)tetrahydro-2H-pyran-3-yl)oxy)tetrahydro-2H-pyran-4-yl)oxy)-6-(methoxycarbonyl)-2-oxohexahydro-2H-pyrano[3,4-d]oxazol-4-yl)-2,3-dihydroxypropoxy)-6-(methoxycarbonyl)-2-oxohexahydro-2H-pyrano[3,4-d]oxazol-4-yl)propane-1,2,3-triyl triacetate (13) (210 mg, 0.10 mmol, 1.00 eq.) in dry acetonitrile (10 mL) was added 48% BF₃·OEt₂ (75 μL, 0.57 mmol, 6.00 eq.) at 0° C. under argon. After being stirred at the same temperature for 30 min, the reaction mixture was poured into saturated aq. NaHCO₃. The aqueous phase was washed with two portions of ethyl acetate. The combined extracts were washed with brine, dried over MgSO₄, filtered, and evaporated in vacuo. LiOH (5.0 mmole, 50.0 eq) was added to a stirred solution of the residue (170 mg, 0.1 mmole, 1.00 eq) in 1,4-dioxane (5.00 mL) and H₂O (5.00 mL) at room temperature. After stirring at 80° C. for 36 h, the reaction mixture was evaporated in vacuo. The residue was purified by reverse-phase column chromatography (LiChroprep® RP-18) to give the product residue. NaHCO₃ (5.0 mmole, 50.0 eq) and acetic anhydride (5.0 mmole, 50.0 eq) were added to a stirred solution of the above residue in H₂O (3.00 mL) at room temperature. After stirring at the same temperature for 1 h, NaHCO₃ (5.0 mmole, 50.0 eq) and acetic anhydride (5.0 mmole, 50.0 eq) were added into the reaction mixture at room temperature. After stirring at the same temperature for 1 h, LiOH (5.0 mmole, 50.0 eq) was added into the reaction mixture. After stirring at the same temperature for 12 h, the reaction mixture was evaporated in vacuo. The residue was purified by reverse-phase column chromatography (LiChroprep® RP-18). To a stirred solution of above residue in 10 mL dry DMF was added NaN₃ (37 mg, 0.57 mmol, 5.00 eq) and KI (2 mg, 0.01 mmol, 0.10 eq.) at room temperature. After being stirred at 60° C. overnight, the reaction mixture was evaporated in vacuo. The residue was purified by reverse-phase column chromatography (LiChroprep® RP-18). Pd(OH)₂ (1 mmole) was added to a stirred solution of the above residue in methanol (2.00 mL) and H₂O (2.00 mL). The reaction mixture was hydrogenolyzed for 12 h under H₂ gas atmosphere. The reaction mixture was filtered, and the filtrate was evaporated in vacuo. The residue was purified by reverse-phase column chromatography (LiChroprep® RP-18) to give α(2→9) GD2 4 (55 mg, 0.05 mmol, 45%). ¹H NMR (600 MHz, CDCl₃) δ 4.67 (d, 1H, J=8.5 Hz), 4.52 (d, 1H, J=7.9 Hz), 4.48 (d, 1H, J=8.0 Hz), 4.14 (dd, 1H, J=9.8, 2.8 Hz), 4.10 (d, 1H, J=2.8 Hz), 3.99 (d, 1H, J=1.7 Hz), 3.97-3.92 (m, 10H), 3.91 (d, 1H, J=2.2 Hz), 3.89-3.58 (m, 16H), 3.56 (dd, 1H, J=9.3, 1.3 Hz), 3.47 (d, 1H, J=10.3 Hz), 3.36 (t, 1H, J=12.1 Hz), 3.29 (t, 1H, J=8.5 Hz), 2.99 (t, 2H, J=7.5 Hz), 2.73 (dd, 1H, J=12.3, 4.5 Hz), 2.67 (dd, 1H, J=12.4, 4.5 Hz), 2.04 (s, 3H), 2.03 (s, 3H), 2.01 (s, 3H), 1.93 (t, 2H, J=12.1 Hz), 1.71-1.64 (m, 4H), 1.48-1.43 (m, 2H); ¹³C NMR (150 MHz, CDCl₃) δ 174.94, 174.90, 174.8, 174.1, 173.6, 102.7, 102.5, 102.0, 101.5, 100.1, 78.7, 78.6, 77.1, 74.8, 74.4, 74.3, 74.1, 72.9, 72.7, 72.4, 71.7, 71.2, 70.8, 70.5, 70.0, 68.9, 68.3, 67.8, 64.8, 62.6, 61.1, 60.6, 52.2, 51.9, 51.5, 48.8, 40.1, 39.3, 36.9, 28.1, 26.5, 26.3, 23.2, 22.5, 22.1, 22.0, 21.99; HRMS (ESI-TOF) Calcd for C₄₇H₈₀N₄O₃₂ [M−H]⁻ 1213.4834, found 1213.4830.

### Example 1 Evaluation of Subject Having Pre-HD or HD

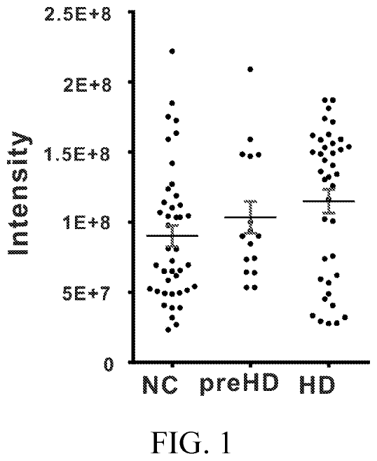

The use of the synthetic ganglio-oligosaccharides in evaluating the occurrence or the likelihood of occurrence of HD was examined in this example. The result were respectively depicted in FIG. 1 and Tables 1-3.

### 1.1 The Difference of Anti-Glycan Antibody Levels in Normal Control, Pre-HD, and HD Patients' Plasma

To start the proposed examination using this focused glycan array, the synthesized mammalian gangliosides were used to prepare glycan array by using an array spotter to spot atto-mole of 28 different kinds of aforementioned chemically synthesized glycans focusing on the glycan moiety of gangliosides (Table 1). The array was designed to have more than 10 repeating spots for each glycan. Meanwhile, the plasma samples were collected from normal controls (NC) (n=42), pre-HID cases (n=16), and HD cases (n=39) (Table 2). The amount of glycans used was in atto-mole quantity. The case numbers, gender distribution, and the clinical data are shown in Table 2. One μl of human plasma was diluted to 100 fold then applied onto the array after blocking. The fluorescent labeled secondary antibodies for IgG or IgM were employed to display binding signal.

TABLE 1
The number, name, and structure of synthetic GSLs glycans for microarray construction

| Compound | |
|---|---|
| (Name) | Structure |
| G1 ((2,3Gal)D) | 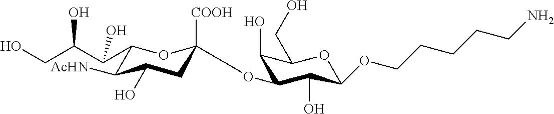 |
| G2 (GM3) | 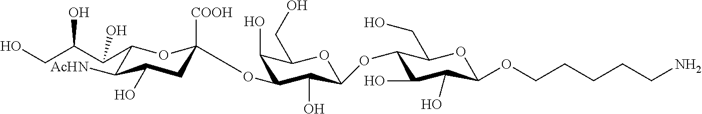 |
| G3 (SLacNAc6SO₃) | 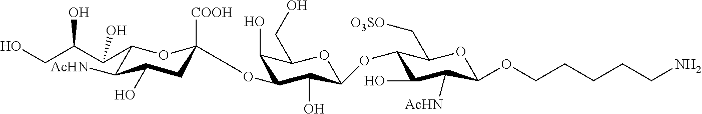 |
| G4 (SLe^{x}) | 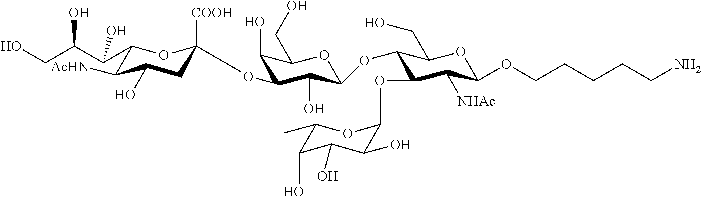 |
| G5 (SiaGalGalNAc) | 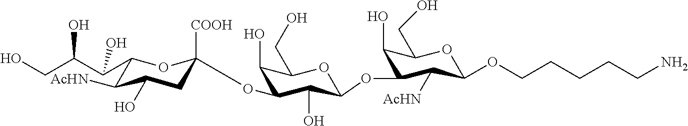 |
| G6 | 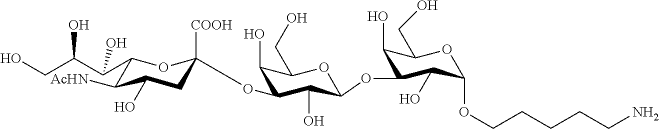 |
| G7 | 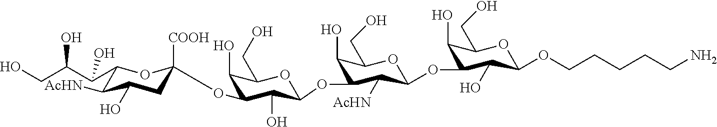 |
| G8 (SSEA-4) | 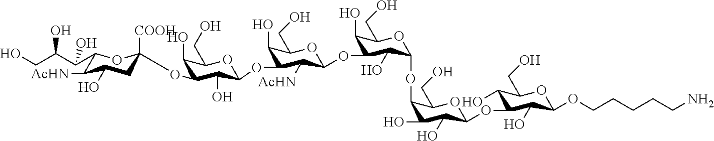 |
| G9 (DSGG) | 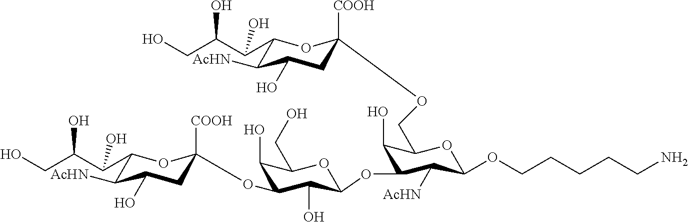 |
| G10 (GM2) | 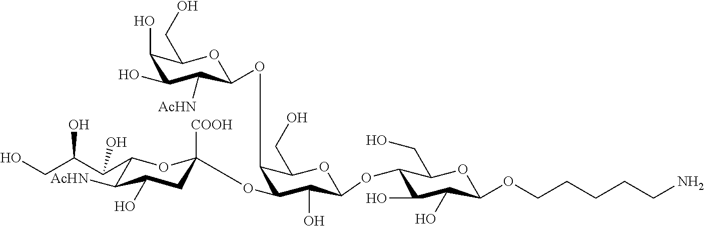 |
| G11 (GM1) | 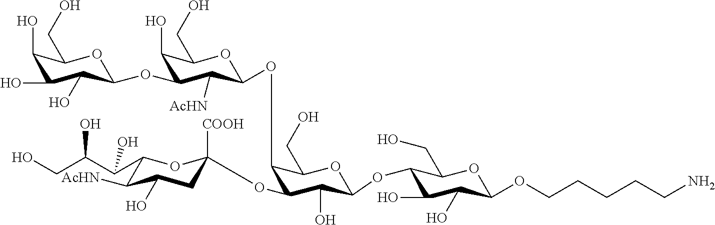 |
| G12 (Fucosyl GM1) | 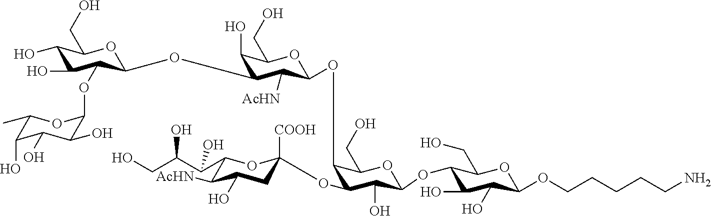 |
| G13 (Fucosyl GM3) | 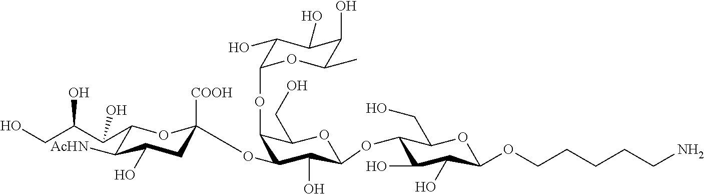 |
| G14 | 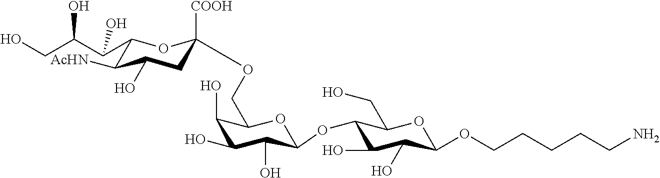 |
| G15 | 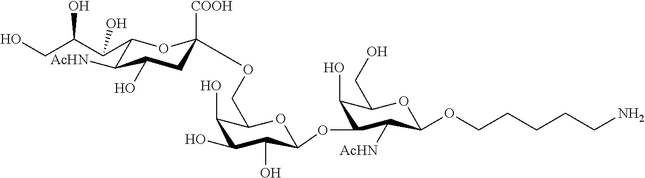 |
| G16 | 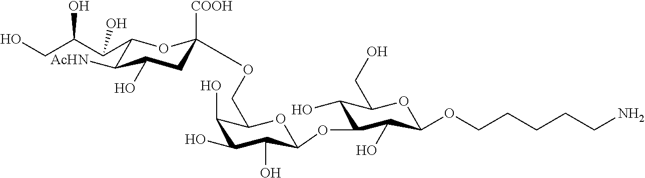 |
| G17 | 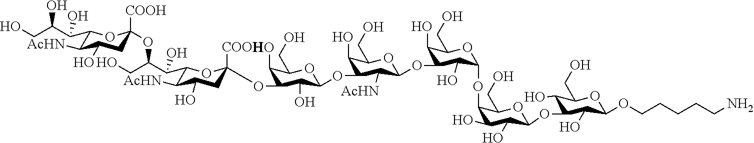 |
| G18 (GD2) | 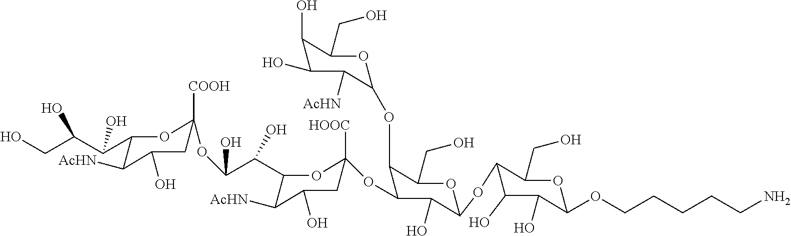 [SMILES: CC(=O)NC1C(OC2C(CO)OC(OC3C(CO)OC(OCCCCCN)C(O)C3O)C(O)C2OC2(C(=O)O)CC(O)C(NC(C)=O)C([C@@H](O)[C@@H](O)OC3(C(=O)O)CC(O)C(NC(C)=O)C([C@H](O)[C@H](O)CO)O3)O2)OC(CO)C(O)C1O] |
| G19 (GD3) | 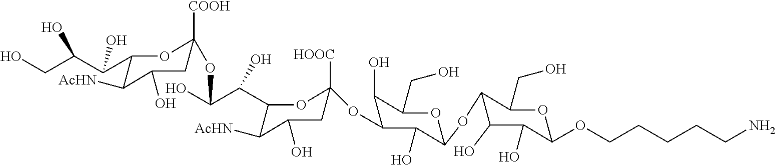 [SMILES: CC(=O)NC1C(O)CC(OC2C(O)C(CO)OC(OC3C(CO)OC(OCCCCCN)C(O)C3O)C2O)(C(=O)O)OC1[C@@H](O)[C@H](O)OC1(C(=O)O)CC(O)C(NC(C)=O)C([C@H](O)[C@H](O)CO)O1] |
| G20 (GD1b) | 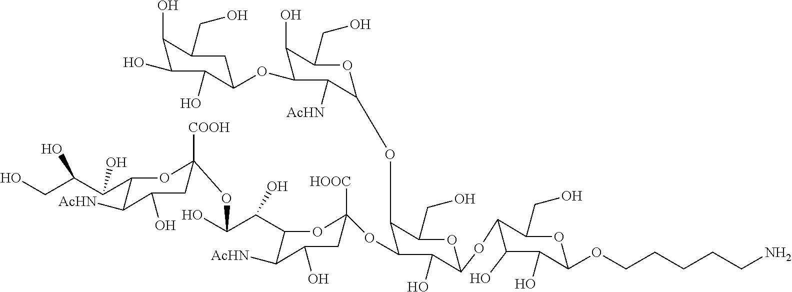 [SMILES: CC(=O)NC1C(OC2C(CO)OC(OC3C(CO)OC(OCCCCCN)C(O)C3O)C(O)C2OC2(C(=O)O)CC(O)C(NC(C)=O)C([C@@H](O)[C@H](O)OC3(C(=O)O)CC(O)C(NC(C)=O)C([C@H](O)[C@H](O)CO)O3)O2)OC(CO)C(O)C1OC1CC(CO)C(O)C(O)C1O] |
| G21 | 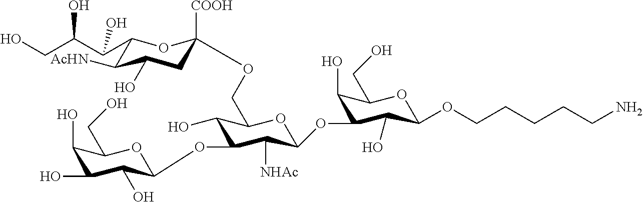 |
| G22 | 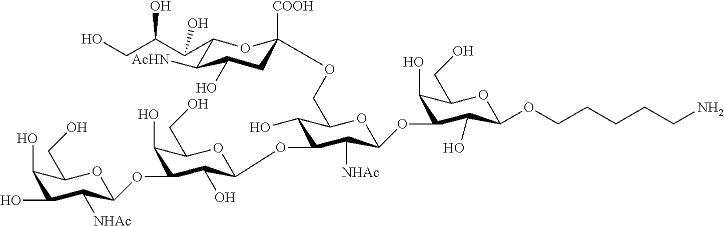 |
| G23 | 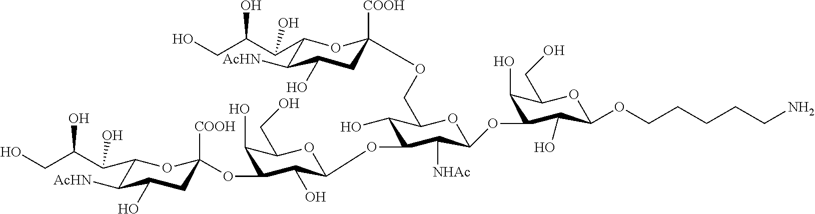 |
| G24 | 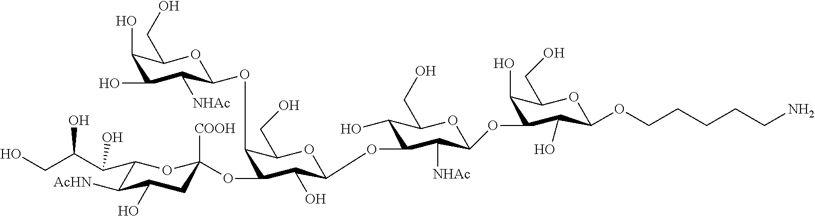 |
| G25 (2,9_GD2) | 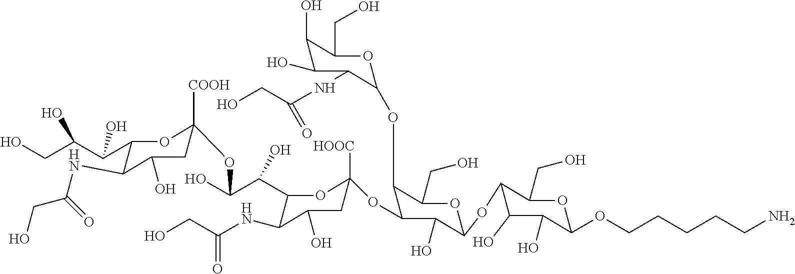 [SMILES: NCCCCCOC1OC(CO)C(OC2OC(CO)C(OC3OC(CO)C(O)C(O)C3NC(=O)CO)C(OC3(C(=O)O)CC(O)C(NC(=O)CO)C([C@@H](O)[C@H](O)OC4(C(=O)O)CC(O)C(NC(=O)CO)C([C@H](O)[C@H](O)CO)O4)O3)C2O)C(O)C1O] |
| G26 (GD2-3GC) | 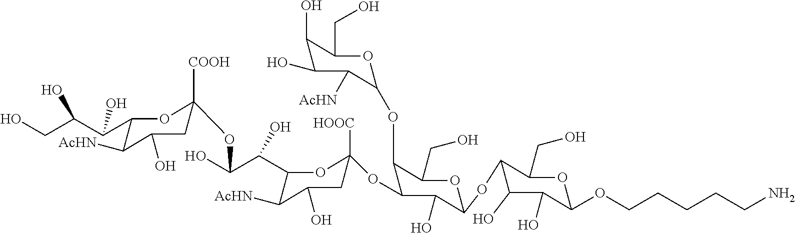 [SMILES: CC(=O)NC1C(OC2C(CO)OC(OC3C(CO)OC(OCCCCCN)C(O)C3O)C(O)C2OC2(C(=O)O)CC(O)C(NC(C)=O)C([C@@H](O)[C@H](O)OC3(C(=O)O)CC(O)C(NC(C)=O)C([C@H](O)[C@H](O)CO)O3)O2)OC(CO)C(O)C1O] |
| G27 | 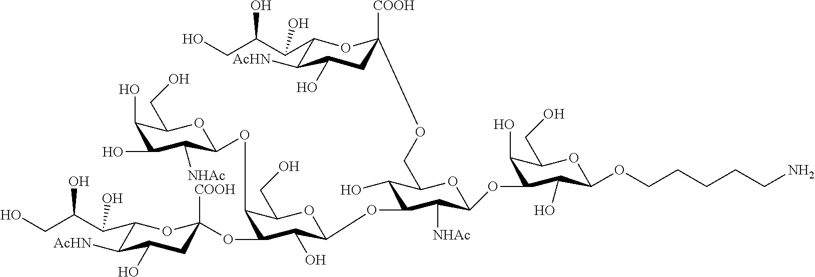 |
| G28 (GD1a) | 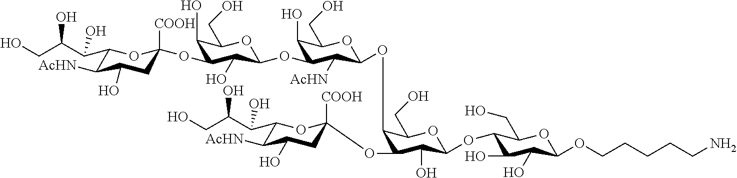 |

* Non-biological glycans do not have names

TABLE 2
Demographic characteristics of the normal controls, pre-HD, and HD

| | Controls | pre-HD | HD | |
|---|---|---|---|---|
| | (n = 42) | (n = 16) | (n = 39) | P value |
| Female | 20 (47.6%) | 8 (50%) | 15 (38.5%) | 0.6260^{a} |
| Male | 22 (52.4%) | 8 (50%) | 24 (61.5%) | |
| Age (Mean ± SD) | 44.7 ± 1.8 | 34.75 ± 3.57 | 46.74 ± 1.82 | 0.0044^{b} |
| Age at onset (Mean ± SD) | | | 41.97 ± 1.76 | |
| Expanded CAG repeat number | | 42.38 ± 0.90 | 45.15 ± 0.78 | 0.0436^{c} |
| (Mean ± SD) | | | | |
| HD duration (Mean ± SD) | | | 4.64 ± 0.56 | |
| Motor score (Mean ± SD) | | 0 | 25.05 ± 3.37 | |
| Independence scale (Mean ± SD) | | 100 | 82.89 ± 3.15 | |
| Functional capacity | | 13 | 10.16 ± 0.55 | |
| (Mean ± SD) | | | | |

^{a}Chi-square test
^{b}F-test
^{c}unpaired Student's T-test
HD: Huntington's Disease; PreHD: Pre-manifest HD carriers; UHDRS: The Unified Huntington's Disease Rating Scale. Scale ranges (normal to most severe) include motor score (0 to 124), independence scale (100 to 10), and functional capacity (13 to 0).

The results indicated that only auto-IgM, but not auto-IgG, antibodies showed differences between the controls and the diseased samples, probably due to low immunogenicity of glycan in human body. Therefore, the natural IgM antibodies against the glycan spotted were examined. Different anti-glycan IgM levels among plasma of NC, pre-HD, and HD patients were compared (data not shown). Interestingly, a significant difference in IgM antibody level in pre-HD cases that have not shown any clinical symptom was found. Specifically, the anti-glycan IgM levels were elevated in pre-HD cases and low in both NC and HD patients, suggesting that the IgM levels of pre-HD offers a measurable abnormality other than the genetic factor. Moreover, the IgMs that exhibited differences in pre-HD and HD groups may be potential biomarkers to indicate the transition and severity from pre-HD to HD stage. Since most auto-glycan IgM antibodies in pre-HD cases detected in the glycan array were higher, whether the phenomenon is attributed by higher total IgM level in pre-HD cases was examined. Therefore, equal amount of plasma from each cohort was subjected to slot-blot and detected total IgM levels by anti-IgM secondary antibody. The results showed no difference among NC, pre-HD cases, and HD patients, demonstrating that the signal changes in the glycan array are specific for auto-glycan antibody (FIG. 1).

### 1.2 The Correlation of Auto-Glycan Antibody Level and Age in Normal Control

To understand the relationship between each auto-glycan antibody and subject age, the pairwise analysis of Pearson's correlation was performed between the 28 auto-glycan antibodies and age in normal controls. Each glycan were assigned for a glycan number (G1 to G28) to facilitate statistical analysis. The result showed that most auto-glycan antibodies did no correlate with subjects' age, except for G12 (fucosyl GM1) and G18 (GD2) (data not shown). Interestingly, many auto-glycan antibodies showed significant positive correlation to each other. The glycan G1 displayed the highly positive correlation with G2 to G28, except for G13 (p=0.1397) (data not shown). However, G13, G20, G22, and G27 exhibited moderate or low correlation with other auto-glycan antibody signals (data not shown). We proposed that the correlation might be attributed from similarity among glycan structures.

### 1.3 Logistic Regression Analysis of Total 28 Glycan to Search for the Potential Biomarkers

Next, univariate analysis of the glycan array signal to identify potential biomarkers was performed. Logistic regression analysis was first used to identify the candidates. Three comparisons were made including (1) NC v.s. pre-HD cases; (2) pre-HD cases v.s. HD patients; and (3) NC v.s. HD patients. The results were listed in Table 3. The significant level of this analysis took multiple comparison into consideration, in which the Bonferroni corrected p-value (0.05/28=about 0.0018) was used. In the analysis for NC vs. pre-HD, most glycan signals were higher in pre-HD patients than that in controls, which corresponded to an odds ratio>1. Among them, the p-values for G8, G17, and G20 signals were lower than 0.0018. In the analysis for pre-HD vs. HD, all odds ratios were <1. The p-values for G8, G9, G11, G14, G18, G20, G23, and G27 were also lower than 0.0018. In the analysis for NC vs. HD, the p-values for G1, G4, and G5 were lower than 0.0018, where all odds ratios for these 3 glycans were <1.

TABLE 3
Screening for candidate glycan biomarkers for NC vs. pre-HD, pre-HD vs. HD,
and NC vs. HD using univariate logistic regression analyses

| | | | | | | | NC vs HD | | |
|---|---|---|---|---|---|---|---|---|---|
| Glycan | NC vs pre-HD | | | pre-HD vs HD | | | | | P |
| No. | OR | 95% CI | P value | OR | 95% CI | P value | OR | 95% CI | value |
| G1 | 1.04 | 1.01-1.06 | 0.0061 | 0.92 | 0.87-0.98 | 0.0049 | 0.91 | 0.86-0.96 | 0.0002 |
| G2 | 1.03 | 1.01-1.05 | 0.0078 | 0.97 | 0.95-0.99 | 0.0047 | 0.98 | 0.95-1.00 | 0.0967 |
| G3 | 1.02 | 1.00-1.04 | 0.0174 | 0.97 | 0.95-0.99 | 0.0118 | 0.98 | 0.96-1.00 | 0.0646 |
| G4 | 1.04 | 1.00-1.07 | 0.0329 | 0.91 | 0.85-0.97 | 0.0053 | 0.91 | 0.86-0.97 | 0.0017 |
| G5 | 1.03 | 1.01-1.06 | 0.0025 | 0.95 | 0.91-0.99 | 0.0068 | 0.95 | 0.91-0.98 | 0.0009 |
| G6 | 1.02 | 1.00-1.03 | 0.0079 | 0.99 | 0.97-1.00 | 0.0056 | 1.00 | 0.99-1.01 | 0.9321 |
| G7 | 1.02 | 1.01-1.03 | 0.006 | 0.99 | 0.98-1.00 | 0.0028 | 1.00 | 0.99-1.02 | 0.6224 |
| G8 | 1.02 | 1.01-1.03 | 0.0016 | 0.99 | 0.98-1.00 | 0.0016 | 1.00 | 0.99-1.01 | 0.8576 |
| G9 | 1.04 | 1.01-1.07 | 0.0035 | 0.89 | 0.84-0.95 | 0.0007 | 0.92 | 0.86-0.97 | 0.0032 |
| G10 | 1.06 | 1.02-1.10 | 0.0052 | 0.91 | 0.86-0.97 | 0.0021 | 0.94 | 0.90-0.99 | 0.0218 |
| G11 | 1.03 | 1.01-1.06 | 0.0097 | 0.99 | 0.98-0.99 | 0.0006 | 1.01 | 1.00-1.02 | 0.1571 |
| G12 | 1.06 | 1.02-1.10 | 0.006 | 0.97 | 0.95-0.99 | 0.0045 | 1.03 | 1.00-1.06 | 0.0456 |
| G13 | 1.02 | 1.01-1.03 | 0.0055 | 0.99 | 0.98-1.00 | 0.0063 | 1.01 | 1.00-1.02 | 0.089 |
| G14 | 1.05 | 1.01-1.08 | 0.0134 | 0.92 | 0.88-0.97 | 0.0011 | 0.95 | 0.91-1.00 | 0.0115 |
| G15 | 1.01 | 1.00-1.02 | 0.0038 | 0.99 | 0.98-1.00 | 0.0034 | 1.00 | 0.99-1.01 | 0.7193 |
| G16 | 1.01 | 1.00-1.03 | 0.1107 | 0.98 | 0.97-1.00 | 0.0685 | 0.99 | 0.97-1.01 | 0.1367 |
| G17 | 1.01 | 1.01-1.02 | 0.0018 | 0.99 | 0.99-1.00 | 0.0081 | 1.00 | 1.00-1.01 | 0.6321 |
| G18 | 1.03 | 1.01-1.05 | 0.0171 | 0.94 | 0.91-0.98 | 0.0012 | 0.97 | 0.94-0.99 | 0.0161 |
| G19 | 1.02 | 1.00-1.03 | 0.0425 | 0.98 | 0.96-1.00 | 0.0141 | 0.98 | 0.96-1.00 | 0.0353 |
| G20 | 1.01 | 1.01-1.02 | 0.0009 | 0.99 | 0.99-1.00 | 0.0005 | 1.00 | 1.00-1.01 | 0.4044 |
| G21 | 1.02 | 1.01-1.04 | 0.0059 | 0.98 | 0.96-0.99 | 0.0051 | 0.98 | 0.95-1.00 | 0.05 |
| G22 | 1.01 | 1.00-1.012 | 0.0118 | 0.99 | 0.99-1.00 | 0.0089 | 1.00 | 0.99-1.01 | 0.8122 |
| G23 | 1.05 | 1.01-1.10 | 0.0155 | 0.90 | 0.84-0.96 | 0.0011 | 0.94 | 0.89-0.99 | 0.0195 |
| G24 | 1.05 | 1.01-1.09 | 0.0084 | 0.97 | 0.95-1.00 | 0.0142 | 1.01 | 0.98-1.03 | 0.6523 |
| G25 | 1.02 | 1.00-1.04 | 0.0275 | 0.94 | 0.89-0.98 | 0.0051 | 0.97 | 0.94-1.00 | 0.0503 |
| G26 | 1.03 | 1.01-1.05 | 0.0088 | 0.97 | 0.95-0.99 | 0.0069 | 0.99 | 0.97-1.01 | 0.4405 |
| G27 | 1.04 | 1.01-1.07 | 0.0113 | 0.88 | 0.81-0.95 | 0.0018 | 0.94 | 0.89-0.99 | 0.019 |
| G28 | 1.02 | 1.01-1.04 | 0.0096 | 0.98 | 0.97-0.99 | 0.0044 | 1.00 | 0.98-1.02 | 0.9496 |

Next, multiple logistic regression analysis for selecting independent factors was employed so as to discriminate HD status. Age is included in the analysis for three groups. Since repeat number was not available in NC cases, repeat number was only placed in group 2 (pre-HID vs HD). All glycan signals that were significant in Bonferroni analysis (p<0.0018) were included. For group 1 (NC vs pre-HD), Age, G8, G17, G20 were included. These glycan were all significant in Bonferroni analysis. For Group 2, Age, repeat number, G8, G9, G11, G14, G18, G20, G23, G27 were included. There glycan were all p<0.0018 in Bonferroni analysis. For group 3, age, G4, G5 were included. Three methods were used including stepwise, forward, and backward selection approaches. The models with the minimum AIC values among the three methods were considered the one with the best goodness of fit. The analysis indicated that the backward method was the most suitable methods for three groups (data not shown). In the NC and pre-HD group, it was found that G17 (p<0.0351) and G20 (GD1b) (p<0.0472) were the potential biomarker candidates (data not shown). In the pre-HD versus HD group, three candidates including age (p<0.0083), repeat number (p<0.0295), and G20 (GD1b) (p<0.0057) were found, whereas, in the NC and HD group, only G5 (SiaGalGalNAc) possessed the potential (p<0.0009) (data not shown).

### 1.4 The Combination of Potential Glycan Markers Improves the AUC for Clinical Diagnosis

To further evaluate the clinical performance of independent factors that were selected in the multivariate logistic regression analysis, receiver operating characteristic curve (ROC) analysis with the area under ROC curve (AUC) was performed. The sensitivity and specificity of the selected anti-glycan antibody as plasma biomarkers for HD were calculated. When comparing pre-HD cases vs. HD patients, the AUC for age, repeat number, and G20 were 0.79 (95% CI, 0.62-0.95), 0.74 (95% CI, 0.68-1), and 0.81 (95% CI, 0.65-0.97), respectively (data 20) not shown). Combining age and repeat number improved the AUC to 0.86 (95% CI, 0.74-0.99; data not shown). Furthermore, when the novel biomarker G20 (GD1b) was further combined, the AUC score could reach 0.95 (95% CI, 0.85-1; data not shown). This combination indicated the best discrimination capacity to differentiate NC and pre-HD group. NC vs. pre-HD cases were also analyzed with G17, G20, and G17 plus G20. The AUCs for G17 and G20 were 0.79 (95% CI, 0.62-0.97) and 0.83 (95% CI, 0.68-0.99), respectively (data not shown). The combination of G17 and G20 gave small improvement in AUC to 0.84 (95% CI, 0.68-1; data not shown). However the differences were not statistically significant when comparing to the AUC of G17 or G20 only. In the analysis of NC vs. HD, the AUC for G5 is 0.72 (95% CI, 0.31-0.83; data not shown). Overall, the results discovered and demonstrated a novel biomarker G20 (GD1b) to differentiate NC and pre-HD group.

In conclusion, the data of the present example indicated significant differences in auto-glycan antibodies among the plasma of NC cases, pre-HD cases, and HD patients. Notably, most of the altered auto-glycan antibodies tend to increase in pre-HD cases and decrease in HD patients, whereas the increasing trend remains for anti-fucosyl GMI and anti-fucosyl GM3 from NC cases to HD patients. This may indicate abnormal level of fucosylation in HD. Through statistical analysis, to the data demonstrated the AUC analysis differentiating pre-HD and HD cases increased from 0.86 to 0.95 with addition of anti-GD1b antibody response to age and CAG repeat number.

### Example 2 Evaluation of Subject Having MCI or AD

In addition to HD, the use of the synthetic ganglio-oligosaccharides in evaluating the occurrence or the likelihood of occurrence of AD was also investigated in this experiment. The result were respectively depicted in Tables 4-5.

### 2.1 The Difference of Anti-Glycan Antibody Levels in Normal Control, MCI, and AD Patients' Plasma

To start the examination, normal control, MCI and AD patient plasma were collected for the focused glycan array. The sample number used were 23 normal control, 67 MCI, and 54 patent samples for AD. The focused glycan arrays were prepared by array spotter to spot 28 chemically synthesized glycan portions of glycans (Table 1). The amount of glycans used was in atto-mole quantity. The array was designed to have at least 10 repeating spots for each glycan and 1 μl of human plasma was diluted to 100 fold and applied onto the array after blocking as described in the method and the subsequent procedure were described in the method. The fluorescent labeled secondary antibody for IgM was employed to show the binding signal.

The change of anti-glycan IgM levels in the plasma of NC, MCI, and AD patients were detected (data not shown). The result indicated that high levels of anti-glycan antibody were in the MCI group compared with normal control and AD patients (data not shown). The most part of anti-glycan antibody levels increased in MCI stage and decrease in AD (data not shown). Further, the level change of normal control and AD patient was analyzed. Interestingly, only the level of anti-GM2 antibody was significantly decreased in AD patients compared with that of normal control (data not shown). The others were presented high level in the AD patients' plasma. The intensity of total 28 anti-glycan antibody levels was listed in Table 4. Since the difference of intensity was too large that could affect the judgement of the significance, Mann-Whitney U test was used to present the result. The result indicated that all glycan signals in the group of NC v.s. MCI were significant except for G10 (p=0.2638). Also, all glycan signals in MCI v.s. AD were significantly different except for G13 (p=0.1184). However, in the group of NC v.s. AD, only some signals were significantly different, including G2 (p=0.0446), G4 (p=0.0446), G8 (p=0.0446), G10 (p<0.001), G12 (p=0.016), G16 (0.0472), G19 (p=0.0295), G21 (p=0.0449), G24 (p=0.0255), and G27 (p=0.0024).

TABLE 4
The univariate analysis of the intensity of anti-glycan antibody

| | | | | | P value | | |
|---|---|---|---|---|---|---|---|
| Glycan | | NC | MCI | AD | NC vs | MCI vs | NC vs |
| No. | | N = 22 | N = 67 | N = 53 | MCI | AD | AD |
| G1 | Mean | 81.0 | 126.4 | 82.8 | 0.0467 | 0.0137 | 0.9188 |
| | (SD) | (78.3) | (121.7) | (66.0) | | | |
| | Median | 65.3 | 78.8 | 61.4 | 0.0052 | 0.0011 | 0.8432 |
| | (IQR) | (46.2-80.1) | (62.9-156.4) | (49.5-88.7) | | | |
| G2 | Mean | 57.0 | 116.0 | 107.5 | 0.002 | 0.7558 | 0.02 |
| | (SD) | (23.7) | (145.1) | (149.2) | | | |
| | Median | 52.0 | 76.0 | 65.2 | 0.0002 | 0.0452 | 0.0466 |
| | (IQR) | (39.5-64.5) | (57.1-107.6) | (47.3-97.4) | | | |
| G3 | Mean | 59.7 | 225.9 | 100.2 | 0.0053 | 0.0398 | 0.0311 |
| | (SD) | (21.6) | (470.2) | (129.2) | | | |
| | Median | 56.5 | 118.5 | 61.9 | <.0001 | <.0001 | 0.2766 |
| | (IQR) | (45.7-66.3) | (71.0-192.3) | (47.8-97.2) | | | |
| G4 | Mean | 46.1 | 81.0 | 68.2 | <.0001 | 0.28 | 0.0575 |
| | (SD) | (17.2) | (39.1) | (78.4) | | | |
| | Median | 38.3 | 71.8 | 51.5 | <.0001 | <.0001 | 0.0466 |
| | (IQR) | (34.9-53.2) | (54.7-102.1) | (38.8-61.4) | | | |
| G5 | Mean | 77.8 | 168.6 | 128.5 | 0.0067 | 0.3307 | 0.1695 |
| | (SD) | (87.7) | (219.0) | (228.7) | | | |
| | Median | 57.7 | 110.5 | 71.2 | <.0001 | 0.0002 | 0.1122 |
| | (IQR) | (47.5-75.8) | (71.2-208.4) | (52.2-102.8) | | | |
| G6 | Mean | 114.3 | 307.1 | 196.3 | 0.0001 | 0.0738 | 0.1178 |
| | (SD) | (115.5) | (336.4) | (331.5) | | | |
| | Median | 73.9 | 192.9 | 101.8 | 0.0001 | 0.0002 | 0.1827 |
| | (IQR) | (54.1-120.6) | (97.2-381.2) | (70.1-137.4) | | | |
| G7 | Mean | 118.7 | 262.4 | 173.4 | 0.0002 | 0.0554 | 0.1942 |
| | (SD) | (101.3) | (242.2) | (260.0) | | | |
| | Median | 83.7 | 175.7 | 98.9 | 0.0001 | 0.0002 | 0.364 |
| | (IQR) | (55.7-129.0) | (115.0-352.9) | (59.3-168.6) | | | |
| G8 | Mean | 197.6 | 573.2 | 367.7 | 0.0015 | 0.216 | 0.2927 |
| | (SD) | (356.0) | (695.4) | (1028.7) | | | |
| | Median | 107.3 | 291.6 | 165.5 | 0.0002 | 0.0032 | 0.0466 |
| | (IQR) | (60.7-166.3) | (128.3-731.9) | (89.6-231.0) | | | |
| G9 | Mean | 46.6 | 91.0 | 51.4 | <.0001 | <.0001 | 0.3903 |
| | (SD) | (23.4) | (68.8) | (20.8) | | | |
| | Median | 38.9 | 71.8 | 53.8 | <.0001 | <.0001 | 0.1008 |
| | (IQR) | (35.2-45.0) | (54.9-92.0) | (43.1-79.9) | | | |
| G10 | Mean | 79.2 | 97.3 | 46.3 | 0.0644 | <.0001 | 0.0001 |
| | (SD) | (32.4) | (55.1) | (13.4) | | | |
| | Median | 66.1 | 59.5 | 42.6 | 0.2638 | <.0001 | <.0001 |
| | (IQR) | (56.4-98.2) | (50.6-76.5) | (32.6-52.6) | | | |
| G11 | Mean | 191.4 | 624.8 | 122.4 | 0.0118 | 0.0029 | 0.1425 |
| | (SD) | (204.3) | (1327.3) | (97.6) | | | |
| | Median | 112.1 | 248.2 | 64.2 | 0.0013 | <.0001 | 0.0763 |
| | (IQR) | (79.4-209.6) | (150.4-548.5) | (64.2-139.7) | | | |
| G12 | Mean | 57.3 | 168.7 | 89.8 | <.0001 | 0.0007 | 0.0093 |
| | (SD) | (26.4) | (163.2) | (78.3) | | | |
| | Median | 49.5 | 101.7 | 59.5 | <.0001 | <.0001 | 0.016 |
| | (IQR) | (36.5-74.9) | (66.5-245.0) | (48.7-108.9) | | | |
| G13 | Mean | 86.8 | 159.1 | 141.3 | 0.0053 | 0.5384 | 0.0349 |
| | (SD) | (71.4) | (164.4) | (147.5) | | | |
| | Median | 68.7 | 105.5 | 77.0 | 0.0011 | 0.1184 | 0.0513 |
| | (IQR) | (48.0-87.0) | (67.1-172.1) | (62.1-154.4) | | | |
| G14 | Mean | 57.7 | 130.6 | 71.1 | <.0001 | <.0001 | 0.1388 |
| | (SD) | (30.1) | (107.3) | (45.2) | | | |
| | Median | 47.6 | 88.4 | 52.8 | <.0001 | <.0001 | 0.2817 |
| | (IQR) | (40.2-67.9) | (63.0-158.3) | (42.0-76.2) | | | |
| G15 | Mean | 116.5 | 269.9 | 183.4 | <.0001 | 0.069 | 0.0804 |
| | (SD) | (80.4) | (265.3) | (244.7) | | | |
| | Median | 92.8 | 176.8 | 91.9 | 0.0004 | 0.0012 | 0.2307 |
| | (IQR) | (56.5-149.1) | (107.4-325.1) | (73.7-191.0) | | | |
| G16 | Mean | 68.9 | 190.8 | 106.9 | <.0001 | 0.0035 | 0.0219 |
| | (SD) | (34.7) | (197.3) | (105.0) | | | |
| | Median | 55.1 | 105.5 | 68.8 | <.0001 | 0.0005 | 0.0472 |
| | (IQR) | (42.0-83.3) | (69.3-242.9) | (53.8-129.9) | | | |
| G17 | Mean | 215.8 | 831.6 | 555.1 | 0.0006 | 0.3905 | 0.2557 |
| | (SD) | (401.3) | (1225.5) | (2060.8) | | | |
| | Median | 105.3 | 341.3 | 173.1 | 0.0007 | 0.0107 | 0.0527 |
| | (IQR) | (52.9-175.7) | (104.5-1123.6) | (97.0-260.8) | | | |
| G18 | Mean | 56.1 | 123.5 | 67.9 | 0.0003 | 0.0025 | 0.1562 |
| | (SD) | (26.4) | (137.2) | (44.1) | | | |
| | Median | 43.9 | 88.0 | 55.9 | <.0001 | <.0001 | 0.1626 |
| | (IQR) | (38.3-68.9) | (58.9-146.1) | (43.2-72.1) | | | |
| G19 | Mean | 53.2 | 98.3 | 79.0 | <.0001 | 0.1234 | 0.0063 |
| | (SD) | (21.0) | (74.1) | (58.4) | | | |
| | Median | 41.5 | 77.9 | 58.1 | <.0001 | 0.0039 | 0.0295 |
| | (IQR) | (37.1-70.8) | (56.7-104.1) | (46.0-86.0) | | | |
| G20 | Mean | 234.7 | 744.9 | 246.0 | 0.0223 | 0.0241 | 0.8496 |
| | (SD) | (210.0) | (1749.8) | (243.9) | | | |
| | Median | 160.5 | 302.2 | 139.3 | 0.0181 | 0.0027 | 0.9074 |
| | (IQR) | (85.0-339.4) | (137.4-634.7) | (87.4-379.5) | | | |
| G21 | Mean | 47.4 | 118.7 | 59.6 | <.0001 | <.0001 | 0.0285 |
| | (SD) | (14.4) | (78.9) | (32.5) | | | |
| | Median | 41.4 | 88.2 | 50.5 | <.0001 | <.0001 | 0.0499 |
| | (IQR) | (37.7-61.6) | (63.4-157.6) | (41.5-61.3) | | | |
| G22 | Mean | 126.4 | 359.6 | 202.6 | 0.0003 | 0.0195 | 0.0449 |
| | (SD) | (99.1) | (479.2) | (223.9) | | | |
| | Median | 87.7 | 218.2 | 124.7 | 0.0002 | 0.0015 | 0.1122 |
| | (IQR) | (71.2-139.7) | (115.3-401.3) | (75.7-212.9) | | | |
| G23 | Mean | 43.9 | 87.6 | 54.3 | <.0001 | <.0001 | 0.0281 |
| | (SD) | (10.7) | (55.4) | (29.6) | | | |
| | Median | 39.7 | 69.5 | 43.6 | <.0001 | <.0001 | 0.1441 |
| | (IQR) | (37.6-47.2) | (54.1-94.2) | (37.5-59.7) | | | |
| G24 | Mean | 45.9 | 134.4 | 60.4 | <.0001 | <.0001 | 0.0233 |
| | (SD) | (14.4) | (132.3) | (39.5) | | | |
| | Median | 40.4 | 80.7 | 49.0 | <.0001 | <.0001 | 0.0255 |
| | (IQR) | (35.1-58.3) | (65.5-124.3) | (39.5-66.7) | | | |
| G25 | Mean | 56.4 | 144.9 | 75.1 | <.0001 | 0.0001 | 0.0251 |
| | (SD) | (20.8) | (131.8) | (50.1) | | | |
| | Median | 47.8 | 95.7 | 59.0 | <.0001 | <.0001 | 0.0961 |
| | (IQR) | (38.6-71.3) | (69.3-163.6) | (45.7-82.2) | | | |
| G26 | Mean | 88.6 | 188.7 | 112.6 | 0.0001 | 0.0013 | 0.4013 |
| | (SD) | (86.4) | (129.3) | (120.5) | | | |
| | Median | 65.3 | 141.8 | 72.4 | <.0001 | <.0001 | 0.1925 |
| | (IQR) | (47.7-77.1) | (93.3-242.5) | (57.6-107.2) | | | |
| G27 | Mean | 40.5 | 76.7 | 52.5 | <.0001 | <.0001 | 0.0016 |
| | (SD) | (9.5) | (39.8) | (22.4) | | | |
| | Median | 36.3 | 65.3 | 43.8 | <.0001 | <.0001 | 0.0024 |
| | (IQR) | (34.7-47.3) | (53.4-90.6) | (37.5-58.8) | | | |
| G28 | Mean | 74.2 | 254.8 | 136.0 | <.0001 | 0.0067 | 0.0007 |
| | (SD) | (26.3) | (322.9) | (120.1) | | | |
| | Median | 68.4 | 154.6 | 91.5 | <.0001 | 0.0052 | 0.0602 |
| | (IQR) | (57.8-87.9) | (83.8-288.0) | (59.8-153.0) | | | |

### 2.2 The Correlation of Auto-Glycan Antibody Level, MMSE Score, and Age in Normal Control

To ask whether the plasma auto-glycan antibodies as well as age are correlated, Spearson's correlation was used to analyze the signals (data not shown). The results indicated most auto-glycan antibodies have no correlation with age, except for G3 (positive, p=0.0002), G19 (positive, p=0.0295), and G25 (positive, p=0.0425) (data not shown). The correlation between auto-glycan antibody level and MMSE score was also examined, and the result demonstrated that auto-glycan antibody level present no correlation with MMSE score difference, except for G13 (negative, p=0.0192; data not shown). Interestingly, auto-glycan antibody exhibited highly positive correlation to each other, except for G10 (data not shown). the data suggested that the similarity of structure of glycan maybe affect the auto-antibody recognition.

### 2.3 Logistic Regression Analysis of Total 28 Glycan to Search for the Potential Biomarkers

Logistic regression analysis was used to identify the possible candidates of 28 kinds of auto-glycan antibody. The normal control, MCI, and AD patients were separated into three groups: <1> normal control v.s. MCI; <2> MCI v.s. AD patients; <3> normal control v.s. AD patients; the results are shown in Table 5. The multiple comparisons were applied with Bonferroni correction. The difference of glycan candidates in those three groups was observed. In the first group, the p-value of G3, G4, G9, G19, G21, G23, G24, G25, and G27 were lower than 0.0018, odds ratio>1. In the second group, G9, G10, G11, G14, G18, G21, G23, G24, G25, and G27 were lower than 0.0018, odds ratio<1. Finally, only G10 (and G27) exhibited the significant difference and odd ratio<1 in the third group. Further, the potential glycan candidates in each group were combined with two factors, such as age and MMSE score for the selection step. The selection of logistic regression was used to three methods: stepwise, forward, and backward. The lower AIC score presented the suitable method in the selection. The forward method was employed in the first group and the result indicated that age (p=0.0529), MMSE score (p=0.0152), G4 (p=0.1478), and G27 (p=0.0251) were the potential candidates. Interestingly, the three candidates, including MMSE score (p=0.0001), G4 (p=0.0032), and G11 glycan (p=0.0032), were selected in the second group by using the backward method. The three methods were suitable for the third group selection, and the result only exhibited MMSE score (p=0.0252).

TABLE 5
Logistic regression analysis of the glycans from 1 to 28 between the different
groups of NC, MCI, and AD patients

| | NC vs MCI | | | | | | | | |
|---|---|---|---|---|---|---|---|---|---|
| Glycan | | OR (95% | | MCI vs AD | | | NC vs AD | | |
| No. | Crude | CI) | P value | Crude | OR (95% CI) | P value | Crude | OR (95% CI) | P value |
| G1 | 1.01 | 0.998-1.02 | 0.1125 | 0.993 | 0.987-0.999 | 0.0317 | 1 | 0.993-1.008 | 0.9174 |
| G2 | 1.04 | 1.012-1.07 | 0.0056 | 1 | 0.997-1.002 | 0.7543 | 1.016 | 0.997-1.035 | 0.0989 |
| G3 | 1.04 | 1.017-1.07 | 0.0009 | 0.996 | 0.991-1 | 0.0397 | 1.014 | 0.997-1.03 | 0.107 |
| G4 | 1.09 | 1.037-1.14 | 0.0004 | 0.995 | 0.987-1.004 | 0.2701 | 1.024 | 0.994-1.056 | 0.1225 |
| G5 | 1.01 | 1.002-1.03 | 0.0208 | 0.999 | 0.997-1.001 | 0.3488 | 1.002 | 0.997-1.008 | 0.3823 |
| G6 | 1.01 | 1.002-1.01 | 0.0104 | 0.999 | 0.997-1 | 0.0904 | 1.002 | 0.998-1.005 | 0.3184 |
| G7 | 1.01 | 1.002-1.01 | 0.0101 | 0.998 | 0.996-1 | 0.0727 | 1.002 | 0.998-1.006 | 0.3638 |
| G8 | 1.00 | 1-1.004 | 0.0367 | 1 | 0.999-1 | 0.2247 | 1.001 | 0.999-1.002 | 0.5344 |
| G9 | 1.07 | 1.031-1.12 | 0.0005 | 0.957 | 0.937-0.977 | <.0001 | 1.012 | 0.985-1.04 | 0.3907 |
| G10 | 1.01 | 0.997-1.02 | 0.1553 | 0.912 | 0.88-0.946 | <.0001 | 0.93 | 0.897-0.965 | 0.0001 |
| G11 | 1.00 | 1-1.006 | 0.0462 | 0.992 | 0.988-0.996 | <.0001 | 0.997 | 0.993-1 | 0.0809 |
| G12 | 1.04 | 1.011-1.06 | 0.0035 | 0.993 | 0.988-0.998 | 0.0034 | 1.018 | 1-1.035 | 0.0509 |
| G13 | 1.01 | 1-1.02 | 0.049 | 0.999 | 0.997-1.002 | 0.537 | 1.005 | 0.998-1.012 | 0.1324 |
| G14 | 1.04 | 1.01-1.07 | 0.0032 | 0.986 | 0.978-0.994 | 0.0012 | 1.01 | 0.994-1.026 | 0.2179 |
| G15 | 1.01 | 1.00-1.02 | 0.0074 | 0.998 | 0.997-1 | 0.0842 | 1.003 | 0.998-1.008 | 0.2655 |
| G16 | 1.02 | 1.01-1.04 | 0.0069 | 0.996 | 0.992-0.999 | 0.0145 | 1.011 | 0.998-1.024 | 0.0994 |
| G17 | 1.00 | 1-1.003 | 0.0367 | 1 | 1-1 | 0.3878 | 1.001 | 0.999-1.002 | 0.5235 |
| G18 | 1.04 | 1.01-1.06 | 0.0022 | 0.985 | 0.975-0.994 | 0.0014 | 1.01 | 0.993-1.027 | 0.2568 |
| G19 | 1.05 | 1.02-1.08 | 0.0013 | 0.995 | 0.989-1.002 | 0.1363 | 1.02 | 0.998-1.043 | 0.0708 |
| G20 | 1.00 | 1-1.004 | 0.056 | 0.998 | 0.997-1 | 0.0121 | 1 | 0.998-1.002 | 0.8471 |
| G21 | 1.09 | 1.04-1.14 | 0.0002 | 0.972 | 0.958-0.986 | <.0001 | 1.029 | 0.994-1.065 | 0.1107 |
| G22 | 1.01 | 1.00-1.01 | 0.0063 | 0.998 | 0.997-1 | 0.0482 | 1.003 | 0.999-1.008 | 0.1619 |
| G23 | 1.15 | 1.07-1.23 | 0.0001 | 0.972 | 0.956-0.989 | 0.0009 | 1.031 | 0.99-1.074 | 0.1366 |
| G24 | 1.11 | 1.05-1.16 | <.0001 | 0.977 | 0.962-0.991 | 0.0015 | 1.032 | 0.996-1.069 | 0.0807 |
| G25 | 1.06 | 1.03-1.09 | 0.0002 | 0.986 | 0.977-0.995 | 0.0017 | 1.018 | 0.997-1.04 | 0.0975 |
| G26 | 1.01 | 1.00-1.02 | 0.0054 | 0.994 | 0.99-0.998 | 0.0035 | 1.002 | 0.997-1.008 | 0.4086 |
| G27 | 1.17 | 1.09-1.26 | <.0001 | 0.963 | 0.944-0.982 | 0.0002 | 1.063 | 1.006-1.122 | 0.0285 |
| G28 | 1.02 | 1.01-1.04 | 0.0026 | 0.997 | 0.994-1 | 0.025 | 1.013 | 1-1.027 | 0.0474 |

### 2.4 The Combination of Potential Glycan Markers Improves the AUC for Clinical Diagnosis

To evaluate the potential glycan candidates, a receiver operating characteristic curve (ROC) was created to evaluate the sensitivity and specificity of the anti-glycan antibody as plasma biomarkers for AD. The detection of disease progression in AD is the important issue for clinical diagnosis. Although there was no significant difference between the AUC of the normal control v.s. AD patients, in the group of normal control v.s. MCI, it was found the area under the curve (AUC) in age, MMSE score, G4, and G27 to be 0.73 (95% CI, 0.61-0.85), 0.83 (95% CI, 0.74-0.92), 0.87 (95% CI, 0.77-0.96), and 0.92 (95% CI, 0.85-0.98), respectively (data not shown). The combination with those 4 factor improve the AUC to 0.96 (95% CI, 0.92 -0.999; data not shown). In the group of MCI v.s. AD patients, the score of AUC for MMSE score, G10, and G11 are 0.94 (95% CI, 0.897-0.99), 0.898 (95% CI, 0.84-0.95), and 0.82 (95% CI, 0.75-0.90), respectively (data not shown). The AUC for the combination of MMSE score, G10, and G11 showed the best sensitivity and specificity, 0.99 (95% CI, 0.98-1; data not shown). Taken together, these results demonstrated that the profile of the three markers be a molecular biomarker for diagnosing AD progression.

In conclusion, the present disclosure provides several synthetic ganglio-oligosaccharides for predicting the occurrence of neurodegenerative diseases, for example, AD and HD. Based on the analysis data, a practitioner may make an early diagnosis of a neurodegenerative disease, and accordingly, administering to the subject in need thereof (e.g., the subject at high risk of developing a neurodegenerative disease, or the subject in the early stage of a neurodegenerative disease) in time so as to improve the subject's life quality and life span.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of this invention.

1. A compound having a structure of formula (I), 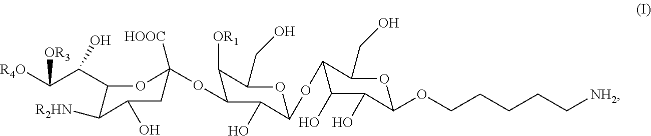
wherein,
R₁ is H, or optionally substituted 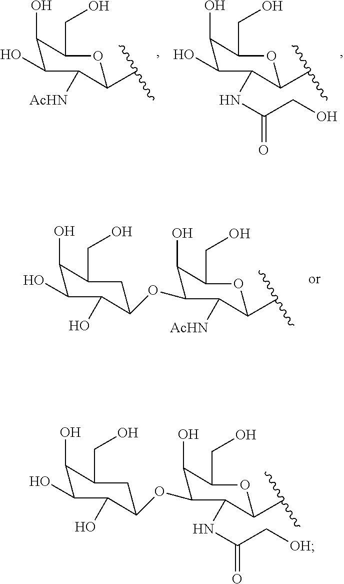 [SMILES: *C1OC(CO)C(O)C(O)C1NC(C)=O ; *C1OC(CO)C(O)C(O)C1NC(=O)CO ; *C1OC(CO)C(O)C(OC2CC(CO)C(O)C(O)C2O)C1NC(C)=O ; *C1OC(CO)C(O)C(OC2CC(CO)C(O)C(O)C2O)C1NC(=O)CO]
R₂ is optionally substituted acetyl or 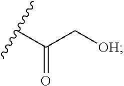 [SMILES: *C(=O)CO]
and
R₃ and Ra are independently H, or optionally substituted

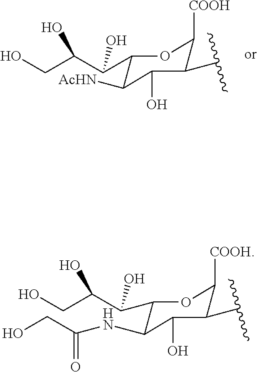 [SMILES: *C1C(C(=O)O)OC([C@H](O)[C@H](O)CO)C(NC(C)=O)C1O ; *C1C(C(=O)O)OC([C@H](O)[C@H](O)CO)C(NC(=O)CO)C1O]

2. The compound of claim 1, wherein the compound is any of the followings,

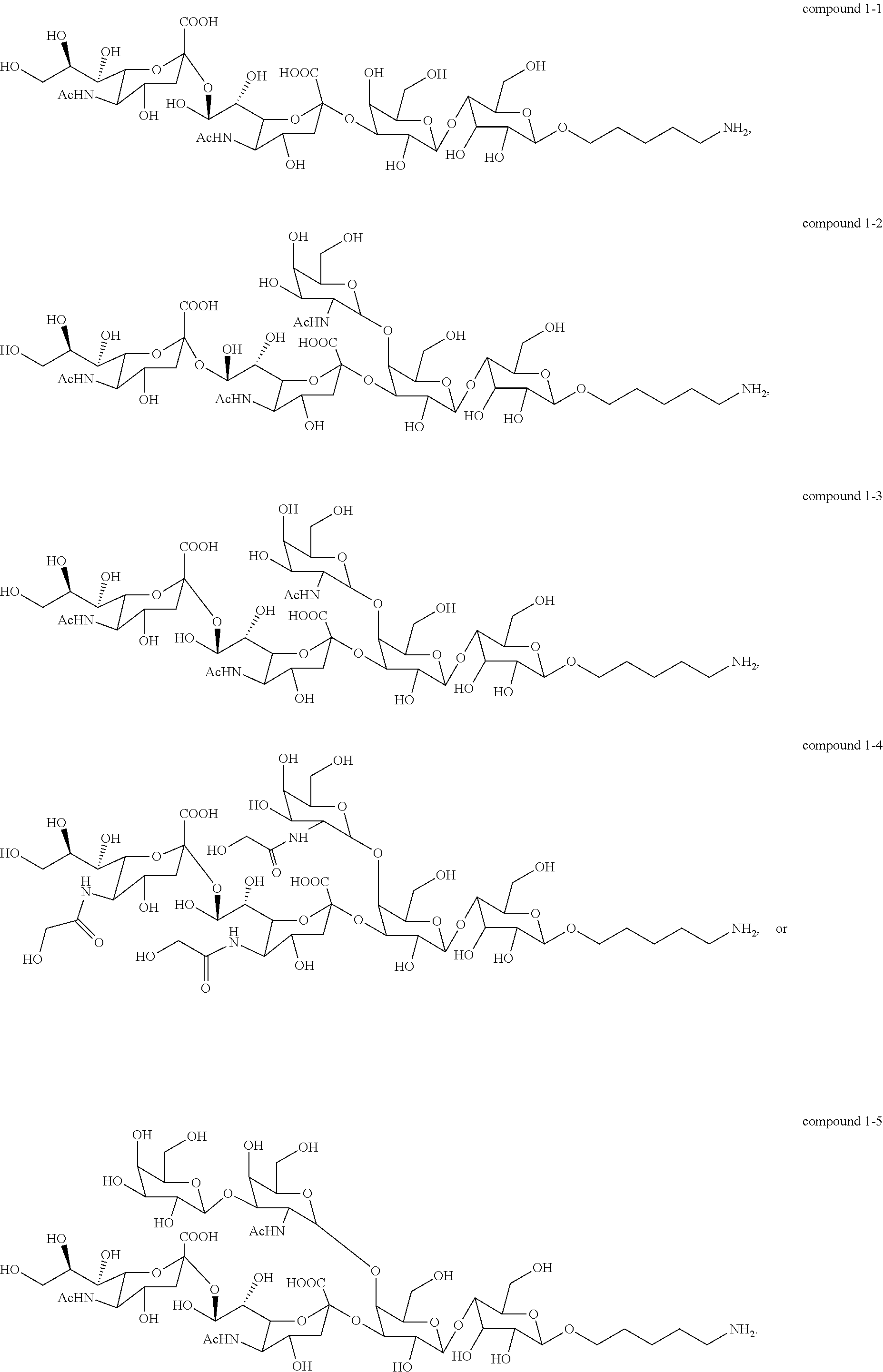

3. (canceled)

4. A pharmaceutical kit, comprising
a first compound having the structure of formula (I), 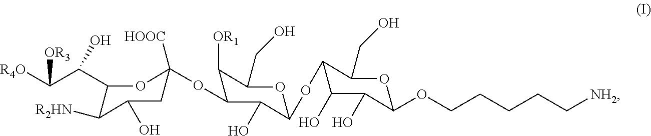
wherein
R₁ is H, or optionally substituted 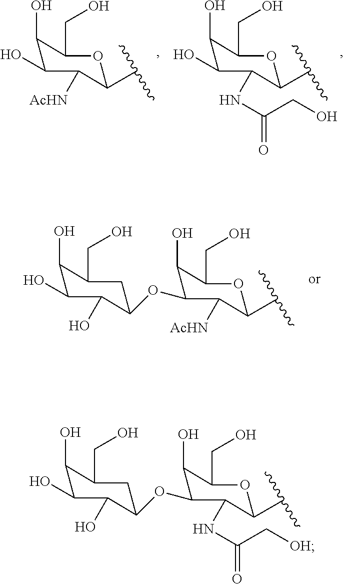 [SMILES: *C1OC(CO)C(O)C(O)C1NC(C)=O ; *C1OC(CO)C(O)C(O)C1NC(=O)CO ; *C1OC(CO)C(O)C(OC2CC(CO)C(O)C(O)C2O)C1NC(C)=O ; *C1OC(CO)C(O)C(OC2CC(CO)C(O)C(O)C2O)C1NC(=O)CO]
R₂ is optionally substituted acetyl or 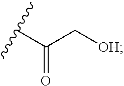 [SMILES: *C(=O)CO]
and
R₃ and R₄ are independently H, or optionally substituted 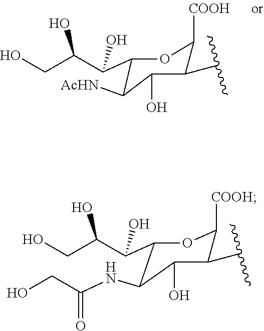 [SMILES: *C1C(C(=O)O)OC([C@H](O)[C@H](O)CO)C(NC(C)=O)C1O ; *C1C(C(=O)O)OC([C@H](O)[C@H](O)CO)C(NC(=O)CO)C1O]
and
a second compound selected from the group consisting of

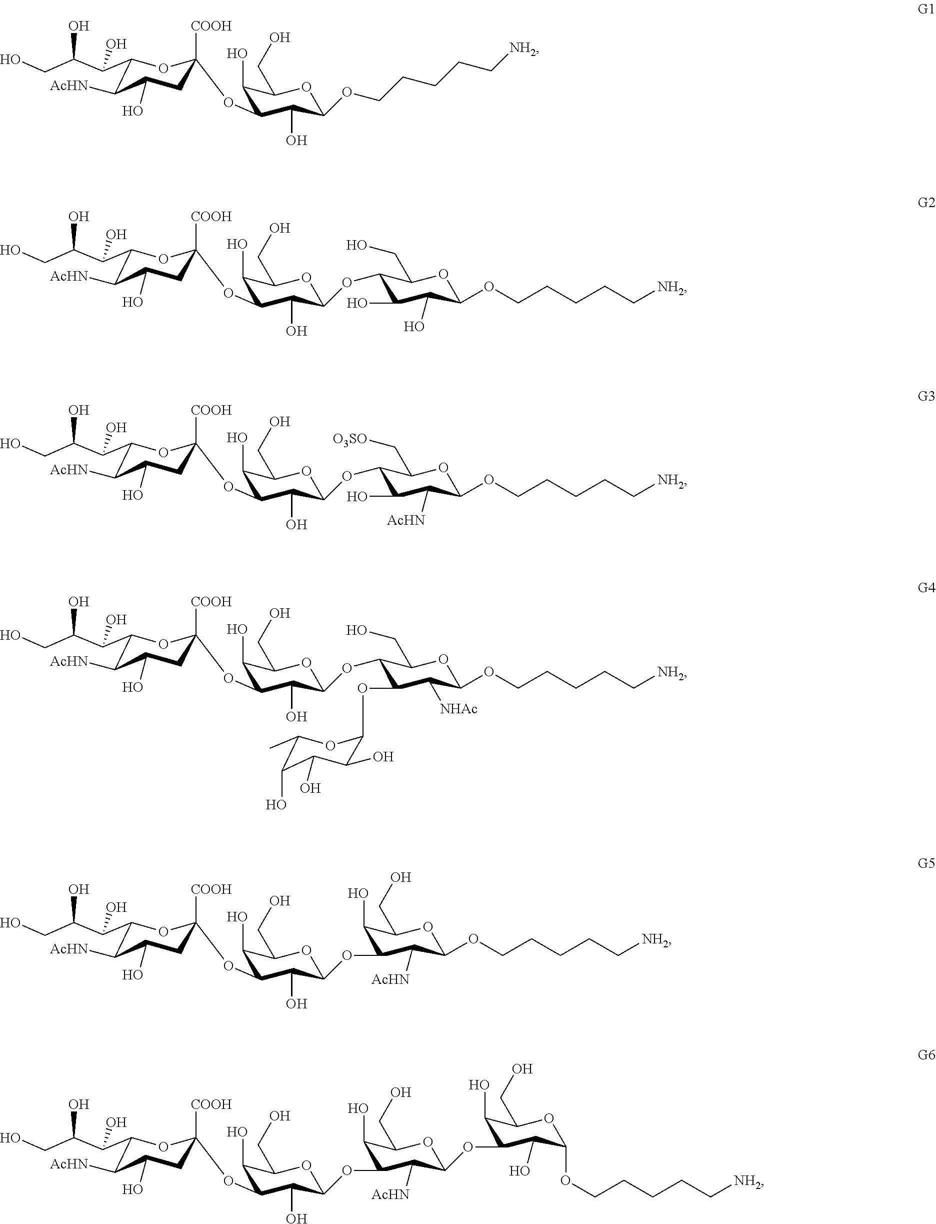

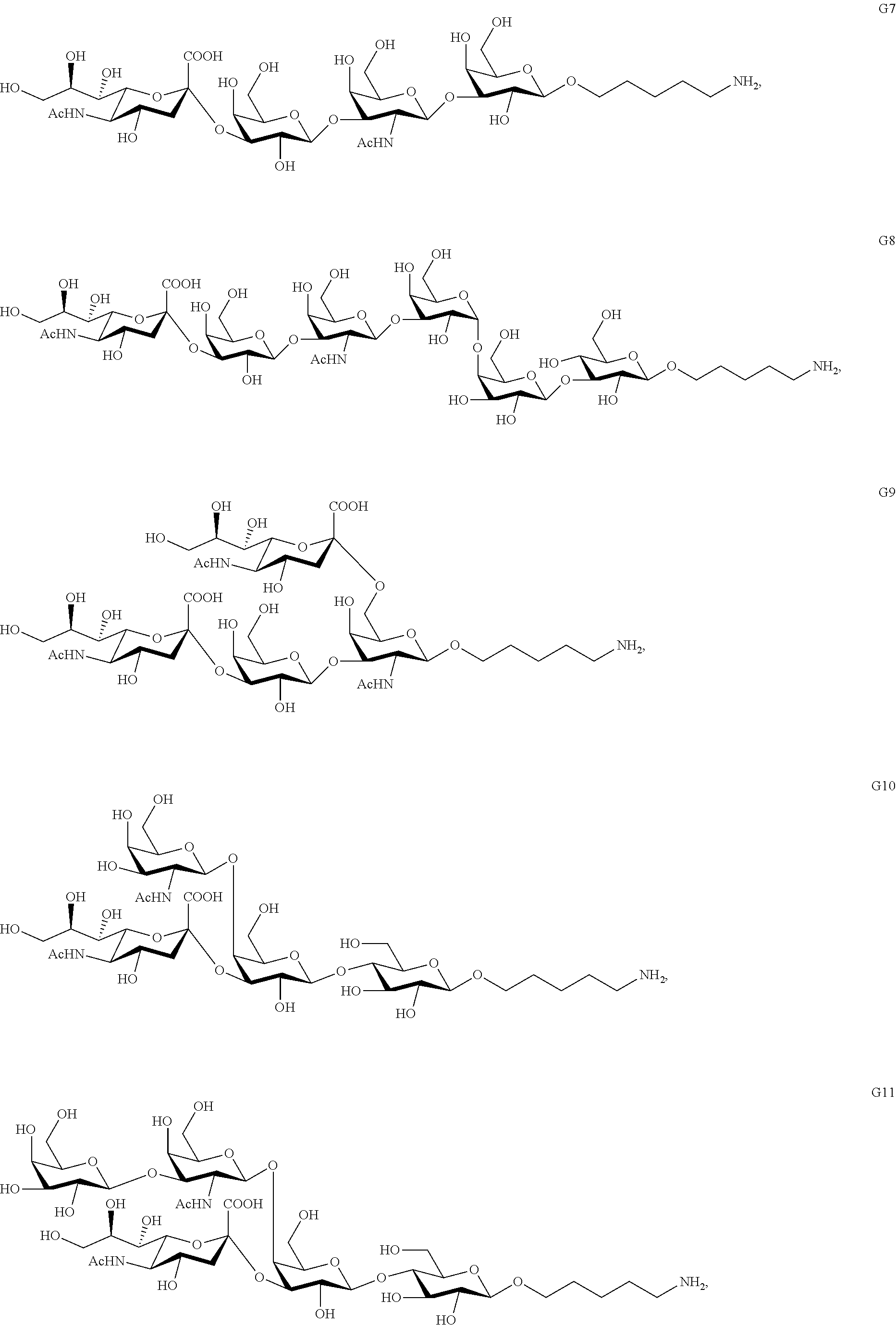

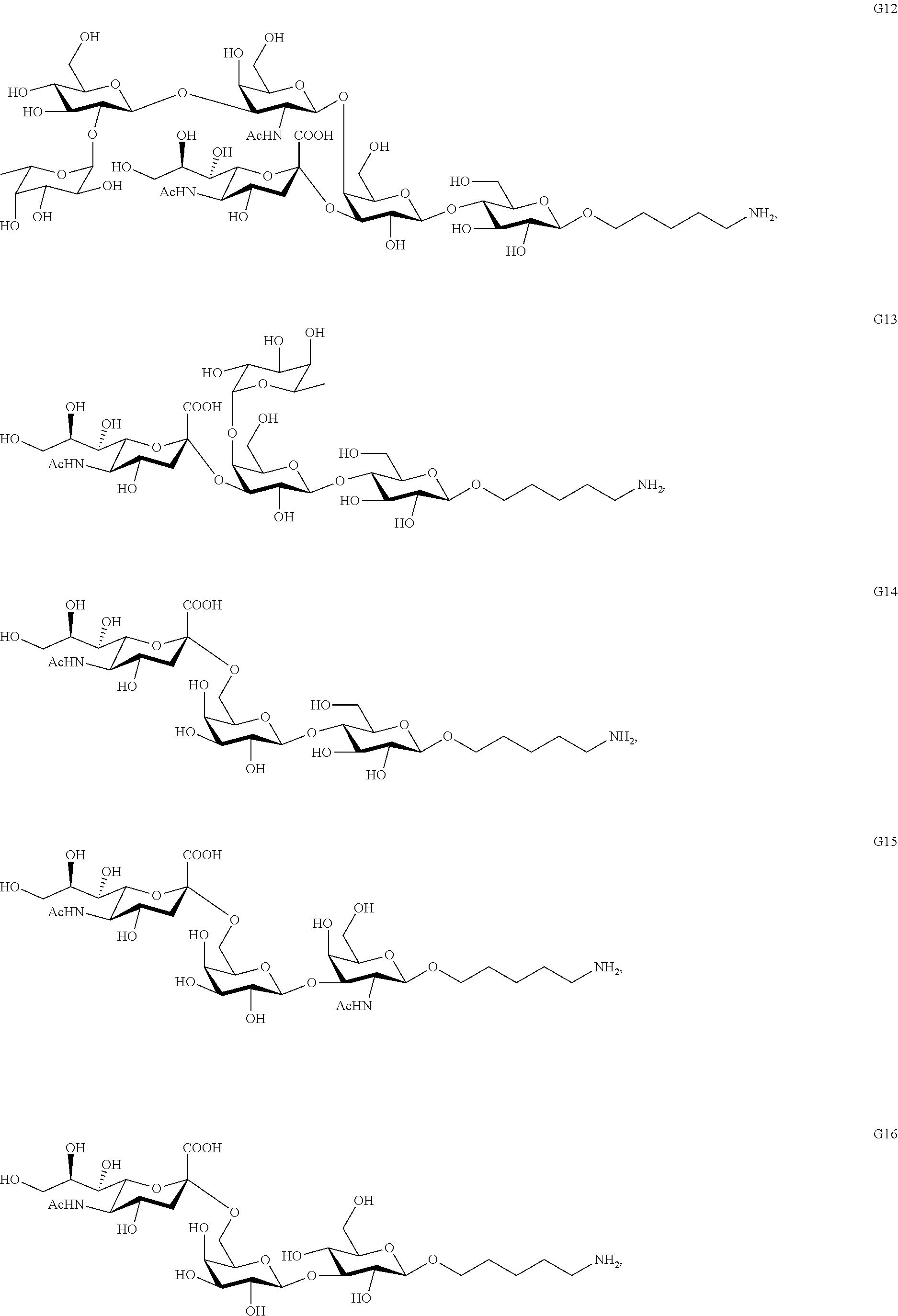

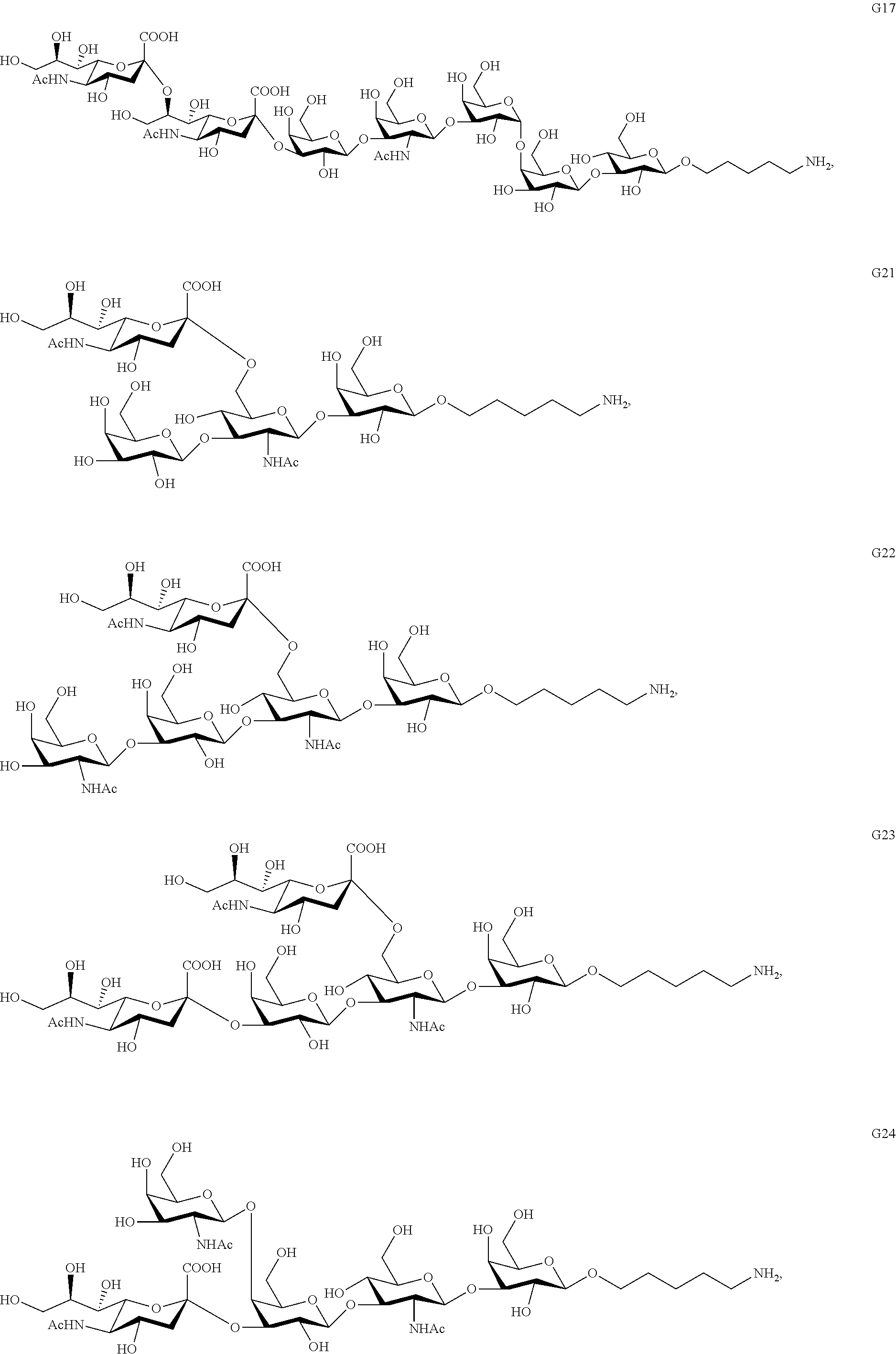

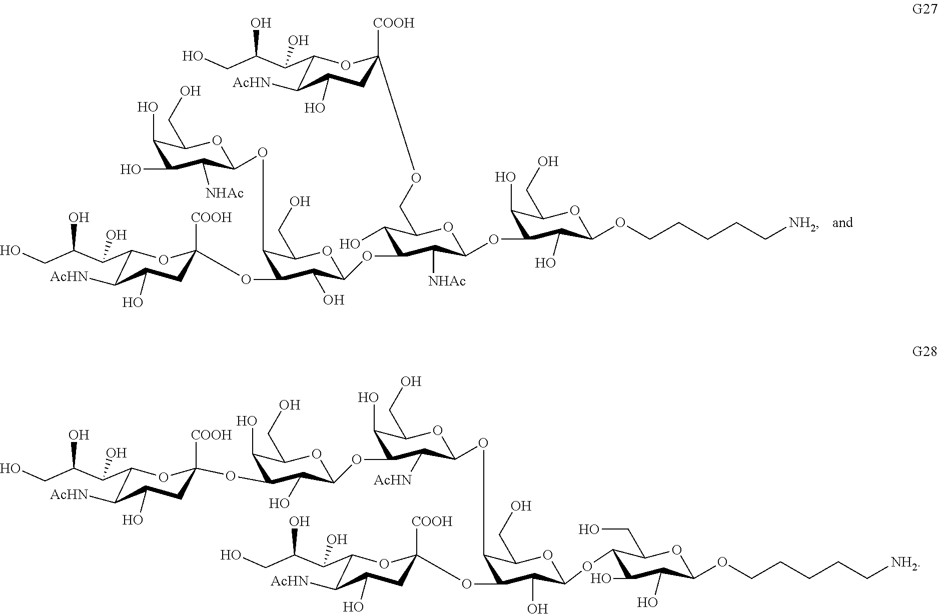

5. The pharmaceutical kit of claim 4, wherein the first compound is any of the followings,

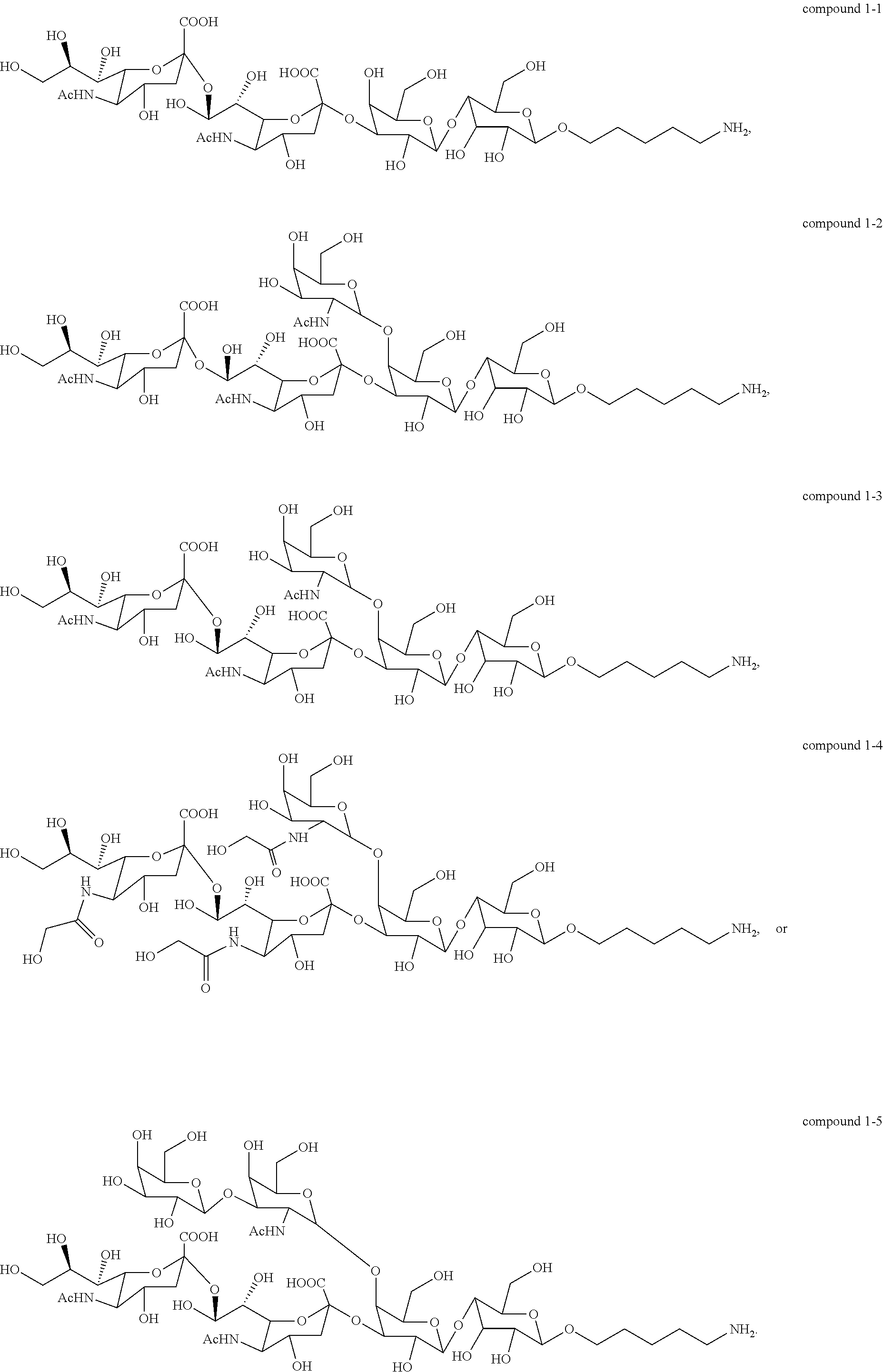

6. (canceled)

7. A method of making a prognosis or diagnosis of a neurodegenerative disease via use of a biological sample obtained from a subject, comprising,
(a) mixing the biological sample and the compound of claim 1 to form a first immunocomplex;
(b) reacting an anti-IgM antibody with the first immunocomplex of step (a) to give a second immunocomplex, wherein the anti-IgM antibody is conjugated with a reporter molecule;
(c) determining the signal level of the reporter of the step (b); and
(d) making the prognosis or diagnosis of the neurodegenerative disease based on the determination made in the step (c), wherein when the signal level is higher than that of a reference sample, then then subject has or is at risk of having the neurodegenerative disease.

8. The method of claim 7, wherein the reference sample is derived from a healthy subject.

9. The method of claim 7, wherein the biological sample is a whole blood sample, a serum sample, or a plasma sample.

10. The method of claim 7, wherein the reporter molecule is a tag molecule, a radioactive molecule, a fluorescent molecule, a phosphorescent molecule, a chemiluminescent molecule or an enzyme.

11. The method of claim 7, wherein the neurodegenerative disease is selected from the group consisting of Alzheimer's disease (AD), Parkinson disease (PD), Huntington's disease (HD), frontotemporal dementia (FTD), Friedreich's ataxia, age-related macular degeneration, and Creutzfeldt-Jakob disease.

12. The method of claim 7, wherein the subject is a human.

13. A method of treating a neurodegenerative disease in a subject, comprising,
(a) obtaining a biological sample from the subject;
(b) mixing the biological sample of step (a) and the compound of claim 1 to form a first immunocomplex;
(c) reacting an anti-IgM antibody with the first immunocomplex of step (b) to give a second immunocomplex, wherein the anti-IgM antibody is conjugated with a reporter molecule;
(d) determining the signal level of the reporter of the step (c); and
(e) administering to the subject an effective amount of an anti-neurodegenerative agent based on the determination made in the step (d), wherein the signal level in the biological sample of the subject is higher than that of a reference sample.

14. The method of claim 13, wherein the reference sample is derived from a healthy subject.

15. The method of claim 13, wherein the biological sample is a whole blood sample, a serum sample, or a plasma sample.

16. The method of claim 13, wherein the reporter molecule is a tag molecule, a radioactive molecule, a fluorescent molecule, a phosphorescent molecule, a chemiluminescent molecule or an enzyme.

17. The method of claim 13, wherein the neurodegenerative disease is selected from the group consisting of Alzheimer's disease (AD), Parkinson disease (PD), Huntington's disease (HD), frontotemporal dementia (FTD), Friedreich's ataxia, age-related macular degeneration, and Creutzfeldt-Jakob disease.

18. The method of claim 13, wherein the subject is a human.
